(19) United States
(12) Patent Application Publication
(10) Pub. No.: US 2005/0048070 A1
(43) Pub. Date: Mar. 3, 2005

(54) Cancer-associated epitope

(76) Inventors: Henrik Ditzel, San Diego, CA (US); Jens C. Jensenius, Odense M. (DK)

Correspondence Address: SCHWEGMAN, LUNDBERG, WOESSNER & KLUTH, P.A., P.O. BOX 2938, MINNEAPOLIS, MN, 55402, US

(21) Appl. No.: 10/883,020

(22) Filed: Jul. 1, 2004

Related U.S. Application Data
(63) Continuation of application No. PCT/US03/00297, filed on Jan. 3, 2003.
(60) Provisional application No. 60/345,208, filed on Jan. 3, 2002.

(51) Int. Cl. C12Q 1/68; A61K 39/00; C07H 21/04

(52) U.S. Cl. 424/185.1; 435/6; 435/69.1; 435/320.1; 435/325; 530/350; 536/23.5

(57) ABSTRACT

The present invention provides a cancer-associated epitope comprised of two polypeptides, where the first polypeptide is from cytokeratin K8 and the second polypeptide is from cytokeratin K18. The cancer-associated epitope becomes exposed during malignant transformation, particularly during malignant transformation of colon, breast, ovarian, renal, lung and testicular tissues. Exposure of the cancer-associated epitope is by cleavage and removal of N-terminal peptides from cytokeratins K8 and K18. The invention also provides binding entities, including antibodies, where the affinity of such binding entities for the cancer-associated epitope can be as high as about 10⁹ M⁻¹ in cancer tissues, more than 100-fold higher than for cytokeratin K8/K18 complexes in normal tissues. The invention provides cancer-associated epitopes, binding entities, antibodies and methods of using such epitopes, binding entities and antibodies for detection and treatment of cancer.

### RELATED APPLICATIONS

This application is a continuation under 35 U.S.C. 111 (a) of International Application No. PCT/US03/00297 filed Jan. 3, 2003 and published in English as WO 03/057168 A2 on Jul. 17, 2003, which claimed priority from U.S. Provisional Application Ser. No. 60/345,208 filed Jan. 3, 2002, which applications and publication are incorporated herein by reference.

### FIELD OF THE INVENTION

The present invention relates to cancer-associated epitopes, antibodies and polypeptide binding entities directed against such epitopes. The invention also relates to diagnostic agents comprising the epitopes, antibodies or binding entities, and to the use of the epitope, antibodies or binding entities for a variety of diagnostic or therapeutic purposes. Pharmaceutical compositions are also contemplated by the invention, where the compositions include the epitopes, antibodies or binding entities.

### BACKGROUND OF THE INVENTION

Malignant tumors sometimes express characteristic antigens or “markers” that offer a means for detecting and possibly treating the tumors. For example, antigens that are characteristic of the tumor may be purified and formulated and used to generate antibodies. The antibodies raised by these antigens can be used as detection tools to monitor the level of tumor marker in the host to track the course of the disease or the effectiveness of treatment. Antibodies have also been coupled to toxins and administered to treat cancer. In some instances, the antigens can be used as vaccines to stimulate an antibody response and a cellular immune response within a cancer patient and thereby discourage the growth and spread of the cancer.

Glandular epithelia cells contain a network of intermediate filaments that predominantly consists of complexes of cytokeratin 8 (K8) and cytokeratin 18 (K18). These filaments provide resilience in response to mechanical stress by forming a stable network attached to specific cell-cell contacts of the desmosome type (1). Intermediate filaments can be classified into groups, which in higher eukaryotes are expressed in a tissue-specific and cell type-restricted pattern (2). In epithelia cells, intermediate filaments consist of stoichiometrically equal amounts of type I (smaller, acidic) and type II (larger, neutral or basic) cytokeratin polypeptides that form strongly interacting heterodimers (3-5).

Each cytokeratin polypeptide consists of a central 300-350 amino acid α-helical rod domain that is flanked by non-helical head (N-terminal) and tail (C-terminal) domains of various lengths and compositions. The rod domain can be further subdivided into four sub-domains (1A, 1B, 2A and 2B), which are interspaced by short non-helical linkers (L1, L12, L2). Likewise, the head domain can be subdivided into three domains: the end domain (E1), the variable domain (V1) and a region of sequence homology (H1) nearest to the rod domain (6).

The assembly of intermediate filaments appears to involve several association steps that depend on interactions between different domains. In general, a type I and a type II cytokeratin polypeptide align in parallel to yield a coiled coil heterodimer (7,8). Subsequently, a tetramer is formed by anti-parallel, staggered, side-by-side aggregation of two dimers. The tetramers polymerize end to end to form a protofilament, and eight protofilaments then combine to produce the final 10 nm filament (9).

The assembled rods form a protofilament backbone structure from two coiled coil subunits. However, the head and the tail domain are not thought to be part of the filamentous backbone. Instead, the head and tail domain appear to protrude laterally and to contribute to protofilament and intermediate filaments packing. The head and tail domains may also contribute to intermediate filament interaction with other cellular components (10-12). Thus, cytokeratins lacking the head and tail domains are generally capable of coiled coil and higher order lateral interactions, but are deficient in filament elongation (13).

Cytokeratin 8 (K8) type I and cytokeratin 18 (K18) type II are the major components of intermediate filaments of simple or single layer epithelia, such as those of the intestine, liver and breast ducts (4). These two cytokeratins form heterodimers and filaments. Deletion studies of K8 and K18 cytokeratins have shown that the head domains play a crucial role in forming heterodimers and filaments. Co-transfection of head-deleted K8 and head-deleted K18 resulted in the formation of a dispersed non-fibrillar pattern, while co-transfection of a combination of one headless plus one intact cytokeratin resulted in the formation of cytoplasmic granules or fibrils (12). More detailed analysis showed that only short and irregular intermediate filaments were generated when K8 and K18 were N-terminally truncated by deleting the first 66 amino acid of each of the cytokeratins (13). The whole, or nearly the whole, H1 region of the head domain was required for generation of these short filaments. Only tetramers were generated when a major part of the H1 domain was additionally removed to form a complex between a truncated K8 (amino acids 75-483) and a truncated K18 (amino acids 67-385) (13). The importance of H1 region apparently relates to its involvement in the alignment of the two heterodimers and to the stabilization of the formed heterotetramer complexes.

The precise function of K8 and K18 remains largely unclear, although recent data indicates that both cytokeratins are important in natural development. A mutant of K18 (arg89→cys), expressed as a dominant trait in transgenic mice, resulted in marked disruption of the liver and pancreas intermediate filament network, leading to hepatocyte instability and associated liver inflammation and necrosis (14,15). The phenotype of K8 or K19 knockout mice included complete or partial midgestational embryonic lethality depending on the genetic background, female sterility and adult colorectal hyperplasia in the surviving animals (16-18). Other data have suggested that K8/K18 filaments play a role in multiple drug resistance (19-21).

During cell transformation and tumor development, the cell type specificities of K8 and K18 are conserved, making them useful as clinical histopathological markers for tissue type identification (22-24). Given that the cell type specificities of K8 and K18 are conserved during cell transformation and tumor development, one would not expect that the K8 and K18 cytokeratins would exhibit a new antigenic epitope in cancerous cells.

### SUMMARY OF THE INVENTION

The invention provides an isolated cancer-associated epitope comprising two separate polypeptides. The first polypeptide can have SEQ ID NO:3 of cytokeratin 8 and the second polypeptide can have SEQ ID NO:4 of cytokeratin 18. Alternatively, the first polypeptide can have SEQ ID NO:5 of cytokeratin 8 and the second polypeptide can have SEQ ID NO:6 of cytokeratin 18. Moreover, the first polypeptide can have SEQ ID NO:3 of cytokeratin 8 and the second polypeptide can have SEQ ID NO:6 of cytokeratin 18. The first polypeptide can also have SEQ ID NO:5 of cytokeratin 8 and the second polypeptide can have SEQ ID NO:4 of cytokeratin 18.

Such isolated epitopes can be detected in filamentous cytoplasmic structures of adenocarcinoma cells but are not substantially detected in normal cells. Examples of adenocarcinomas where these epitopes can be detected include colon adenocarcinoma, ovarian adenocarcinoma, renal adenocarcinoma, mammary adenocarcinoma, lung adenocarcinoma, pancreatic adenocarcinoma and non-seminomal testis carcinoma cells. These epitopes are useful for making cancer-specific antibodies, and for diagnosing cancer by detecting either the antigenic epitopes or antibodies directed against these epitopes in the blood, serum, feces or urine of a cancer patient. Accordingly, in one embodiment, the epitopes are provided in a kit.

In another embodiment, the invention provides an antibody or other binding entity that can bind any of the cancer-associated epitopes of the invention. In one embodiment, the antibody or binding entity can include a polypeptide comprising any one of SEQ ID NO:7-35. Preferred antibodies or binding entities include polypeptides comprising any one of SEQ ID NO:21-35, or a combination thereof. In another embodiment, the invention is directed to a polypeptide comprising any combination of SEQ ID NO:7-33, wherein the polypeptide that can bind an epitope of the invention. The antibody or binding entity can be encoded by a nucleic acid comprising any one of SEQ ID NO:36-39. Such a binding entity or antibody can detect the cancer-associated epitope in filamentous cytoplasmic structures of colon adenocarcinoma, ovarian adenocarcinoma, renal adenocarcinoma, mammary adenocarcinoma, lung adenocarcinoma, pancreatic adenocarcinoma and non-seminomal testis carcinoma cells. The antibody or binding entity can have a label or diagnostic imaging agent attached to it. The invention also provides such compositions and kits containing the binding entities or antibodies. Preferably, when antibodies are employed, the antibody is not a COU-1 monoclonal antibody.

The invention further provides a method of detecting adenocarcinoma by contacting an antibody or binding entity of the invention with a test sample and detecting whether the antibody or binding entity binds to a cancer-associated epitope. The antibodies and binding entities can have a label or diagnostic imaging agent attached thereto.

The invention also provides a method of treating cancer in a mammal by administering a therapeutically effective amount of an antibody or binding entity of the invention that can bind to the cancer-associated epitope. Such an antibody or binding entity can have a therapeutically useful agent attached thereto.

The invention further provides a method of treating cancer in a mammal comprising administering a therapeutically effective amount of a cancer-associated epitope of the invention.

The invention also provides a method of treating cancer in a mammal comprising administering a therapeutically effective amount of a protease inhibitor that can inhibit formation of the cancer-associated epitopes of the invention by inhibiting the protease(s) that cleave cytokeratin 8 and cytokeratin 18. In one embodiment, the protease is a trypsin-like protease and the inhibitor is a serine protease inhibitor or a trypsin inhibitor.

The invention also provides a method of identifying a mutant antibody comprising fusing a nucleic acid encoding a polypeptide having any one of SEQ ID NO:7-35 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to a cancer-associated epitope of the invention.

### DESCRIPTION OF THE FIGURES

FIG. 1 illustrates the purification of cytokeratin from colon cancer tissue.

FIG. 1A provides an elution profile (OD₂₈₀, solid line) of cytokeratin-enriched material applied to a QFF anion-exchange column (100-ml bed volume) in SDS-containing buffer and eluted with a linear gradient to 1 M NaCl (dashed line). Fractions containing reactivity with COU-1 were observed in the first and second peaks of the salt gradient.

FIG. 1B provides an expanded view of a region the elution profile shown in FIG. 1A. This expanded profile provides a comparison of OD₂₈₀ absorption (solid line), salt gradient (dashed line) and COU-1 reactivity (dotted line) for fractions (30-53). Proteins from these fractions were coated onto ELISA wells and reacted with the COU-1 antibody.

FIG. 1C provides a Coomassie-stained blot of electrophoretically separated proteins from fractions 41-50, raw homogenate (H), and material applied to the QFF anion-exchange column (S). An extract of the colon cancer cell line Colon 137 (C137) was included as control.

FIG. 1D provides a Western blot (stained with COU-1) of electrophoretically separated proteins from fractions 41-50, raw homogenate (H), and material applied to the QFF anion-exchange column (S). An extract of the colon cancer cell line Colon 137 (C137) was included as control. This blot illustrates the purity of the cytokeratins obtained from the QFF column and the reactivity of the COU-1 antibodies with 3 bands at molecular weights of 42-46 kDa.

FIG. 2 provides a Western blot analysis of cytokeratin preparations purified from colon cancer tissue or from normal colon epithelia under identical conditions. Homogenate (homog) and anion-exchange chromatography-purified material (QFF) was applied to the SDS-PAGE gel at 10-fold dilutions (1:1, 1:10, 1:100). The proteins were transferred to PVDF membranes and stained with COU-1 or murine anti-K18 Mab. While the anti-K18 antibody intensely stained cytokeratin preparations from normal and malignant colon epithelia, COU-1 intensely stained only cytokeratin proteins from cancer tissue (three bands of about 42-46 kDa), and not cytokeratin proteins from normal epithelia.

FIG. 3 illustrates the SDS-PAGE separation and N-terminal sequencing of cytokeratins purified from colon cancer tissue and further illustrates the presence of N-terminally-truncated K8, K18 and K19 cytokeratins in these colon cancer tissues.

FIG. 3A is a PVDF membrane blot of SDS-PAGE-separated purified cytokeratin. Region (a) of this membrane was stained with Coomassie. Strips of the membrane were also stained with the COU-1 antibody (region b), a mouse anti-K8 antibody (region c) and a mouse anti-K18 antibody (region d). These data illustrate that ten different protein bands (1-10) can be detected. These ten bands were each N-terminally sequenced.

FIG. 3B provides the amino acid sequences of cytokeratin proteins isolated from colon cancer tissues as determined by N-terminal sequencing (SEQ ID NOs:54-61). In addition, the reactivity of the different isolated cytokeratin proteins with a panel of K8/K18 antibodies is shown.

FIG. 4 provides a map of the structural domains of the K8 and K18 cytokeratins (center). The secondary structural domains of the cytokeratin polypeptides were identified from the amino acid sequences. A central rod domain is shown that is flanked by a non-helical N-terminal head domain and a non-helical C-terminal tail domain. Domains 1A, 1B, 2A and 2B are α-helical subdomains of the rod interspaced by linkers L1, L12 and L2. This figure also provides a schematic representation of the K8 and K18 N-terminal and C-terminal deletion proteins. Deletion protein names provide the starting and ending amino acid residue numbers of the deletion protein. All deletion proteins were expressed as GST fusion proteins. The positive (+) and negative (−) reactivity of the deletion protein fragments with COU-1 after incubation with the complementary keratin is shown in parentheses.

FIG. 5 provides a Western blot analysis of a panel of C-terminal deleted K18 fragments. SDS-PAGE gels containing a panel of C-terminal deleted K18 fragments expressed as GST fusion proteins were run in parallel, transferred to PVDF membranes and stained with either a goat anti-GST antibody (A), COU-1 (B) or a mouse anti-K18 antibody (CY-90) (D).

FIG. 5A is a Western blot of an electrophoretically separated panel of C-terminal deleted K18 protein fragments expressed as GST fusion proteins that was stained with a goat anti-GST antibody. The identity of the different K18 protein fragments is provided at the top, where the numbers indicate which amino acids are present in the different K18 protein fragments. A GST protein preparation was used a positive control for GST antibody staining. An MCF7 cancer cell lysate was used as positive control for the cytokeratin epitope (no staining is visible because the GST protein is not present in the lysate). The GST antibody staining demonstrated that all K18 fragments were expressed well and at approximately the same levels.

FIG. 5B is a replica of the Western blot of an electrophoretically separated panel of C-terminally deleted K18 fragments provided in FIG. 5A that was stained with the COU-1 antibody. On this blot, COU-1 only reacted with MCF7 cancer cell lysate used as positive control. No substantial binding of COU-1 to individual K18 fragments was observed.

FIG. 5C is a replica of the Western blot of an electrophoretically separated panel of C-terminally deleted K18 fragments provided in FIG. 5A. However, this blot was incubated with purified intact K8 prior to staining COU-1. COU-1 bound strongly to some of the K18 fragments when complexed with K8.

FIG. 5D is a replica of the Western blot of an electrophoretically separated panel of C-terminally deleted K18 fragments provided in FIG. 5A that was stained with a mouse anti-K18 antibody (CY-90). The CY-90 antibody reacted with an epitope corresponding to residues 340-390 in the C-terminal part of non-complexed K18.

FIG. 6 provides an SDS-PAGE and Western blot analysis of a panel of C-terminal deleted K8 fragments. SDS-PAGE gels containing a panel of C-terminal deleted K8 fragments expressed as GST fusion proteins were run in parallel and stained with Coomassie Blue (A), or transferred to PVDF membranes and stained with COU-1 (B) or incubated with purified intact K18 prior to staining HMab COU-1 (C).

FIG. 6A is an SDS-PAGE gel of an electrophoretically separated panel of C-terminal deleted K8 fragments stained with Coomassie Blue. The identity of the different K8 protein fragments is provided at the top, where the numbers indicate the range of amino acids present in the different K18 protein fragments. Coomassie Blue staining demonstrated that all fragments were expressed approximately equally well.

FIG. 6B is a replica of the Western blot of an electrophoretically-separated panel of C-terminally deleted K8 fragments provided in FIG. 6A that was stained with the COU-1 antibody. No binding of COU-1 to individual K8 fragments was observed. On this blot, COU-1 only reacted with the positive control, a MCF7 cancer cell lysate.

FIG. 6C is a replica of the Western blot of an electrophoretically-separated panel of C-terminally deleted K8 fragments provided in FIG. 6A that was incubated with purified intact K18 prior to staining HMab COU-1. COU-1 bound strongly to some of the K8 fragments because when they had formed complexes with K18.

FIG. 7 provides a Western blot analysis of a panel of C-terminally deleted K8 or K18 fragments that were electrophoretically separated, transferred to a PVDF membrane and then incubated with different purified C-terminally deleted K8 or K18 fragments to form K8/K18 complexes prior to staining with COU-1.

FIG. 7A provides a Western blot analysis of a panel of C-terminally deleted K18(1-72), K18(1-124), K18(1-187) and intact K18 protein fragments that were electrophoretically separated and transferred to a PVDF membrane. The membrane was then incubated with purified C-terminally deleted K8(1-129) fragment and stained with COU-1. The COU-1 antibodies bound strongly to K18(1-124)/K8(1-129) complexes.

FIG. 7B is a replica of the Western blot of an electrophoretically-separated panel of C-terminally deleted K18 fragments provided in FIG. 7A that was incubated with purified C-terminally deleted K8(1-233) fragment and stained with COU-1. COU-1 staining was absent or only minimally observed for the K18(1-187)/K8(1-233) complex.

FIG. 7C is a replica of the Western blot of an electrophoretically-separated panel of C-terminally deleted K18 fragments provided in FIG. 7A that was incubated with purified intact K8 and stained with COU-1. COU-1 staining was absent or only minimally observed for the K18(1-187)/intact K8 complex.

FIG. 7D provides a Western blot analysis of a panel of C-terminally deleted K8(1-85), K8(1-129), K8(1-233) and intact K8 polypeptides that were electrophoretically separated and transferred to a PVDF membrane. The membrane was then incubated with purified K18(1-124) and stained with COU-1. The COU-1 antibody recognized K8(1-129) complexed with K18(1-124), and K8(1-233) complexed with K18(1-124). No COU-1 binding was observed with complexes containing K8(1-85).

FIG. 7E is a replica of the Western blot of an electrophoretically-separated panel of C-terminally deleted K8 fragments provided in FIG. 7D that was incubated with purified K18(1-187) and stained with COU-1. The COU-1 antibody recognized K8(1-129) complexed with K18(1-187) and K8(1-233) complexed with K18(1-187). No COU-1 binding was observed with any complexes containing K8(1-85).

FIG. 7F is a replica of the Western blot of an electrophoretically-separated panel of C-terminally deleted K8 fragments provided in FIG. 7D that was incubated with purified K18(1-213) and stained with COU-1. The COU-1 antibody recognized K18(1-213) complexed with K8(1-129) and K18(1-213) complexed with K8(1-233) K18(1-187). No COU-1 binding was observed with any complexes containing K8(1-85).

FIG. 8 provides a schematic representation of the N-terminal head domain and the adjacent rod domain of K8/K18 heterotypic complex.

FIG. 8A identifies the sites where K8 (SEQ ID NO:62) and K18 (SEQ ID NO:63) are proteolytically cleaved (arrows). For cytokeratin K8, cleavage was after Arg-22, after Arg-39, after Val-65 and after Leu-75. For cytokeratin K18, cleavage was after Arg-49 and after Ile-67. The positions of residues that are post-translationally phosphorylated (PO₄, P) or glycosylated (GlcNac, G) are also identified.

FIG. 8B is a schematic diagram illustrating how cleavage of the N-terminal head domain of K8 and K18 cytokeratins can cause a conformational change allowing the COU-1 antibody to access the epitope. This diagram is consistent with observations made in vivo in cancer cells and made on the formation of recombinant K8/K18 complexes. This diagram further illustrates how in vitro deletion of a major portion of the C-terminal domain of one of the two cytokeratin proteins may also artificially expose the COU-1 epitope. This diagram is also consistent with complex formation studies on a panel of recombinant K8/K18 deletion proteins.

FIG. 9 illustrates that COU-1 binds preferentially to heterotypic complexes containing N-terminally deleted K8 fragments. This figure provides a PVDF membrane blot of SDS-PAGE-separated K8 and K18 proteins. Region A has intact K8 or K8 (66-483) proteins that were electrophoretically-separated and then reacted with purified K18 (50-430) prior to staining with COU-1. Region B has intact K8 or K8 (66-483) proteins incubated with purified intact K18 prior to staining with COU-1. Region C has K18 (50-430) and intact K18 that were electrophoretically-separated and then incubated with purified K8 (66-483) prior to staining with COU-1. Strong staining of a band of about 75 kDa (K8/K18 proteins+the GST fusion protein) was observed in lanes containing K8(66-483)/K18(50-430) and K8(66-483)/intact K18 with COU-1. Region D has K18(50-430) and intact K18 proteins that were electrophoretically-separated and then incubated with purified intact K8 prior to staining with COU-1. Only weak staining was observed to intact K8/K18 (50-430) and intact K8/intact K18.

FIG. 10 illustrates that COU-1 binds preferentially to truncated forms of recombinant heterotypic K8/K18 complexes as measured by ELISA. Heterotypic complexes were generated by combining purified recombinant K8(1-129) or intact K8 with purified recombinant K18(1-124) or intact K18 in equal molar ratio. A 5 μg/ml solution of these complexes was used to coat ELISA plates. The ELISA plates were incubated with COU-1 in serial dilutions. Bound COU-1 was detected and visualized with an AP-labeled secondary anti-human kappa antibody and para-nitrophenylphosphate. As shown, the K8(1-129)/intact K18 complex (diamond symbols) and the K8/K18(1-124) (circles) bound more COU-1 antibodies than the intact K8/intact K18 complex.

FIG. 11 illustrates the distribution of N-terminally-truncated K8/K18 complexes (A and E) and K18 (B and F) in breast cancer cells. Ethanol-fixed MCF7 breast cancer cells were separately incubated with COU-1 antibodies (A and E) and CY90 monoclonal antibodies (B and F), which binds to intact K18. Bound COU-1 was detected with FITC-goat anti-human y-chain antibody (green) and bound CY90 detected with a Texas Red-goat-anti-mouse IgG antibody (red). DIC images (D and H) were included to visualize the composition of the cells. Partial co-localization, as visualized by yellow in the merged images (C and G), was observed between the two antibodies. However, N-terminally truncated K8/K18 complexes were predominantly found in newly-formed, proliferating cancer cells (arrows), whereas K18 structures were equally present in all cells (arrowheads).

FIG. 12 shows the cellular distribution of the N-terminally-truncated K8/K18 complex recognized by COU-1 (A and E) and K18 recognized by Mab CY-90 (B and F) in breast cancer cells. MCF7 breast cancer cells were processed and stained as described in FIG. 11. DIC images (D and H) were included to visualize the composition of the cells. While whole intermediate filaments were stained with Mab CY-90 (arrowheads), COU-1 (arrows) only stained short fibrils and globular structures. Some co-localization of the two antibodies, as visualized by yellow in the merged images (C and G), was observed.

FIG. 13 provides the amino acid sequences for the variable heavy and light chain of the human monoclonal antibody COU-1 in comparison to closest known germ-line sequences (SEQ ID NOs:7-20).

### DETAILED DESCRIPTION OF THE INVENTION

According to the invention, the separate K8 cytokeratin polypeptide joins with the K18 cytokeratin polypeptide to form an antigenic epitope that is only visible in cancerous, and not in normal, cells. Such neoepitopes are generated by specific proteolytic cleavage of K8/K18 complexes in carcinoma cells. The new epitopes visible in cancer cells are used to generate antibodies or binding entities that are diagnostic of cancer and that are useful for treatment of cancer patients.

Definitions

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv) so long as they exhibit the desired biological activity.

A “binding entity,” as used herein, is a polypeptide that can bind to the epitope identified by the invention. For example, a binding entity of the invention is a polypeptide that can bind to an epitope comprising two separate polypeptides, a cytokeratin 8 polypeptide and a cytokeratin 18 polypeptide, wherein the cytokeratin 8 polypeptide comprises SEQ ID NO:3 or SEQ ID NO:5, and the cytokeratin 18 polypeptide comprises SEQ ID NO:4 or SEQ ID NO:6.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include in colon adenocarcinoma, ovarian adenocarcinoma, renal adenocarcinoma, mammary adenocarcinoma, lung adenocarcinoma, pancreatic adenocarcinoma and non-seminomal testis carcinoma tissues.

The COU-1 antibody is a monoclonal antibody produced by the human hybridoma cell line B9165 (ECACC 87040201). It can bind to a carcinoma-associated antigen that has an apparent molecular weight of about 43,000 and an isoelectric point in the range of about 5.4-6.2.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The “derivative” of a reference antigenic epitope, antibody, nucleic acid, protein, polypeptide or peptide, has related but different sequence or chemical structure than the respective reference antigenic epitope, antibody, nucleic acid, protein, polypeptide or peptide. Such a derivative antigenic epitope, antibody, nucleic acid, protein, polypeptide or peptide is generally made purposefully to enhance or incorporate some chemical, physical or functional property that is absent or only weakly present in the reference antigenic epitope, antibody, nucleic acid, protein, polypeptide or peptide. A derivative nucleic acid differs in nucleotide sequence from a reference nucleic acid whereas a derivative antigenic epitope, antibody, protein, polypeptide or peptide differs in amino acid sequence from the reference antigenic epitope, antibody, protein, polypeptide or peptide, respectively. Such sequence differences include one or more substitutions, insertions, additions, deletions, fusions and truncations, which can be present in any combination. Differences can be minor (e.g., a difference of one nucleotide or amino acid), or more substantial, involving several or many nucleotides or amino acids. However, the sequence of the derivative is not so different from the reference that one of skill in the art would not recognize that the derivative and reference are related in structure and/or function. Generally, differences are limited so that the reference and the derivative are closely similar overall and, in many regions, identical. A “variant” differs from a “derivative” nucleic acid, protein, polypeptide or peptide in that the variant can have silent structural differences that do not significantly change the chemical, physical or functional properties of the reference nucleic acid, protein, polypeptide or peptide. In contrast, the differences between the reference and derivative nucleic acid, protein, polypeptide or peptide are intentional changes made to improve one or more chemical, physical or functional properties of the reference nucleic acid, protein, polypeptide or peptide.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

A “liposome” is a small vesicle composed of various types of lipids, phospholipids and/or surfactant that is useful for delivery of a drug (such as the antigenic epitopes and antibody mutants disclosed herein and, optionally, a chemotherapeutic agent) to a mammal. The components of the liposome are commonly arranged in a bilayer formation, similar to the lipid arrangement of biological membranes.

“Mammal” refers to any animal classified as a mammal, including human, domestic and farm animals, nonhuman primates, and zoo, sports, or pet animals, such as dogs, horses, cats, cows, etc.

Nucleic acid is “operably linked” when it is placed into a functional relationship with another nucleic acid sequence. This can be a gene and a regulatory sequence(s) that are connected in such a way as to permit gene expression when the appropriate molecules (e.g., transcriptional activator proteins) are bound to the regulatory sequences(s). For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucteotide adaptors or linkers are used in accordance with conventional practice.

The terms “protein,” 37 polypeptide” and “peptide” are used interchangeably. They refer to a chain of two (2) or more amino acids that are linked together with peptide or amide bonds, regardless of post-translational modification (e.g., glycosylation or phosphorylation). Antigens, epitopes and antibodies are specifically intended to be within the scope of this definition. The polypeptides of this invention may comprise more than one subunit, where each subunit is encoded by a separate DNA sequence.

The phrase “substantially identical” with respect to an antigen, antibody or binding entity polypeptide sequence shall be construed as a polypeptide exhibiting at least 70%, preferably 75%, more preferably 80%, more preferably 85%, even more preferably 90%, even more preferably 95% and especially preferably 97% or 98% sequence identity to the reference polypeptide sequence. For polypeptides, the length of the comparison sequences will generally be at least 25 amino acids. For nucleic acids, the length will generally be at least 75 nucleotides.

The “variant” of a reference antigenic epitope, antibody segment, binding entity, nucleic acid, protein, polypeptide or peptide, is an antigenic epitope, antibody segment, binding entity, nucleic acid, protein, polypeptide or peptide, respectively, with a related but different sequence than the respective reference antigenic epitope, antibody segment, binding entity, nucleic acid, protein, polypeptide or peptide. The differences between variant and reference antigenic epitopes, antibody segments, binding entities, nucleic acids, proteins, polypeptides or peptides are silent or conservative differences. A variant nucleic acid differs in nucleotide sequence from a reference nucleic acid whereas a variant antigenic epitope, antibody segment, binding entity, protein, polypeptide or peptide differs in amino acid sequence from the reference antigenic epitope, antibody segment, binding entity, protein, polypeptide or peptide, respectively. A variant and reference antigenic epitope, antibody segment, binding entity, nucleic acid, protein, polypeptide or peptide may differ in sequence by one or more substitutions, insertions, additions, deletions, fusions and truncations, which may be present in any combination. Differences can be minor (e.g., a difference of one nucleotide or amino acid), or more substantial. However, the structure and function of the variant is not so different from the reference that one of skill in the art would not recognize that the variant and reference are related in structure and/or function. Generally, differences are limited so that the reference and the variant are closely similar overall and, in many regions, identical.

Epitope

According to the invention, one or more novel neoepitopes that are immunologically recognizable are generated in a variety of adenocarcinoma cells through specific proteolytic cleavage of cytokeratin K8 and cytokeratin K18 proteins. Normal, non-cancerous cells do not display such neoepitopes. The cytokeratin K8 and cytokeratin K18 proteins are separate proteins. However, they do form a cytokeratin K8/cytokeratin K18 complex. The immunologically recognizable neoepitope contains amino acids from both the cytokeratin K8 and cytokeratin K18 proteins.

The epitope of the invention is not substantially present in normal tissues. However, the epitope becomes exposed in colon adenocarcinoma, ovarian adenocarcinoma, renal adenocarcinoma, mammary adenocarcinoma, lung adenocarcinoma, pancreatic adenocarcinoma and non-seminomal testis carcinoma tissues. The epitope of the invention is predominantly present in filamentous cytoplasmic structures of these types of cells during proliferation. Testing indicates that the epitope is not detected in certain sarcomas, malignant melanomas, B-lymphomas or thymomas.

A sequence for human cytokeratin K8 is provided below (SEQ ID NO:1).

| | | | | |
|---|---|---|---|---|
| 1 | SIRVTQKSYK | VSTSGPRAFS | SRSYTSGPGS | RISSSSFSRV |
| 41 | GSSNFRGGLG | GGYGGASGMG | GITAVTVNQS | LLSPLSLEVD |
| 81 | PNIQAVRTQE | KEQIKTLNNK | FASFIDKVRF | LEQQNKMLET |
| 121 | KWSLLQQQKT | ARSNMDNMFE | SYINNLRRQL | ETLGQEKLKL |
| 161 | EAELGNMQGL | VEDFKNKYED | EINKRTEMEN | EFVLIKKDVD |
| 201 | EAYMNKVELE | SRLEGLTDEI | NFLRQLYEEE | IRELQSQISD |
| 241 | TSVVLSMDNS | RSLDMESIIA | EVKAQYEDIA | NRSRAEAESM |
| 281 | YQIKYEELQS | LAGKHGDDLR | RTKTEISEMN | RNISRLQAEI |
| 321 | EGLKGQRASL | EAAIADAEQR | GELAIKDANA | KLSELEAALQ |
| 361 | RAKQDMARQL | REYQELMNVK | LALDIDIATY | RKLLEGEESP |
| 401 | LESGMQNMSI | HTKTTGGYAG | GLSSAYGDLT | DPGLSYSLGS |
| 441 | SFGSGAGSSS | FSRTSSSRAV | VVKKIETRDG | KLVSESSDVL |
| 481 | PK | | | |

A nucleotide sequence for human cytokeratin K8 is provided below (SEQ ID NO:45).

| | | | | |
|---|---|---|---|---|
| 1 | TTCGGCAATT | CCTACCTCCA | CTCCTGCCTC | CACCATGTCC |
| 41 | ATCAGGGTGA | CCCAGAAGTC | CTACAAGGTG | TCCACCTCTG |
| 81 | GCCCCCGGGC | CTTCAGCAGC | CGCTCCTACA | CGAGTGGGCC |
| 121 | CGGTTCCCGC | ATCAGCTCCT | CGAGCTTCTC | CCGAGTGGGC |
| 161 | AGCAGCAACT | TTCGCGGTGG | CCTGGGCGGC | GGCTATGGTG |
| 201 | GGGCCAGCGG | CATGGGAGGC | ATCACCGCAG | TTACGGTCAA |
| 241 | CCAGAGCCTG | CTGAGCCCCT | TGTCCCTGGA | GGTGGACCCC |
| 281 | AACATCCAGG | CCGTGCGCAC | CCAGGAGAAG | GACCAGATCA |
| 321 | AGACCCTGAA | CAACAAGTTT | GCCTCCTTCA | TAGACAAGGT |
| 361 | ACGGTTCCTG | GAGCAGCAGA | ACAAGATGCT | GGAGACCAAG |
| 401 | TGGAGCCTCC | TGCAGCAGCA | GAAGACCGCT | CGAAGCAACA |
| 441 | TGGACAACAT | GTTCGAGAGC | TACATCAACA | ACCTTAGGCG |
| 481 | GCAGCTGGAG | ACTCTGGGCC | AGGAGAAGCT | GAAGCTGGAG |
| 521 | GCGGAGCTTG | GCAACATGCA | GGGGCTGGTG | GAGGACTTCA |
| 541 | AGAACAAGTA | TGAGCATGAG | ATCAATAAGC | GTACAGAGAT |
| 601 | GGAGAACGAA | TTTGTCCTCA | TCAAGAAGGA | TGTGGATGAA |
| 641 | GCATACATCA | ACAAGGTAGA | GCTGGAGTCT | CCCCTGGAAG |
| 681 | GGCTGACCGA | CGAGATCAAC | TTCCTCAGGC | AGCTGTATGA |
| 721 | AGAGGAGATC | CGGGAGCTGC | AGTCCCAGAT | CTCGGACACA |
| 761 | TCTGTGGTGC | TGTCCATCGA | CAACAGCCCC | TCCCTGGACA |
| 801 | TGGAGAGCAT | CATTGCTGAG | GTCAAGGCAC | AGTACGAGGA |
| 841 | TATTGCCAAC | CGCAGCCGGG | CTCAGGCTGA | GAGCATGTAC |
| 881 | CAGATCAAGT | ATCAGCAGCT | GCAGAGCCTC | GCTGGGAACC |
| 921 | ACGGGGATGA | CCTGCGGCGC | ACAAAGACTG | AGATCTCAGA |
| 961 | GATGAACCGG | AACATCAGCC | GGCTCCAGGC | TGAGATTGAG |
| 1001 | GGCCTCAAAG | GCCAGAGCGC | TTCCCTGGAG | GCCGCCATTG |
| 1041 | CAGATGCCGA | GCAGCGTGGA | GAGCTGGCCA | TTAAGGATGC |
| 1081 | CAACGCCAAG | TTGTCCGAGC | TGGAGGCCGC | CCTGCAGCGG |
| 1121 | GCCAAGCAGG | ACATGGCCCG | GCAGCTGCGT | GAGTACCAGG |
| 1161 | AGCTGATGAA | CGTCAAGCTG | GCCCTGGACA | TCGACATCGC |
| 1201 | CACCTACAGG | AAGCTGCTGG | ACGGCGAGGA | GAGCCCGCTG |
| 1241 | GAGTCTGGGA | TGCAGAACAT | GAGTATTCAT | ACGAAGACCA |
| 1281 | CCGGCGGCTA | TGCGGGTGGT | TTGAGCTCGG | CCTATGGGGA |
| 1321 | CCTCACAGAC | CCCGGCCTCA | GCTACAGCCT | GGGCTCCAGC |
| 1361 | TTTGGCTCTG | GCGCGGGCTC | CAGCTCCTTC | AGCCGCACCA |
| 1401 | GCTCCTCCAG | GGCCGTGGTT | GTGAAGAAGA | TCGAGACACG |
| 1441 | TGATGGGAAG | CTGGTGTCTG | AGTCCTCTGA | CGTCCTGCCC |
| 1481 | AAGTGAACAG | CTGCGGCAGC | CCCTCCCAGC | CTACCCCTCC |
| 1521 | TGCGCTGCCC | CAGAGCCTGG | GAAGGAGGCC | GCTATGCAGG |
| 1561 | GTAGCACTGG | GAACAGGAGA | CCCACCTGAG | GCTCAGCCCT |
| 1601 | AGCCCTCAGC | CCACCTGGGG | AGTTTACTAC | CTGGGGACCC |
| 1641 | CCCTTGCCCA | TGCCTCCAGC | TACAAAACAA | TTCAATTGCT |
| 1681 | TTTTTTTTTT | TTGGTCCCAA | AATAAAACCT | CAGCTAGCTC |
| 1721 | TGCC | | | |

A sequence for human cytokeratin K18 is provided below (SEQ ID NO:2).

| | | | | |
|---|---|---|---|---|
| 1 | SFTTRSTFST | NYRSLGSVQA | PSYGARPVSS | AASVYAGAGG |
| 41 | SGSRISVSRS | TSFRGGMGSG | GLATGIAGGL | AGMGGIQNEK |
| 81 | ETMQSLNDRL | ASYLDRVRSL | ETENRRLESK | IREHLEKKGP |
| 121 | QVRDWSHYFK | IIEDLRAQIF | ANTVDNARIV | LQIDNARLAA |
| 161 | DDFRVKYETE | LAMRQSVEND | IHGLRKVIDD | TNITRLQLET |
| 201 | EIEALKEELL | FMKKNHEEEV | KGLQAQIASS | GLTVEVDAPK |
| 241 | SQDLAKIMAD | IRAQYDELAR | KNREELDKYW | SQQIEESTTV |
| 281 | VTTQSAEVGA | AETTLTELRR | TVQSLEIDLD | SMRNLKASLE |
| 321 | NSLREVEARY | ALQMEQLNGI | LLHLESELAQ | TRAEGQRQAQ |
| 361 | EYEALLNIKV | KLEAEIATYR | RLLEDGEDFN | LGDALDSSNS |
| 401 | MQTIQKTTTR | RIVDGKVVSE | TNDTKVLRH | |

A nucleotide sequence for human cytokeratin K18 is provided below (SEQ ID NO:46).

| | | | | |
|---|---|---|---|---|
| 1 | CGGGGTCGTC | CGCAAAGCCT | GAGTCCTGTC | CTTTCTCTCT |
| 41 | CCCCGGACAG | CATGAGCTTC | ACCACTCGCT | CCACCTTCTC |
| 81 | CACCAACTAC | CGGTCCCTGG | GCTCTGTCCA | GGCGCCCAGC |
| 121 | TACGGCGCCC | GGCCGGTCAG | CAGCGCGGCC | AGCGTCTATG |
| 161 | CAGGCGCTGG | GGGCTCTGGT | TCCCGGATCT | CCGTGTCCCG |
| 201 | CTCCACCAGC | TTCAGGGGCG | GCATGGGGTC | CGGGGGCCTG |
| 241 | GCCACCGGGA | TAGCCGGGGG | TCTGGCAGGA | ATGGGAGGCA |
| 281 | TCCAGAACGA | GAAGGAGACC | ATGCAAAGCC | TGAACGACCG |
| 321 | CCTGGCCTCT | TACCTGGACA | GAGTGAGGAG | CCTGGAGACC |
| 361 | GAGAACCGGA | GGCTGGAGAG | CAAAATCCGG | GAGCACTTGG |
| 401 | AGAAGAAGGG | ACCCCAGGTC | AGAGACTGGA | GCCATTACTT |
| 441 | CAAGATCATC | GAGGACCTGA | GGGCTCAGAT | CTTCGCAAAT |
| 481 | ACTGTGGACA | ATGCCCGCAT | CGTTCTGCAG | ATTGACAATG |
| 521 | CCCGTCTTGC | TGCTGATGAC | TTTAGAGTCA | AGTATGAGAC |
| 561 | AGAGCTGGCC | ATGCGCCAGT | CTGTGGAGAA | CGACATCCAT |
| 601 | GGGCTCCGCA | AGGTCATTGA | TGACACCAAT | ATCACACGAC |
| 641 | TGCAGCTGGA | GACAGAGATC | GAGGCTCTCA | AGGAGGAGCT |
| 681 | GCTCTTCATG | AAGAAGAACC | ACGAAGAGGA | AGTAAAAGGC |
| 721 | CTACAAGCCC | AGATTGCCAG | CTCTGGGTTG | ACCGTGGAGG |
| 761 | TAGATGCCCC | CAAATCTCAG | GACCTCGCCA | AGATCATGGC |
| 801 | AGACATCCGG | GCCCAATATG | ACGAGCTGGC | TCGGAAGAAC |
| 841 | CGAGAGGAGC | TAGACAAGTA | CTGGTCTCAG | CAGATTGAGG |
| 881 | AGAGCACCAC | AGTGGTCACC | ACACAGTCTG | CTGAGGTTGG |
| 921 | AGCTGCTGAG | ACGACGCTCA | CAGAGCTGAG | ACGTACAGTC |
| 961 | CAGTCCTTGG | AGATCGACCT | GGACTCCATG | AGAAATCTGA |
| 1001 | AGGCCAGCTT | GGAGAACAGC | CTGAGGGAGG | TGGAGGCCCG |
| 1041 | CTACGCCCTA | CAGATGGAGC | AGCTCAACGG | GATCCTGCTG |
| 1081 | CACCTTGAGT | CAGAGCTGGC | ACAGACCCGG | GCAGAGGGAC |
| 1121 | AGCGCCAGGC | CCAGGAGTAT | GAGGCCCTGC | TGAACATCAA |
| 1161 | GGTCAAGCTG | GAGGCTGAGA | TCGCCACCTA | CCGCCGCCTG |
| 1201 | CTCGAAGATG | GCGAGGACTT | TAATCTTGGT | GATGCCTTGG |
| 1241 | ACAGCAGCAA | CTCCATGCAA | ACCATCCAAA | AGACCACCAC |
| 1281 | CCGCCGGATA | GTGGATGGCA | AAGTGGTGTC | TGAGACCAAT |
| 1321 | GACACCAAAG | TTCTGAGGCA | TTAAGCCAGC | AGAAGCAGGG |
| 1361 | TACCCTTTGG | GGAGCAGGAG | GCCAATAAAA | AGTTCAGAGT |
| 1401 | TCATTGGATG | TC | | |

The epitopes of the invention consist of two polypeptides, a cytokeratin K8 polypeptide and a cytokeratin K18 polypeptide. However, the cytokeratin K8 polypeptide is shorter than the full-length cytokeratin K8 polypeptide that has 482 amino acids. Moreover, the cytokeratin K18 polypeptide is shorter than the full-length cytokeratin K18 polypeptide that has 429 amino acids. In some embodiments, the cytokeratin K8 polypeptide is shorter than about 475 amino acids, or shorter than about 450 amino acids, or shorter than about 425 amino acids, or shorter than about 400 amino acids. In some embodiments, the cytokeratin K18 polypeptide is shorter than about 425 amino acids, or shorter than about 415 amino acids, or shorter than about 400 amino acids, or shorter than about 375 amino acids.

One example of an epitope of the invention constitutes two peptidyl regions of two separate proteins, cytokeratin K8 (SEQ ID NO:3) and cytokeratin K18 (SEQ ID NO:3). The epitope involves amino acids 85-129 of cytokeratin 8 sequence, designated SEQ ID NO:3 and provided below.

| | | | | |
|---|---|---|---|---|
| 1 | AVRTQEKEQI | KTLNNKFASF | IDKVRFLEQQ | NKMLETKWSL |
| 41 | LQQQ | | | |

The epitope further involves amino acids 72-124 of cytokeratin 18, designated SEQ ID NO:4 and provided below.

| | | | | |
|---|---|---|---|---|
| 1 | AGMGGIQNEK | ETMQSLNDRL | ASYLDRVRSL | ETENRRLESK |
| 41 | IREHLEKKGP | QVR | | |

In some instances an appropriate three dimensional structure permitting interaction between cytokeratin K8 and cytokeratin K18 polypeptides may be needed to obtain optimal immunoreactivity. Hence, longer cytokeratin polypeptides can be used as antigens. For example, a cytokeratin K8 polypeptide having SEQ ID NO:5 can be used with an appropriate cytokeratin K18 polypeptide to generate antibodies. SEQ ID NO:5 is as follows.

| | | | | |
|---|---|---|---|---|
| 84 | | | AVRTQE | KEQIKTLNNK |
| 101 | FASFIDKVRF | LEQQNKMLET | KWSLLQQQKT | ARSNMDNMFE |
| 141 | SYINNLRRQL | ETLGQEKLKL | EAELGNMQGL | VEDFKNKYED |
| 181 | EINKRTEMEN | EFVLIKKDVD | EAYMNKVELE | SRLEGLTDEI |
| 221 | NFLRQLYEEE | IRELQSQISD | TSVVLSMDNS | RSLDMESIIA |
| 261 | EVKAQYEDIA | NRSRAEAESM | YQIKYEELQS | LAGKHGDDLR |
| 301 | RTKTEISEMN | RNISRLQAEI | EGLKGQRASL | EAAIADAEQR |
| 341 | GELAIKDANA | KLSELEAALQ | RAKQDMARQL | REYQELMNVK |
| 381 | LALDIDIATY | RKLLEGEESP | LESGMQNMSI | HTKTTGGYAG |
| 421 | GLSSAYGDLT | DPGLSYSLGS | SFGSGAGSSS | FSRTSSSRAV |
| 461 | VVKKIETRDG | KLVSESSDVL | PK | |

Similarly, a cytokeratin K18 polypeptide having SEQ ID NO:6 can be used with an appropriate cytokeratin K8 polypeptide to generate antibodies. SEQ ID NO:6 is as follows.

| | | | | |
|---|---|---|---|---|
| 71 | | AGMGGIQNEK | ETMQSLNDRL | ASYLDRVRSL |
| 101 | ETENRRLESK | IREHLEKKGP | QVRDWSHYFK | IIEDLRAQIF |
| 141 | ANTVDNARIV | LQIDNARLAA | DDFRVKYETE | LAMRQSVEND |
| 181 | IHGLRKVIDD | TNITRLQLET | EIEALKEELL | FMKKNHEEEV |
| 221 | KGLQAQIASS | GLTVEVDAPK | SQDLAKIMAD | IRAQYDELAR |
| 261 | KNREELDKYW | SQQIEESTTV | VTTQSAEVGA | AETTLTELRR |
| 301 | TVQSLEIDLD | SMRNLKASLE | NSLREVEARY | ALQMEQLNGI |
| 341 | LLHLESELAQ | TRAEGQRQAQ | EYEALLNIKV | KLEAEIATYR |
| 381 | RLLEDGEDFN | LGDALDSSNS | MQTIQKTTTR | RIVDGKVVSE |
| 421 | TNDTKVLRH | | | |

Antigenic epitope “fragments” are also contemplated by the invention. Such fragments do not encompass a full-length cytokeratin but do encode an antigen that has similar or improved immunological properties relative to an antigenic epitope having SEQ ID NO:3-6. Thus, fragments of antigenic epitopes such as SEQ ID NO:3-6 may be as small as about 6 amino acids, about 9 amino acids, about 12 amino acids, about 15 amino acids, about 17 amino acids, about 18 amino acids, about 20 amino acids, about 25 amino acids, about 30 amino acids or more. In general, a fragment antigenic epitope of the invention can have any upper size limit so long as it is has similar or immunological properties relative to an epitope form by a combination of any one of SEQ ID NO:3-6.

The invention also contemplates a fusion protein comprising a combination of the SEQ ID NO:3 and the SEQ ID NO:4 peptide. Such a fusion protein links the two peptides together so that the peptides can more easily form the cancer associated epitope of the invention.

Fusion polypeptides may generally be prepared using standard techniques, including chemical conjugation. A fusion polypeptide can also expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A linker sequence may be employed to separate the first and second polypeptide components by a distance sufficient to ensure that each polypeptide folds into its secondary and tertiary structures. Such a linker can be a peptide, polypeptide, alkyl chain or other convenient spacer molecule.

A polypeptide or peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In some embodiments, peptide linker sequences contain Gly, Asn and Ser residues. Other near neutral amino acids, such as Thr and Ala may also be used in the linker sequence. Amino acid sequences that may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are generally not required when the first and second polypeptides have non-essential N-terminal amino acid regions that can be used to separate the functional domains and prevent steric interference.

The fusion polypeptide can comprise the polypeptide epitope (e.g. SEQ ID NO:3 and SEQ ID NO:4 peptides) as described herein together with an unrelated immunogenic protein, such as an immunogenic protein capable of eliciting a recall response. Examples of such proteins include tetanus, tuberculosis and hepatitis proteins (see, for example, Stoute et al. New Engl. J Med., 336:86-91, 1997).

In one embodiment, a peptide or polypeptide that can facilitate development of an immune response against the SEQ ID NO:3 and SEQ ID NO:4 peptide epitope is used as the linker. Such an immunological fusion partner can be derived from a Mycobacterium sp. For example, the immunological fusion partner can be a Mycobacterium tuberculosis-derived Ral2 fragment. Ral2 compositions and methods for their use in enhancing the expression and/or immunogenicity of heterologous polynucleotide/polypeptide sequences is described in U.S. patent application Ser. No. 60/158,585, the disclosure of which is incorporated herein by reference in its entirety. Briefly, Ral2 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. patent application Ser. No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ral2 may enhance the immunogenicity of heterologous immunogenic polypeptides with which it is fused. One useful Ral2 fusion polypeptide comprises a 14 KD C-terminal fragment corresponding to amino acid residues 192 to 323 of MTB32A. Other useful Ral2 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ral2 polypeptide.

Ral2 polynucleotides may comprise a native sequence (i.e., an endogenous sequence that encodes a Ral2 polypeptide or a portion thereof) or may comprise a variant of such a sequence. Ral2 polynucleotide variants may contain one or more substitutions, additions, deletions and/or insertions such that the biological activity of the encoded fusion polypeptide is not substantially diminished, relative to a fusion polypeptide comprising a native Ral2 polypeptide. Variants preferably exhibit at least about 70% identity, more preferably at least about 80% identity and most preferably at least about 90% identity to a polynucleotide sequence that encodes a native Ral2 polypeptide or a portion thereof.

In another embodiment, an immunological fusion partner is derived from protein D, a surface protein of the gram-negative bacterium Haemophilus influenza B (WO 91/18926). Useful portions of protein D comprise approximately the First third of the protein (e.g., the first N-terminal 100-110 amino acids). Moreover, such a protein D fusion partner may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LYTA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4.sup.+T-cells specific for the polypeptide.

Polypeptides and fusion proteins of the invention are prepared using any of a variety of well-known synthetic and/or recombinant techniques. Polypeptides and fusion proteins that are less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Small and large fusion proteins and polypeptide epitopes of the invention can be produced by any other method available to one of skill in the art. For example, the fusion proteins and polypeptide epitopes can be made recombinantly by inserting a nucleic acid encoding a selected fusion protein or polypeptide epitope into an expression vector using any of a variety of procedures. In general, a nucleic acid encoding the desired protein or polypeptide is inserted into an appropriate restriction endonuclease site(s) using techniques known in the art. See generally, Sambrook et al., 1989, Molecular Cloning, A Laboratory Manual, 2d ed., Cold Spring Harbor Laboratory Press, Cold Spring Harbor, N.Y.; Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)). Construction of suitable expression vectors containing a fusion protein or a polypeptide epitope employs standard ligation techniques that are known to the skilled artisan.

The ligated nucleic acid sequences are operably linked to suitable transcriptional or translational regulatory elements that facilitate expression of the fusion proteins and polypeptide epitopes of the invention. The regulatory elements responsible for expression of proteins are located only 5′ to the coding region for the polypeptide. Similarly, stop codons required to end translation and transcription termination signals are only present 3′ to the nucleic acid sequence encoding the fusion protein or polypeptide epitope. After construction of a nucleic acid encoding the polypeptide of interest with the operably linked regulatory elements, this expression cassette can be introduced into a host cell and the encoded polypeptide can be expressed.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Such polypeptides can also be purified. For example, the polypeptide epitopes and fusion proteins can be at least about 90% pure, or at least about 95% pure or at least about 99% pure.

Antibodies and Binding Entities

The cytokeratin epitopes of the invention are displayed in a uniform punctate pattern on the surface of viable carcinoma and adenocarcinoma cells. Immunohistological studies have demonstrated that the cancer associated epitope of the invention, in contrast to normal cytokeratin 8 and 18, can be used to differentiate between malignant and normal colon epithelia, and between colon cancer metastasis in the liver and surrounding normal hepatocytes. In addition, the cancer associated epitope of the invention is associated with the membranes of proliferating cells within the malignant area of biopsies, while resting cells had a filamentous pattern when stained for the epitope.

The invention provides antibody preparations and binding entities directed against the epitopes of the invention, for example, antibodies or binding entities capable of binding an antigenic mixture of at least one peptide from cytokeratin K8 and at least one peptide from cytokeratin k18. Examples of peptides from cytokeratin K8 include SEQ ID NO:3 and SEQ ID NO:5. Examples of peptides from cytokeratin K18 include SEQ ID NO:4, and SEQ ID NO:6.

In one embodiment, the antibody or binding entity can include a polypeptide comprising any one of SEQ If) NO:7-35, 47-49. In some embodiments, antibodies and binding entities include a polypeptide consisting essentially of any one of SEQ ID NO:21-35, 47-49. In other embodiments, antibodies and binding entities include a polypeptide consisting essentially of any one of SEQ ID NO:8, 10, 12, 15, 17, 19, 22, 24, 27, 29 or 32. In another embodiment, the invention is directed to a binding entity polypeptide comprising any combination of SEQ ID NO:7-33, 47-49, wherein the polypeptide that can bind an epitope of the invention.

The invention also provides nucleic acids encoding antibody-like polypeptides of the invention. In one embodiment, the nucleic acid encodes a polypeptide comprising any one of SEQ ID NO:7-35, 47-49 wherein such a nucleic acid encodes a polypeptide that can bind an epitope of the invention. In another embodiment, the nucleic acid encodes a combination of two or more of SEQ ID NO:7-33, 47-49 wherein such a nucleic acid encodes a binding entity polypeptide that can bind an epitope of the invention. Preferred nucleic acids encode a polypeptide consisting essentially of any one of SEQ ID NO:21-33 or any one of SEQ ID NO:8, 10, 12, 15, 17, 19, 22, 24, 27, 29 or 32. Other nucleic acids of the invention include nucleotide sequences SEQ ID NO:36-39.

The invention also provides antibodies made by available procedures that can bind an epitope of the invention.

Antibody molecules belong to a family of plasma proteins called immunoglobulins, whose basic building block, the immunoglobulin fold or domain, is used in various forms in many molecules of the immune system and other biological recognition systems. A standard antibody is a tetrameric structure consisting of two identical immunoglobulin heavy chains and two identical light chains and has a molecular weight of about 150,000 daltons.

The heavy and light chains of an antibody consist of different domains. Each light chain has one variable domain (VL) and one constant domain (CL), while each heavy chain has one variable domain (VH) and three or four constant domains (CH). See, e.g., Alzari, P. N., Lascombe, M. B. & Poljak, R. J. (1988) Three-dimensional structure of antibodies. Annu. Rev. Immunol. 6, 555-580. Each domain, consisting of about 110 amino acid residues, is folded into a characteristic β-sandwich structure formed from two β-sheets packed against each other, the immunoglobulin fold. The VH and VL domains each have three complementarity determining regions (CDR1-3) that are loops, or turns, connecting β-strands at one end of the domains. The variable regions of both the light and heavy chains generally contribute to antigen specificity, although the contribution of the individual chains to specificity is not always equal. Antibody molecules have evolved to bind to a large number of molecules by using six randomized loops (CDRs).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (X), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention. In some embodiments, the antibody is not the COU-1 antibody.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments contemplated by the invention are therefore not full-length antibodies but do have similar or improved immunological properties relative to an antibody such as the COU-1 antibody. Thus, fragments of the COU-1 antibody and/or fragments of polypeptides having any one of SEQ ID NO:7-35 antibody are contemplated by the invention. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more.

In general, an antibody fragment of the invention can have any upper size limit so long as it is has similar or immunological properties relative to antibody that binds with specificity to an epitope formed by a combination of any one of SEQ ID NO:3-6. Such a reference antibody can be the COU-1 antibody. For example, binding entities and light chain antibody fragments can have less than about 200 amino acids, less than about 175 amino acids, less than about 150 amino acids, or less than about 120 amino acids if the antibody fragment is related to a light chain antibody subunit. Moreover, binding entities and heavy chain antibody fragments can have less than about 425 amino acids, less than about 400 amino acids, less than about 375 amino acids, less than about 350 amino acids, less than about 325 amino acids or less than about 300 amino acids if the antibody fragment is related to a heavy chain antibody subunit.

Antibody fragments retain some ability to selectively bind with its antigen, epitope or receptor. Some types of antibody fragments are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

Methods for the preparation of polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of monoclonal antibodies are well known to those skilled in the art. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol Biol. 222: 581-597 (1991). Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et at. Proc. Natl. Acad Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11: 1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

While standardized procedures are available to generate antibodies, the size of antibodies, the multi-stranded structure of antibodies and the complexity of six binding loops present in antibodies constitute a hurdle to the improvement and the manufacture of large quantities of antibodies. Hence, the invention further contemplates using binding entities, which comprise polypeptides that can recognize and bind to the epitope of the invention.

The invention is therefore further directed to antibodies and other binding entities that can bind the cancer-associated epitope of the invention. In some embodiments, the antibodies and binding entities have SEQ ID NO:7-33. The sequences for SEQ ID NO:7-33 are provided below.

| | | |
|---|---|---|
| SEQ ID NO: | Sequence | Ab Region |

| | | |
|---|---|---|
| SEQ ID NO:7 | GAEVKKPGASVKVSCKASDYTFS | VH FR1 |
| SEQ ID NO:8 | SYYMH | VH CDR1 |
| SEQ ID NO:9 | WVRQAPGQGLEWMG | VHFR2 |
| SEQ ID NO:10 | IINPSGGSTSYAQKFQG | VH CDR2 |
| SEQ ID NO:11 | RVTMTRDTSTNTVYMELSSLRSE | VH FR3 |
| | DTAVYYCAR | |
| SEQ ID NO:12 | DQVVVAATLSNYGMDV | VH CDR3 |
| SEQ ID NO:13 | WGQGTTVTVSSAST | VH FR4 |
| SEQ ID NO:14 | ELTQSPGTLSLSPGERATLSC | VL FR1 |
| SEQ ID NO:15 | RASQSVSSSYLA | VL CDR1 |
| SEQ ID NO:16 | WYQQKPGQAPRLLIY | VL FR2 |
| SEQ ID NO:17 | DASNRAT | VL CDR2 |
| SEQ ID NO:18 | GIPARFSGSGSGTDFTLTISS | VL FR3 |
| | LEPEDFAVYYC | |
| SEQ ID NO:19 | QQGTNWGIA | VL CDR3 |
| SEQ ID NO:20 | FGQGTRLDIKR | VL FR4 |
| SEQ ID NO:21 | TQSPGTLSLSPGERATLSC | VL FR1 |
| SEQ ID NO:22 | GASSRAT | VL CDR2 |
| SEQ ID NO:23 | GIPDRFSGSGSGTDFTLTI | VL FR3 |
| | SRLEPEDFAAYYC | |
| SEQ ID NO:24 | QQYGNSPPYT | VL CDR3 |
| SEQ ID NO:25 | FGQGTKLEI | VL FR4 |
| SEQ ID NO:26 | TQSPDSLAVSLGERATINC | VL FR1 |
| SEQ ID NO:27 | KSSQSLLYSSNNKNYLA | VL CDR1 |
| SEQ ID NO:28 | WYQQKPGQPPKLLIY | VL FR2 |
| SEQ ID NO:29 | WASTRES | VL CDR2 |
| SEQ ID NO:30 | GVPDRFSGSGSGT | VL FR3 |
| SEQ ID NO:31 | DFTLTISSLQAEDVAGYYC | VL FR3 |
| SEQ ID NO:32 | QQYYSTPPM | VL CDR3 |
| SEQ ID NO:33 | FGQGTKVEI | VL FR4 |

Nucleic acids encoding peptides SEQ ID NO:7-33 were isolated from cells that secrete the COU-1 antibody. While not all of the polypeptides encoded by the nucleic acids isolated in this screen could bind the cancer-associated epitope, peptides SEQ ID NO:7-33 were shown to play a role in binding by phage display and other experiments. Moreover, several differences were found in similar regions of different antibody fragment clones. For example, variable light chain CDR1 fragments that were isolated had RASQSVSSSYLA (SEQ ID NO:15) as well as KSSQSLLYSSNNKNYLA (SEQ ID NO:27). Similarly, variable light chain CDR2 fragments isolated had DASNRAT (SEQ ID NO:17), GASSRAT (SEQ ID NO:22) or WASTRES (SEQ ID NO:29). Moreover, variable light chain CDR3 fragments isolated had QQYGNSPPYT (SEQ ID NO:24) or QQYYSTPPM (SEQ ID NO:32). Hence, not all clones were identical.

A number of proteins can serve as protein scaffolds to which binding domains (e.g. any of the SEQ ID NO:7-33, 47-49 peptides or variants thereof) can be attached. The binding domains bind or interact with the cancer-associated epitope of the invention while the protein scaffold merely holds and stabilizes the binding domains so that they can bind. A number of protein scaffolds can be used. For example, phage capsid proteins can be used. Review in Clackson & Wells, Trends Biotechnol. 12:173-184 (1994). Indeed, such phage capsid proteins were used as described herein to screen for the SEQ ID NO:7-33 peptides (see Examples). Phage capsid proteins have also been used as scaffolds for displaying random peptide sequences, including bovine pancreatic trypsin inhibitor (Roberts et al., PNAS 89:2429-2433 (1992)), human growth hormone (Lowman et al., Biochemistry 30:10832-10838 (1991)), Venturini et al., Protein Peptide Letters 1:70-75 (1994)), and the IgG binding domain of Streptococcus (O'Neil et al., Techniques in Protein Chemistry V (Crabb, L,. ed.) pp. 517-524, Academic Press, San Diego (1994)). These scaffolds have displayed a single randomized loop or region that can be modified to include the binding domains provided herein (e.g. SEQ ID NO:7-33, 47-49).

Researchers have also used the small 74 amino acid α-amylase inhibitor Tendamistat as a presentation scaffold on the filamentous phage M13. McConnell, S. J., & Hoess, R. H., J. Mol. Biol. 250:460-470 (1995). Tendamistat is a β-sheet protein from Streptomyces tendae. It has a number of features that make it an attractive scaffold for binding peptides, including its small size, stability, and the availability of high resolution NMR and X-ray structural data. The overall topology of Tendamistat is similar to that of an immunoglobulin domain, with two β-sheets connected by a series of loops. In contrast to immunoglobulin domains, the β-sheets of Tendamistat are held together with two rather than one disulfide bond, accounting for the considerable stability of the protein. By analogy with the CDR loops found in immunoglobulins, the loops of Tendamistat may serve a similar function and can be easily randomized by in vitro mutagenesis. Tendamistat, however, is derived from Streptomyces tendae and may be antigenic in humans. Its small size, however, may reduce or inhibit its antigenicity.

Fibronectin type III domain has also been used as a protein scaffold to which binding entities can be attached. Sequences, vectors and cloning procedures for using such a fibronectin type III domain as a protein scaffold for binding entities (e.g. CDR peptides) are provided, for example, in U.S. patent application Publication 20020019517. Fibronectin is a large protein that plays an essential role in the formation of extracellular matrix and cell-cell interactions. Fibronectin consists of many repeats of three types (I, II and III) of small domains. Baron, M., Norman, D. G. & Campbell, I. D. (199 1) Protein modules Trends Biochem. Sci. 16, 13-17. Fibronectin type III is part of a large subfamily (Fn3 family or s-type Ig family) of the immunoglobulin superfamily. The Fn3 family includes cell adhesion molecules, cell surface hormone and cytokine receptors, chaperoning, and carbohydrate-binding domains. For reviews, see Bork, P. & Doolittle, R. F. (1992) Proposed acquisition of an animal protein domain by bacteria. Proc. Natl. Acad. Sci. USA 89, 8990-8994; Jones, E. Y. (1993) The immunoglobulin superfamily Curr. Opinion Struct. Biol. 3, 846-852; Bork, P., Hom, L. & Sander, C. (1994) The immunoglobulin fold. Structural classification, sequence patterns and common core. J. Mol. Biol. 242, 309-320; Campbell, I. D. & Spitzfaden, C. (1994) Building proteins with fibronectin type III modules Structure 2, 233-337; Harpez, Y. & Chothia, C. (1994) Many of the immunoglobulin superfamily domains in cell adhesion molecules and surface receptors belong to a new structural set which is close to that containing variable domains J. Mol. Biol. 238, 528-539.

In the immune system, specific antibodies are selected and amplified from a large library (affinity maturation). The combinatorial techniques employed in immune cells can be mimicked by mutagenesis and generation of combinatorial libraries of binding entities. Binding entities, antibody fragments and antibodies therefore can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No.5,733,743.

The invention therefore also provides methods of mutating antibodies to optimize their affinity, selectivity, binding strength and/or other desirable properties. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody. One method of mutating antibodies involves affinity maturation using phage display.

For example, affinity maturation using phage display can be utilized as one method for generating mutant antibodies. Affinity maturation using phage display refers to a process described in Lowman et al., Biochemistry 30(45): 10832-10838 (1991), see also Hawkins et al., J. Mol Biol. 254: 889-896 (1992). While not strictly limited to the following description, this process can be described briefly as involving mutation of several antibody hypervariable regions in a number of different sites with the goal of generating all possible amino acid substitutions at each site. The antibody mutants thus generated are displayed in a monovalent fashion from filamentous phage particles as fusion proteins. Fusions are generally made to the gene III product of M13. The phage expressing the various mutants can be cycled through several rounds of selection for the trait of interest, e.g. binding affinity or selectivity. The mutants of interest are isolated and sequenced. Such methods are described in more detail in U.S. Pat. No. 5,750,373, U.S. Pat. No. 6,290,957 and Cunningham, B. C. et al., EMBO J. 13(11), 2508-2515 (1994).

In one embodiment, the invention provides methods of manipulating antibody polypeptides or antibody-encoding nucleic acids to generate antibodies and antibody fragments with improved binding properties that recognize the same epitope as COU-1 antibodies.

Such methods of mutating portions of a COU-1 antibody involve fusing a nucleic acid encoding a polypeptide having any one of SEQ ID NO:7-35 or any one of SEQ ID NO:8, 10, 12, 15, 17, 19, 22, 24, 27, 29, 32, 47, 48 or 49 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention.

In one embodiment, the method involves fusing a nucleic acid encoding a polypeptide having any combination of SEQ ID NO:7-35 or any combination of SEQ ID NO:8, 10, 12, 15, 17, 19, 22, 24, 27, 29, 32, 47, 48 or 49 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention.

In another embodiment, the method involves fusing a nucleic acid encoding a polypeptide having each one of SEQ ID NO:26, 15, 27, 22, 23, 24 and 25 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention.

In another embodiment, the method involves fusing a nucleic acid encoding a polypeptide having each one of SEQ ID NO:26, 27, 28, 29, 30, 31, 32 and 33 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention.

In another embodiment, the method involves fusing a nucleic acid encoding a polypeptide having SEQ ID NO:34 or SEQ ID NO:35 to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention. SEQ ID NO:34 and 35 encode useful variable light chains that may bind to epitopes of the invention. SEQ ID NO:34 is provided below.

TQSPGTLSLSPGERATLSCRASQSVSSSYLAWYQQKPGQAPRLLIYGASS
RATGIPDRFSGSGSGTDFTLTISRLEPEDFAAYYCQQYGNSPPYTFGQGT
KLEI
SEQ ID NO:35 is provided below.
TQSPDSLAVSLGERATINCKSSQSLLYSSNNKNYLAWYQQKPGQPPKLLI
YWASTRESGVPDRFSGSGSGTDFTLTISSLQAEDVAGYYCQQYYSTPPMF
GQGTKVEI

The method can also involve fusing a nucleic acid comprising a variable heavy or light chain relating to COU-1 (e.g. any one of SEQ ID NO:36-39) to a nucleic acid encoding a phage coat protein to generate a recombinant nucleic acid encoding a fusion protein, mutating the recombinant nucleic acid encoding the fusion protein to generate a mutant nucleic acid encoding a mutant fusion protein, expressing the mutant fusion protein on the surface of a phage and selecting phage that bind to an epitope of the invention.

Hence, the invention is directed to a nucleic acid encoding a variable heavy chain relating to COU-1, for example, SEQ ID NO:36 provided below.

GGGGCTGAGGTGAAGAAGCCTGGGGCGTCAGTGAAGGTTTCCTGCAA
GGCATCTGGATACACCTTCAGCAGCTACTATATGCACTGGGTGCGAC
AGGCCCCTGGACAAGGGCTTGAGTGGATGGGAATAATCAACCCTAGT
GGTGGTAGCACAAGCTACGCACAGAAGTTCCAGGGCAGAGTCACCAT
GACCAGGGACACGTCCACGAACACAGTCTACATGGAGCTGAGCAGC
CTGAGATCTGAGGACACGGCCGTGTATTACTGTGCGAGAGATCAGGT
GGTGGTAGCTGCTACTTTGTCCAACTACGGTATGGACGTCTGGGGCC
AAGGGACCACGGTCACCGTCTCCTCA

In another embodiment the invention is directed to a nucleic acid encoding a variable light chain relating to COU-1, for example, SEQ ID NO:37 provided below.

GAGCTCACCCAGTCTCCAGGCACCCTGTCTTTGTCTCCAGGGGAAAGA
GCCACCCTCTCCTGCAGGGCCAGTCAGAGTGTTAGTAGCAGCTACTTA
GCCTGGTACCAGCAGAAACCTGGCCAGGCTCCCAGGCTCCTCATCTAT
GATGCATCCAACAGGGCCACTGGCATCCCAGCCAGGTTCAGTGGCAG
TGGGTCTGGGACAGACTTCACTCTCACCATCAGCAGCCTAGAGCCTGA
AGATTTTGCAGTTTATTACTGTCAGCAGGGTACCAACTGGGGGATCGC
CTTCGGCCAAGGGACACGACTGGATATTAAACGA

In another embodiment the invention is directed to a nucleic acid encoding a variable light chain relating to COU-1, for example, SEQ ID NO:38 (also called L8) provided below.

ACGCAGTCTCCAGGCACCCTGTCTTTTGTCTCCAGGGGAAAGAGCCACC
CTCTCCTGTAGGGCCAGTCAGAGTGTTAGCAGCAGCTACTTAGCCTGG
TACCAGCAGAAACCTGGCCAGGCTCCCAGGCTCCTCATCTATGGTGCA
TCCAGCAGGGCCACTGGCATCCCAGACAGGTTCAGTGGCAGTGGGTC
AGGGACAGACTTCACTCTCACCATCAGCAGACTGGAGCCTGAAGATTT
TGCAGCGTATTACTGTCAGCAGTATGGTAACTCACCTCCGTACACTTTT
GGCCAGGGGACCAAGCTGGAGATCA

In another embodiment the invention is directed to a nucleic acid encoding a variable light chain related to COU-1, for example, SEQ ID NO:39 (also called T5).

ACCCAGTCTCCAGACTCCCTGGCTGTGTCTCTGGGCGAGAGGGCCACC
ATCAACTGCAAGTCCAGCCAGAGTCTTTTATACAGCTCCAACAATAAG
AACTACTTAGCTTGGTACCAGCAGAAACCAGGACAGCCTCCTAAGTTG
CTCATTTACTGGGCATCTACCCGGGAATCCGGGGTCCCTGACCGATTC
AGTGGCAGCGGGTCTGGGACAGATTTCACTCTCACCATCAGCAGCCTG
CAGGCTGAAGATGTGGCAGGTTATTACTGTCAGCAATATTATAGTACT
CCTCCGATGTTCGGCCAAGGGACCAAGGTGGAAATC

Such methods can further include constructing a replicable expression vector containing a nucleic acid encoding a polypeptide of the invention, for example, a polypeptide comprising any one of SEQ ID NO:7-35, or a nucleic acid comprising any one of SEQ ID NO:36-39. The nucleic acid can also encode a fusion protein comprising a polypeptide of the invention (e.g. any of SEQ ID NO:7-35) and at least a portion of a natural or wild-type phage coat protein. The expression vector can also have a transcription regulatory element operably linked to the nucleic acids encoding the fusion protein. The vector is mutated at one or more selected positions within the nucleic acid encoding the antibody polypeptide to form a family or “library” of plasmids containing related nucleic acids, each encoding a slightly different antibody polypeptide. Suitable host cells are transformed with the family of plasmids. The transformed host cells are infected with a helper phage having a gene encoding the phage coat protein and the transformed, infected host cells are cultured under conditions suitable for forming recombinant phagemide particles. Each recombinant phagemid displays approximately one copy of the fusion protein on the surface of the phagemid particle. To screen the phagemids, phagemid particles are contacted with an epitope or antigen of the invention. Phagemid particles that bind are separated from those that do not bind the epitope or antigen. Preferably, further rounds of selection are performed by separately cloning phagemids with acceptable binding properties and re-testing their binding affinity one or more times. The plasmids from phagemid particles that appropriately bind the epitope or antigen can also be isolated, cloned and even mutated again to further select for the antibody properties desired, e.g. with good binding affinity.

The method is applicable to polypeptide complexes that are composed of more than one subunit polypeptides. In this case, a nucleic acid encoding each subunit of interest is separately fused to a phage coat protein and separately analyzed for its binding properties.

Any cloning procedure used by one of skill in the art can be employed to make the expression vectors used in such affinity maturation/phage display procedures. For example, one of skill in the art can readily employ known cloning procedures to fuse a nucleic acid encoding an antibody hypervariable region to a nucleic acid encoding a phage coat protein. See, e.g., Sambrook et al., Molecular Cloning, A Laboratory Manual, Cold Spring Harbor Laboratory, N.Y., 1989; Sambrook et al., Molecular Cloning, A Laboratory Manual, Cold Spring Harbor Laboratory, N.Y., 2001.

The invention is therefore directed to a method for selecting antibodies and/or antibody fragments or polypeptides with desirable properties. Such desirable properties can include increased binding affinity or selectivity for the epitopes of the invention.

The antibodies and antibody fragments of the invention are isolated antibodies and antibody fragments. An isolated antibody is one that has been identified and separated and/or recovered from a component of the environment in which it was produced. Contaminant components of its production environment are materials that would interfere with diagnostic or therapeutic uses for the antibody, and may include enzymes, hormones, and other proteinaceous or nonproteinaceous solutes. The term “isolated antibody” also includes antibodies within recombinant cells because at least one component of the antibody's natural environment will not be present. Ordinarily, however, isolated antibody will be prepared by at least one purification step

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In preferred embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequentator; or 3) to homogeneity by SDS-PAGE under reducing or non-reducing conditions using Coomasie blue or, preferably, silver stain.

Antigen, Binding Entity and Antibody Variants and Derivatives

The invention also provides variants and derivative of the antigenic epitopes, binding entities and antibody segments identified herein. For example, any derivative or variant of a SEQ ID NO:3, 4, 5 or 6 antigenic epitope is contemplated as being within the scope of the invention, particularly when the variant or derivative retains, or has improved, specificity as a vaccine for preventing or treating adenocarcinomas or is an improved marker for detecting adenocarcinomas. Similarly, any derivative or variant of a SEQ ID NO:7-35 antibody polypeptide is contemplated by the invention, particularly when the variant or derivative antibody polypeptide has improved specificity or binding affinity for an antigenic epitope of the invention, for example, an antigenic epitope having SEQ ID NO:3, 4, 5 or 6.

Derivative and variant antigenic epitopes and antibody segments of the invention are derived from the reference antigenic epitopes and antibody segments by deletion or addition of one or more amino acids to the N-terminal and/or C-terminal end of the reference antigenic epitopes and antibody segments; deletion or addition of one or more amino acids at one or more sites within the reference antigenic epitopes and antibody segments; or substitution of one or more amino acids at one or more sites within the reference antigenic epitopes and antibody segments. Thus, the antigenic epitopes and antibody segments of the invention may be altered in various ways including amino acid substitutions, deletions, truncations, and insertions.

Such variant and derivative antigenic epitopes and antibody segments may result, for example, from human manipulation. For example, the affinity maturation techniques using phage display described above may be used to generate variants and derivatives of both the antigenic epitopes and antibody segments of the invention. Other methods for mutating or altering the sequence of polypeptide are generally available in the art. For example, amino acid sequence variants of the antigenic epitopes and antibody segments can be prepared by mutations in the DNA encoding these antigenic epitopes and antibody segments. Methods for mutagenesis and nucleotide sequence alterations are also available in the art. See, for example, Kunkel, Proc. Natl. Acad. Sci. USA, 82, 488 (1985); Kunkel et al., Methods in Enzymol., 154, 367 (1987); U.S. Pat. No. 4,873,192; Walker and Gaastra, eds., Techniques in Molecular Biology, MacMillan Publishing Company, New York (1983) and the references cited therein. Guidance as to appropriate amino acid substitutions that do not adversely affect the structural integrity and/or biological activity of the peptide of interest may be found in the model of Dayhoff et al., Atlas of Protein Sequence and Structure, Natl. Biomed. Res. Found., Washington, C.D. (1978), herein incorporated by reference.

The derivatives and variants of the antigenic epitopes and antibody segments of the invention have identity with at least about 90%, 91%, 92%, 93% or 94% of the amino acid positions of any one of SEQ ID NO:3-35 and generally have similar or improved immunological properties relative to those of the antigenic epitopes and antibody segments having any one of SEQ ID NO:3-35. In a desirable embodiment, the antigenic epitopes and antibody segment derivatives and variants have identity with at least about 95% or 96% of the amino acid positions of any one of SEQ ID NO:3-35 and generally have immunological properties that are similar or better than the antigenic epitopes and antibody segments having SEQ ID NO:3-35. In a more desirable embodiment, the antigenic epitopes and antibody segments derivatives and variants have identity with at least about 97% or 98% of the amino acid positions of any one of SEQ IfD NO:3-35 and generally have similar or improved immunological properties relative to those of the antigenic epitopes and antibody segments having SEQ ID NO:3-35.

By “similar or improved immunological properties” is meant that a derivative or variant of a SEQ ID NO:3, 4, 5 or 6 antigenic epitope retains, or has improved, activity as a vaccine for preventing or treating adenocarcinomas or is an improved marker for detecting adenocarcinomas. Similarly, derivatives or variants of a SEQ ID NO:7-35 antibody polypeptide have “similar or improved immunological properties” when they have improved specificity or binding affinity for an antigenic epitope of the invention, for example, an antigenic epitope having SEQ ID NO:3, 4, 5 or 6.

Amino acid residues of the antigenic epitopes, binding entities and antibody segments and of the derivatives and variants thereof can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 1.

TABLE 1

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | Bala |
| 2,3-Diaminopropionic | | Dpr |
| acid | | |
| α-Aminoisobutyric | | Aib |
| acid | | |
| N-Methylglycine | | MeGly |
| (sarcosine) | | |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3- | | |
| carboxylic acid | | |
| β-2-thienylalanine | | Thi |
| Methionine sulfoxide | | MSO |
| Homoarginine | | Harg |
| N-acetyl lysine | | AcLys |
| 2,4-Diamino butyric | | Dbu |
| acid | | |
| ρ-Aminophenylalanine | | Phe(pNH₂) |
| N-methylvaline | | MeVal |
| Homocysteine | | Hcys |
| Homoserine | | Hser |
| ε-Amino hexanoic | | Aha |
| acid | | |
| δ-Amino valeric acid | | Ava |
| 2,3-Diaminobutyric | | Dab |
| acid | | |

Variants of the present antigenic epitopes and antibody segments that are encompassed within the scope of the invention can have one or more amino acids substituted with an amino acid of similar chemical and/or physical properties, so long as the backbone portions of these variant peptides have similar or improved immunological properties relative to those of antigenic epitopes and antibody segments having any one of SEQ ID NO:3-35. Derivative antigenic epitopes and antibody segments can have additional peptide or chemical moieties as well as one or more amino acids substituted with amino acids having different chemical and/or physical properties, so long as these derivative antigenic epitopes and antibody segments have similar or improved immunological properties relative to those of antigenic epitopes and antibody segments having any one of SEQ ID NO:3-35.

Amino acids that are substitutable for each other to form a variant antigenic epitopes and antibody segments of the invention generally reside within similar classes or subclasses. As known to one of skill in the art, amino acids can be placed into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include glycine, proline and methionine. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include cysteine. Examples of non-genetically encoded cysteine-like amino acids include homocysteine and penicillamine.

As will be appreciated by those having skill in the art, the above classifications are not absolute. Several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a polypeptide.

Certain commonly encountered amino acids that are not genetically encoded and that can be present, or substituted for an amino acid, in the variant polypeptides of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine (MeGly); ornithine (Om); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall into the categories defined above.

The classifications of the above-described genetically encoded and non-encoded amino acids are summarized in Table 2, below. It is to be understood that Table 2 is for illustrative purposes only and does not purport to be an exhaustive list of amino acid residues that may comprise the variant and derivative antigenic epitopes and antibody segments described herein. Other amino acid residues that are useful for making the variant and derivative polypeptides described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 2

| Classification | Genetically Encoded | Genetically Non-Encoded |
|---|---|---|
| Hydrophobic | F, L, I, V | |
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl |
| | | Ala |
| Apolar | M, G, P | |
| Aliphatic | A, V, L, I | t-BuA, t-BuG, MeIle, Nle, |
| | | MeVal, Cha, bAla, MeGly, |
| | | Aib |
| Hydrophilic | S, K | Cit, hCys |
| Acidic | D, E | |
| Basic | H, K, R | Dpr, Orn, hArg, Phe(p-NH₂), |
| | | DBU, A₂ BU |
| Polar | Q, N, S, T, Y | Cit, AcLys, MSO, hSer |
| Cysteine-Like | C | Pen, hCys, β-methyl Cys |

Antigenic epitopes and antibody segments of the invention can have any amino acid substituted by any similarly classified amino acid to create a variant antigenic epitope or a variant antibody segment, so long as the variant has similar or improved immunological properties relative to those of an antigenic epitope or antibody segment having any one of SEQ ID NO:3-35.

The invention is therefore also directed to binding entities and antibodies with binding domains related to the variable light or heavy chain CDR fragments isolated according to the invention. For example, the variable light chain CDR1 fragments can be aligned as follows:

| | |
|---|---|
| RASQS V-SSS ----Y LA | (SEQ ID NO:15) |
| KSSQS LLYSS NNKNY LA | (SEQ ID NO:27) |

Related variable light chain CDR1 fragments and binding entities are of the following formula (SEQ ID NO:47).

Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-
Xaa₂₁-Xaa₂₂-Xaa₂₃-Xaa₂₄-Xaa₂₅-Xaa₂₆-Xaa₂₇

wherein:
Xaa₁₁ is a basic amino acid;
Xaa₁₂ is an aliphatic amino acid or a polar amino acid;
Xaa₁₃, Xaa₁₅, Xaa₁₉, and Xaa₂₀ are separately each a serine;
Xaa₁₄ is a glutamine;
Xaa₁₆ Xaa₂₆, and Xaa₂₇ separately each an aliphatic amino acid;
Xaa₁₇ is an aliphatic amino acid or no amino acid;
Xaa₁₈ and Xaa₂₅ are separately each a polar amino acid;
Xaa₂₁, Xaa₂₂ and Xaa₂₄ are separately each a polar amino acid or no amino acid;
   and
Xaa₂₃ is a basic amino acid or no amino acid.

In some embodiments, Xaa₂₁, Xaa₂₂ and Xaa₂₄ are asparagine or no amino acid. In other embodiments, Xaa₂₅ is tyrosine.

The variable light chain CDR2 fragments can be aligned as follows:

| | |
|---|---|
| DASNRAT, | (SEQ ID NO:17) |
| GASSRAT | (SEQ ID NO:22) |
| and | |
| WASTRES. | (SEQ ID NO:29) |

Related variable light chain CDR2 fragments and binding entities are of the following formula (SEQ ID NO:48).

Xaa₃₁-Xaa₃₂-Xaa₃₃-Xaa₃₄-Xaa₃₅-Xaa₃₆-Xaa₃₇

wherein:
Xaa₃₁ is an acidic, apolar or aromatic amino acid;
Xaa₃₂ is an alanine;
Xaa₃₃ is a serine;
Xaa₃₄ and Xaa₃₇ separately each a polar amino acid;
Xaa₃₅ is a basic amino acid; and
Xaa₃₆ is an acidic or aliphatic amino acid.

The variable light chain CDR3 fragments can be aligned as follows:

| | |
|---|---|
| QQYGNSPPYT | (SEQ ID NO:24) |
| and | |
| QQYYSTPPM. | (SEQ ID NO:32) |

Related variable light chain CDR3 fragments and binding entities are of the following formula (SEQ ID NO:49).

Xaa₄₁-Xaa₄₂-Xaa₄₃-Xaa₄₄-Xaa₄₅-Xaa₄₆-Xaa₄₇-Xaa₄₈-
Xaa₄₉-Xaa₅₀

wherein:
Xaa41 and Xaa₄₂ separately are each a glutamine;
Xaa₄₃ is a tyrosine;
Xaa₄₄ and Xaa₄₉ separately are each an apolar, polar or aromatic amino acid;
Xaa₄₅ and Xaa₄₆ separately are each a polar amino acid;
Xaa₄₇ and Xaa₄₈ separately are each a proline; and
Xaa₅₀ is a polar amino acid or no amino acid.
   Detecting the Cancer-Associated Epitope

The invention also provides methods of detecting the cancer-associated epitopes of the invention in biological test samples. Any immunoassay or in vivo imaging procedure known to one of skill in the art can be used to detect the cancer-associated epitopes of the invention in a biological test sample. For example, the cancer-associated epitopes of the invention can be detected by immunochemical, immunohistological, ELISA, radioimmunoassay, nuclear magnetic resonance, magnetic resonance imaging, surface plasmon resonance and related procedures.

Such methods can include the steps of contacting a test sample with an antibody or binding entity capable of binding to a cancer-associated epitope of the invention, and determining whether the antibody or binding entity binds to a component of the sample. These methods can also include the steps of obtaining a biological sample (e.g., cells, blood, plasma, tissue, etc.) from a patient suspected of having cancer, contacting the sample with a labeled antibody or a labeled binding entity that is specific for the cancer-associated epitope of the invention, and detecting the epitope using standard immunoassay and/or diagnostic imaging techniques. Binding of the antibody or binding entity to the biological sample indicates that the sample contains the epitope.

In another embodiment, the cancer-associated epitope can be used to detect antibodies in the blood, serum or tissues of a mammal with cancer. Such antibodies can arise naturally within the mammal when the cancer-associated epitope becomes exposed during malignant transformation.

Accordingly, the invention provides a method of detecting cancer in a mammal by contacting a test sample with a cancer-associated epitope of the invention and detecting whether an antibody from the test sample has bound to the cancer-associated epitope.

Antibodies or binding entities that are reactive with cancer-associated epitope of the invention and/or polypeptides comprising a cancer-associated epitope of the invention can be labeled or coupled to a diagnostic imaging agent for convenient detection of cancer.

The words “label” and diagnostic imaging agent refer to a detectable compound or composition that is conjugated directly or indirectly to an antibody or antigen or epitope. The label may itself be detectable (e.g., radioisotope labels or fluorescent labels) or, in the case of an enzymatic label, may catalyze chemical alteration of a substrate compound or composition that is detectable.

Such labels or diagnostic imaging agents are useful for imaging of cells and tissues that express the cancer-associated epitope. Such labels can also be used with a cancer-associated epitope of the invention in standard immunoassays. Labels and diagnostic imaging agents include, but are not limited to barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium.

Paramagnetic isotopes for purposes of in vivo diagnosis can be used according to the methods of this invention. There are numerous examples of elements that are useful in magnetic resonance imaging. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415. Examples of suitable fluorescent labels include a fluorescein label, an isothiocyalate label, a rhodamine label, a phycoerythrin label, a phycocyanin label, an allophycocyanin label, an ophthaldehyde label, a fluorescamine label, etc. Examples of chemiluminescent labels include a luminal label, an isoluminal label, an aromatic acridinium ester label, an imidazole label, an acridinium salt label, an oxalate ester label, a luciferin label, a luciferase label, an aequorin label, etc. Those of ordinary skill in the art will know of other suitable labels that may be employed in accordance with the present invention.

The attachment of these labels to antibodies or fragments thereof can be accomplished using standard techniques commonly known to those of ordinary skill in the art. Typical techniques are described by Kennedy et al., (1976) Clin. Chim. Acta 70, 1-3 1; and Schurs et al., (1977) Clin. Chim. Acta 81, 1-40. Coupling techniques mentioned in the latter are the glutaraldehyde method, the periodate method, the dimaleimide method, the m-maleimidobenzyl-N-hydroxy-succinimide ester method. All of these methods are incorporated by reference herein.

A solid phase or a solid support can be used in conjunction with the antibodies, binding entities, antigens or epitopes of the invention. Such a solid phase or solid support refers to a non-aqueous matrix to which the antibody, binding entity, antigen or epitope can adhere. Examples of solid phases and supports encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase or support can comprise the well of an assay plate; in others it is a purification column (e.g. an affinity chromatography column). This term also includes a discontinuous solid phase of discrete particles, such as those described in U.S. Pat. No. 4,275,149.

Therapy

According to the invention, the antigenic epitopes of the invention, antibodies or binding entities directed against such epitopes and protease inhibitors that inhibit formation of the epitopes of the invention can be used for cancer prevention and/or therapy. The antigenic epitopes of the invention can be used as vaccines to stimulate an immunological response in a mammal that is directed against cells having the cancer-associated epitope. Antibodies or binding entities directed against the antigenic epitopes of the invention can combat or prevent adenocarcinomas. Moreover, the invention contemplates administering protease inhibitors that inhibit cleavage of cytokeratin 8 and/or cytokeratin 18 to prevent or treat adenocarcinomas.

In one embodiment, the invention provides a method of preventing or treating adenocarcinoma in a mammal by administering an antigenic epitope comprising any one of SEQ ID NO:3-6 to the mammal in an amount sufficient to stimulate an immunological response against the antigenic epitope. Two or more polypeptides comprising SEQ ID NO:3-6 can be combined in a therapeutic composition and administered in several doses over a period of time that optimizes the immunological response of the mammal. Such an immunological response can be detected and monitored by observing whether antibodies directed against the epitopes of the invention are present in the bloodstream of the mammal.

Antibodies and binding entities generated as provided herein that react selectively with the cancer-associated epitope of the invention also be used for cancer therapy. Accordingly, the invention provides methods of preventing or treating adenocarcinoma in a mammal by administering to the mammal a therapeutically effective amount of an antibody or binding entity that can bind an antigenic epitope comprising any one of SEQ ID NO:3-6.

Such antibodies or binding entities can be used alone or coupled to, or combined with, therapeutically useful agents. Antibodies and/or binding entities can be administered to mammals suffering from any cancer that displays the cancer-associated epitope of the invention. Such administration can provide both therapeutic treatment, and prophylactic or preventative measures. For example, the therapeutic methods of the invention can be used to deter the spread of a cancer and lead to its remission.

As used herein, “therapeutically useful agents” include any therapeutic molecule that can beneficially be targeted to a cell expressing the cancer epitope disclosed herein, including antineoplastic agents, radioiodinated compounds, toxins, chemotherapeutic agents, cytostatic or cytolytic drugs.

Such therapeutically useful agents include, for example, adrimycin, aminoglutethimide, aminopterin, azathioprine, bleomycin sulfate, bulsulfan, carboplatin, carminomycin, carmustine, chlorambucil, cisplatin, cyclophosphamide, cyclosporine, cytarabidine, cytosine arabinoside, cytoxin dacarbazine, dactinomycin, daunomycin, daunorubicin, doxorubicin, esperamicins (see U.S. Pat. No. 4,675,187), etoposide, fluorouracil, ifosfamide, interferon-α, lomustine, melphalan, mercaptopurine, methotrexate, mitomycin C, mitotane, mitoxantrone, procarbazine HCl, taxol, taxotere (docetaxel), teniposide, thioguanine, thiotepa, vinblastine sulfate, vincristine sulfate and vinorelbine. Additional agents include those disclosed in Chapter 52, Antineoplastic Agents (Paul Calabresi and Bruce A. Chabner), and the introduction thereto, pp.1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60, I-131, I-125, Y-90 and Re-186, and enzymatically active toxins of bacterial, fungal, plant or animal origin, or fragments thereof.

According to the invention, such chemotherapeutic agents can be used to reduce the growth or spread of cancer cells and tumors that express the tumor associated epitope of the invention. Animals that can be treated by the chemotherapeutic agents of the invention include humans, non-human primates, cows, horses, pigs, sheep, goats, dogs, cats, rodents and the like. In all embodiments human tumor antigens and human subjects are preferred.

The invention also contemplates using species-dependent antibodies for use in the present therapeutic methods. Such a species-dependent antibody has constant regions that are substantially non-immunologically reactive with the chosen species. Such species-dependent antibody is particularly useful for therapy because it gives rise to substantially no immunological reactions. The species-dependent antibody can be of any of the various types of antibodies as defined above, but preferably is mammalian, and more preferably is a humanized or human antibody.

Therapeutically useful agents can be formulated into a composition with the antibodies of the invention and need not be directly attached to the antibodies of the invention. However, in some embodiments, therapeutically useful agents are attached to the antibodies of the invention using methods available to one of skill in the art, for example, standard coupling procedures.

The invention further provides methods of preventing or treating adenocarcinoma in a mammal by administering to the mammal a therapeutically effective amount of a protease inhibitor that prevents formation of an antigenic epitope comprising any one of SEQ ID NO:3-6. According to the invention, the sites of protease cleavage at amino acids 22 and 40 on cytokeratin K8, and at amino acid 50 on cytokeratin K18, all contained consensus sequence Xaa₁SR↓Xaa₄ (SEQ ID NO:40), where Xaa₁ is serine, phenylalanine or valine and Xaa₄ is serine or valine. The structure of these cleavage sites indicates that the enzyme responsible for these cleavages is a trypsin-like protease. Trypsin inhibitors are available to one of skill in the art. See, e.g., U.S. Pat. No. 6,239,106; U.S. Pat. No. 6,159,938; U.S. Pat. No. 5,962,266. Such trypsin inhibitors include inhibitors available for serine proteases such as kallikrein, chymotrypsins A and B, trypsin, elastase, subtilisin, coagulants and procoagulants, particularly those in active form, including coagulation factors such as factors VIIa, IXa, Xa, XIa, and XIIa, plasmin, thrombin; proteinase-3, enterokinase, acrosin, cathepsin, urokinase, and tissue plasminogen activator.

According to the invention, any inhibitor capable of inhibiting a protease that can cleave Xaa₁SR↓Xaa₄ (SEQ ID NO:40) may be used to prevent or treat adenocarcinomas. For example, peptides with homology to Xaa₁SR↓Xaa₄ (SEQ ID NO:40) but that cannot be cleaved may be used as inhibitors in the present therapeutic methods. Other examples of inhibitors that may be used include, for example, soybean trypsin inhibitor (or STI, from Sigma Chemical Co.), alpha-2-macroglobulin, alpha-1-antitrypsin, aprotinin, pancreatic secretory trypsin inhibitor (PSTI) corn and pumpkin trypsin inhibitors (Wen, et al., Protein Exp. & Purif. 4:215 (1993); Pedersen, et al., J. Mol. Biol. 236:385 (1994)), and so forth. One candidate for a useful inhibitor of human origin is found in circulating isoforms of the human amyloid β-protein precursor (APPI), also known as protease nexin-2. APPI contains a Kunitz serine protease inhibitor domain known as KPI (Kunitz Protease Inhibitor). See Ponte et al., Nature, 331:525 (1988); Tanzi et al., Nature 331:528 (1988); Johnstone et al., Biochem. Biophys. Res. Commun. 163:1248 (1989); Oltersdorf et al., Nature 341:144 (1989). Human KPI shares about 45% amino acid sequence identity with aprotinin. The isolated KPI domain has been prepared by recombinant expression in a variety of systems, and has been shown to be an active serine protease inhibitor. See, for example, Sinha, et al., J. Biol. Chem. 265:8983 (1990).

Progression of adenocarcinoma cancer and/or the therapeutic efficacy of chemotherapy may be measured using procedures available in the art. For example, the efficacy of a particular chemotherapeutic agent can be determined by measuring the amount of cancer-associated epitope released from adenocarcinoma cells undergoing cell death. The concentration of antigenic epitope (e.g. a polypeptide having any one of SEQ ID NO:3-6, or a combination of such polypeptides) released from cells can be compared to standards from healthy, untreated patients to assess whether heightened levels of the present epitopes are present in a patient. Fluid samples can be collected at discrete intervals during treatment and compared to a standard. It is contemplated that changes in the level of a cancer-associated antigenic epitope of the invention, will be indicative of the efficacy of treatment (that is, the rate of cancer cell death). It is contemplated that the release of cancer-associated antigenic epitopes can be measured in many test samples, including blood, plasma, serum, feces, urine, sputum, vaginal secretions, seminal fluids, semen and any tissue sample.

Where the assay is used to monitor tissue viability or progression of adenocarcinoma, the step of detecting the presence and abundance of the antigenic epitope in samples of interest is repeated at intervals and these values then are compared, the changes in the detected concentrations reflecting changes in the status of the tissue. For example, an increase in the level of adenocarcinoma-associated epitope may correlate with progression of the adenocarcinoma. Where the assay is used to evaluate the efficacy of a therapy, the monitoring steps occur following administration of the therapeutic agent or procedure (e.g., following administration of a chemotherapeutic agent or following radiation treatment). Similarly, a decrease in the level of adenocarcinoma cancer-associated epitopes of the invention may correlate a regression of the adenocarcinoma.

Thus, adenocarcinomas may be identified by the presence of cancer-associated antigenic epitopes as provided herein. Once identified, the adenocarcinoma may be treated using antibodies and protease inhibitors that reduce cleavage of cytokeratins 8 and 18. Moreover, the methods provided herein can be used to monitor the progression of the disease and/or treatment of the disease.

Compositions

The invention is further directed to compositions containing the present antibodies, binding entities, antigenic epitopes or trypsin-like protease inhibitors. Such compositions are useful for detecting the antigenic epitopes of the invention and for therapeutic methods involving prevention and treatment of cancers associated with the presence of the antigenic epitopes of the invention.

The antibodies, binding entities, antigenic epitopes and protease inhibitors of the invention can be formulated as pharmaceutical compositions and administered to a mammalian host, such as a human patient in a variety of forms adapted to the chosen route of administration. Routes for administration include, for example, intravenous, intra-arterial, subcutaneous, intramuscular, intraperitoneal and other routes selected by one of skill in the art.

Solutions of the antibodies, binding entities, antigenic epitopes and protease inhibitors of the invention can be prepared in water or saline, and optionally mixed with a nontoxic surfactant. Formulations for intravenous or intra-arterial administration may include sterile aqueous solutions that may also contain buffers, liposomes, diluents and other suitable additives.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating the antibodies, binding entities, antigenic epitopes and protease inhibitors in the required amount in the appropriate solvent with various of the other ingredients enumerated above, as required, followed by filter sterilization.

Useful dosages of the antibodies, binding entities, antigenic epitopes and protease inhibitors can be determined by observing their in vitro activity, and in vivo activity in animal models. Methods for the extrapolation of effective dosages in mice, and other animals, to humans are known to the art; for example, see U.S. Pat. No. 4,938,949.

In general, a suitable dose of the antibodies, binding entities, antigenic epitopes and protease inhibitors will be in the range of from about 1 to about 2000 μg/kg, for example, from about 2.0 to about 1500 μg/kg of body weight per treatment. Preferred doses are in the range of about 3 to about 500 μg per kilogram body weight of the recipient per treatment, more preferably in the range of about 10 to about 300 μg/kg/treatment, most preferably in the range of about 20 to about 200 μg/kg/treatment.

The antibodies, binding entities, antigenic epitopes and protease inhibitors are conveniently administered in unit dosage form; for example, containing 5 to 1000 μg, conveniently 10 to 750 μg, most conveniently, 50 to 500 μg of active ingredient per unit dosage form.

Ideally, the antibodies, binding entities, antigenic epitopes and protease inhibitors should be administered to achieve peak plasma concentrations of from about 0.1 to about 10 nM, preferably, about 0.2 to 10 nM, most preferably, about 0.5 to about 5 nM. This may be achieved, for example, by the intravenous injection of a 0.05 to 25% solution of the antibodies, optionally in saline. Desirable blood levels may be maintained by continuous infusion to provide about 0.01-10.0 μg/kg/hr or by intermittent infusions containing about 0.4-50 μg/kg of the antibodies.

The desired dose may conveniently be presented in a single dose or as divided doses administered at appropriate intervals, for example, as two, three, four or more sub-doses per day. The sub-dose itself may be further divided, for example, into a number of discrete loosely spaced administrations; such as multiple intravenous doses. For example, it is desirable to administer the present compositions intravenously over an extended period, either by continuous infusion or in separate doses.

Kits

The invention further provides kits for detection of the antigenic epitope of the invention and for treatment of adenocarcinomas.

A kit for detection of the antigenic epitope of the invention may contain a container containing an antibody or binding entity capable of binding to an antigenic epitope of the invention. Such an antibody or binding entity may be labeled for easy detection. Individual kits may be adapted for performing one or more of the methods of the invention.

Optionally, the subject kit may further comprise at least one other reagent required for performing the method that the kit is adapted to perform. Examples of such additional reagents include: a label, a standard, a control, a buffer, a solution for diluting the test sample, or a reagent that facilitates detection of the label. The reagents included in the kits of the invention may be supplied in premeasured units so as to provide for greater precision and accuracy. Typically, kits reagents and other components are placed and contained in separate vessels. A reaction vessel, test tube, microwell tray, microtiter dish or other container can also be included in the kit. Different labels can be used on different reagents so that each reagent can be distinguished from another.

A further aspect of the invention relates to a kit for treatment of adenocarcinomas comprising a pharmaceutical composition of the invention and an instructional material. Such a kit may contain a container having an antigenic epitope, an antibody, a binding entity or an inhibitor of the invention. The antigenic epitope may act as a vaccine for preventing formation of metastatic adenocarcinoma. The antibody or binding entity is directed against an antigenic epitope of the invention and can be administered to treat or prevent the spread of adenocarcinomas. An inhibitor of cytokeratin 8 or 18 cleavage can also inhibit the formation and spread of adenocarcinomas. Any one of these antigenic epitopes, antibodies, binding entities or inhibitors may be contained within an appropriate container in the kit. Alternatively, a combination of antigenic epitopes, antibodies, binding entities or inhibitors may be contained within an appropriate container in the kit.

As used herein, an “instructional material” includes a publication, a recording, a diagram, or any other medium of expression that is used to communicate the usefulness of the pharmaceutical composition of the invention for inhibiting cleavage of cytokeratin 8 or 18 or for stimulating the immune system to recognize the epitopes of the invention in a mammal or patient. The instructional material may also, for example, describe an appropriate dose of the pharmaceutical composition of the invention. The instructional material of the kit of the invention may, for example, be affixed to a container that contains a pharmaceutical composition of the invention or be shipped together with a container that contains the pharmaceutical composition. Alternatively, the instructional material may be shipped separately from the container with the intention that the instructional material and the pharmaceutical composition be used cooperatively by the recipient.

The invention also includes a kit comprising a pharmaceutical composition of the invention and a delivery device for delivering the composition to a mammal, for example, a human patient who may have an adenocarcinoma. By way of example, the delivery device may be a squeezable spray bottle, a metered-dose spray bottle, an aerosol spray device, an atomizer, a dry powder delivery device, a self-propelling solvent/powder-dispensing device, a syringe, a needle, a tampon, or a dosage measuring container.

The invention will be further described by reference to the following detailed examples, which are given for illustration of the invention, and are not intended to be limiting thereof.

### EXAMPLE 1

### Cancer-Associated Epitope Characterization Isolation of COU-1 Monoclonal Antibodies

The IgM HMab, COU-1, is secreted by the hybridoma cell line, B9165, derived by fusing the human lymphoblastoid cell line WI-L2-729-HF2 with lymphocytes obtained from mesenteric lymph nodes from a colon cancer patient (35). Mesenteric lymph nodes draining the tumor region in patients with colorectal cancer were minced under sterile conditions. Debris was removed by filtration through cotton wool and the lymphocytes were purified by centrifugation on Ficoll-Isopaque (Boehringer-Mannheim, Mannheim, Federal Republic of Germany).

The lymphocytes were fused with the human fusion cell line WI -L2-729-HF2 (referred to as HF2) (from Tecniclone Int., Santa Ana, Calif., USA) according to Kohler, Immunological Methods Vol. II, Academic Press, 1981, pp. 285-298. The ratio between the HF2 and lymphocytes (10⁷) was 1:2.

After washing the HF2 and the lymphocytes together in RPMI-1640 medium and collecting the cells by centrifugation, the cell pellet was resuspended in 0.5 ml of 50% polyethylene glycol (PEG) 6000 over a period of 1 minute with constant shaking. Before dilution of the PEG with RPMI-1640, the cells were incubated for another 2 minutes. The resulting fusion product was washed and resuspended in solution medium [RPMI-1640, 10% FCS (fetal calf serum) supplemented with HAT (2×10⁻⁴ M hypoxanthine, 4×10⁻⁷ M aminopterin, 3.2×10⁻⁶ M thymidine)]. The cells were plated in 96-well microtiter plates using 200 μl containing 2×10⁵ cells per well. The cells were maintained in selective medium for two weeks. Further culturing was carried out in RPNI-1640 with 10% FCS supplemented with hypoxanthine and thymidine. Growing hybrids appeared 10 days to 4 weeks after fusion. Cloning was performed by limiting dilution without feeder cells.

Supernatants from wells with growing clones were analyzed for immunoglobulin production by ELISA using microtiter plates coated with rabbit anti-human Ig (H and L chain) (Dakopatts, Copenhagen, Denmark) diluted 1:10,000 in 0.1M bicarbonate, pH 9.6. Coated wells were washed with PBS-Tween (phosphate buffered saline-0.05% Tween 20) and incubated for 2 hours at room temperature with supernatants diluted 1:10 in PBS-Tween. Development was carried out with alkaline phosphatase (AP)-coupled antibody specific for IgM, IgA or IgG (Dakopatts, Copenhagen) diluted 1:3000 in PBS-Tween. After incubation for 1 hour at room temperature, the substrate p-nitrophenylphosphate (PNPP), 1 mg/ml 10% diethanolamine, 1 mM MgCl2, pH 9.6, was added. Optical density was measured at 405 nm after 1 hour of incubation at 37° C. Standard curves for quantification were constructed with dilution of IgM (Cappel) or IgG (Kabi AB, Stockholm, Sweden). Hybrids producing immunoglobulin (Ig) assayed by ELISA were propagated by transfer to 24-well macroplates (Nunc A/S, Denmark). The hybridoma cell line B9165 (ECACC 87040201) selected by the methods secreted the COU-1 antibodies described below and was shown by ELISA to produce between 1 and 5 μg of IgM per ml when allowed to grow for two weeks without change of media.

The hybridoma cell line B9165 was deposited with European Collection of Cell Cultures (ECACC), CAMR, Salisbury, Wiltshire, SP4 OJG, UK, Deposit no. ECACC 87040201.

COU-1 hybridoma supernatants were further analyzed by immunocytochemical analysis for reaction with tumor cells or by immunohistochemical analysis for reactions with tumor tissues as described below.

Immunocytochemical Analysis of COU-1 Antibodies

Immtinocytochemical analysis was performed on cell smears prepared from different human tumor cell lines and from peripheral human blood leukocytes. Cells were fixed on slides by treatment with formol-acetone (9.5% formaldehyde, 43% acetone in 86 mM phosphate buffer, pH 7.2). Approximately 50 μl of COU-1 supernatant (from the hybridoma B9165; ECACC 87040201) was placed on the smear of fixed cells and incubated overnight at 4° C. in a humidified chamber before rinsing and incubation for 1 hour at room temperature with horseradish peroxidase (HRP)-labeled rabbit anti-human IgM (Dakopatts) diluted to 1:80 in PBS-Tween. Finally, peroxidase substrate (0.01% H₂O₂ and diaminobenzidine at 0.6 μg/ml in PBS) was added. The smears were lightly counterstained with hematoxylin and mounted. Table 3 shows the results obtained by analysis of COU-1 on smears of various cells.

TABLE 3
Reactivity of COU-1 assayed by immunocytochemistry

| Type of cell | Name | Reaction |
|---|---|---|
| 1. Colon adenocarcinoma | Colon 137 | Positive |
| 2. Colon adenocarcinoma | COLO 201 | Positive |
| 3. Melanoma | HU 373 | Negative |
| 4. Mammary carcinoma | MCF-7 | Positive |
| 5. Duodenal adenocarcinoma | HUTU 80 | Negative |
| 6. Burkitt's lymphoma | EB-2 | Negative |
| 7. Human peripheral blood | PBL | Negative |
| Leukocytes | | |

A selective reactivity with colon and mammary adenocarcinomas was apparent.

Live COLO 201 cells (colonic adenocarcinoma cells) were incubated with the COU-1 antibody at 4° C., followed by the enzyme-labeled anti-Ig antibody. The cells were then smeared on slides, fixed with glutaraldehyde (0.17% in PBS) and incubated with substrate. COLO 201 cells stained with COU-1 while control cells did not (data not shown).

Immunohistochemical Analysis.

A preliminary immunohistochemical analysis was performed on frozen tissue sections fixed in acetone. Endogenous IgM was blocked by incubation with Fab′ fragments of anti-μ-chain antibody (purchased from Dakopatts, Copenhagen, Denmark) before incubation with the COU-1 antibody (0.5 μg/ml). The anti-mu-chain antibody Fab′ fragment was prepared according to B. Nielsen et al., Hybridoma 6 (1), 1987, pp. 103-109). While clear-cut specificity for cancerous tissues was observed using the COU-1 antibody, some non-specific binding was observed in certain tissue types (for example, mammary tubules).

An improved fixation procedure was used that substantially eliminated non-specific cross-reactivity with certain tissue types, including mammary ductuli and tubules. Tissue specimens were obtained from colorectal cancer patients undergoing surgical resection. Normal colon tissue was taken from the resectate approximately 15 cm away from the site of the tumor. Tissues were fixed in 96% alcohol for 6 h at 4° C. Afterwards, tissues were paraffin embedded and cut into 5 μm sections. Sections were deparaffinized in xylol, rehydrated through graded alcohol and washed in PBS-Tween. Sections were incubated for 2 h at room temperature in a humidified chamber with 100 μl of murine monoclonal antibody, human monoclonal IgM antibody or normal polyclonal human IgM, all at 0.5-10 μg/ml. The slides were washed and incubated with AP-labeled rabbit anti-human IgM (Dako, Glostrup, Denmark), horse-radish peroxidase (HRP) labeled rabbit anti-human IgM (Dako) or HRP-labeled rabbit anti-mouse IgG (Dako) diluted in PBS with 10% (w/v) bovine serum albumin for 1 h at room temperature. After washing, the HRP was visualized by development with chromogenic substrate (0.6 mg diaminobenzidine per ml PBS with 0.01% H₂O₂) and AP with 0.2 mg naphthol-AS-Mx phosphate (Sigma), 1 mg Fast Red TR Salt (Sigma), 20 μg dimethylformamide per ml 0.1M Tris/HCl, 1M levamisole, pH 8.2. The sections were counterstained with Mayer's haematoxylin, dehydrated in xylene and mounted in Aquamount (Gurr, Poole, England).

Bound antibody was visualized as described above for the immunocytochemical analysis. Only the tumor cells in sections of colon adenocarcinomas were stained COU-1. No staining was observed in tonsillar tissue. Tables 4A and 4B summarize the reactivity of the COU-1 antibody with a variety of tissues, where the reactivity of malignant tissues is provided in Table 4A and the lack of reactivity of non-malignant tissues is provided in Table 4B.

TABLE 4A
COU-1 Antibody Reactivity vs. Malignant Human Tissues

| Tissue | Result^{a} |
|---|---|
| Colon adenocarcinoma | 19/21 |
| Ovarian adenocarcinoma | 2/2 |
| Renal adenocarcinoma | 1/2 |
| Mammary carcinoma | 7/9 |
| Lung adenocarcinoma | 7/7 |
| Non-seminomal testis carcinoma | 1/1 |
| Sarcoma | 0/3 |
| Malignant melanoma | 0/7 |
| B-lymphoma | 0/1 |
| Thymoma | 0/1 |

^{a}Number positive/Number tested.

TABLE 4B
COU-1 Antibody Reactivity vs. Normal Human Tissues

| Tissue | Result |
|---|---|
| Ovarian stroma | Negative |
| Ovarian epithelia | Negative |
| Renal glomeruli | Negative |
| Renal tubules | Negative |
| Lung alveoles | Negative |
| Bronchial epithelium | Negative |
| Testis | Negative |
| Epidermis | Negative |
| Tonsillary lymphatic tissue | Negative |
| Tonsillary epithelium | Negative |
| Smooth muscles | Negative |
| Blood vessels | Negative |
| Prostate epithelium | Positive |

Normal colon epithelium showed binding of all analyzed human IgM, monoclonal antibodies, myeloma IgM as well as normal polyclonal human IgM. This general binding of IgM to normal colon epithelium was thus judged to be non-specific.

### EXAMPLE 2

### Cancer-Associated Epitope Mapping Materials and Methods

Antibodies

The IgM HMab, COU-1, is secreted by the hybridoma cell line, B9165, derived by fusing the human lymphoblastoid cell line WI-L2-729-HF2 with lymphocytes obtained from mesenteric lymph nodes from a colon cancer patient, as described above. The hybridoma cell line B9165 was deposited with European Collection of Cell Cultures (ECACC), CAMR, Salisbury, Wiltshire, SP4 OJG, UK, Deposit no. ECACC 87040201. More information about ECACC can be obtained on the website at ecacc.org.

The human-human hybridoma cell line was grown in protein-free medium: RPMI 1640 medium (Gibco, Grand Island, N.Y.) supplemented with SSR3 serum replacement (Medicult, Copenhagen, Denmark). HMab COU-1 was purified from cell culture supernatant by affinity chromatography on Sepharose-coupled murine anti-human μ-chain monoclonal antibody (Mab)(HB57, ATCC, Rockville, Md.). The antibody was eluted with 0.1 M diethylamine, pH 10.5, followed by fractionation by FPLC. IgM purified from normal human serum (Cappel, Cochranville, Pa.) was used as a control. Murine Mabs, M20 directed against normal K8 and CY-90 directed against normal K18, were obtained from Sigma Chemical Co. (St. Louis, Mo.).

ELISA

ELISA wells (Costar, Cambridge, Mass.) were coated overnight at 4° C. with fractions from cytokeratin purification procedures or with different recombinant K8/K18 complexes (5 μg/ml) in PBS, pH 7.4. The wells were washed twice with PBS, blocked with 3% BSA in PBS for 1 h at 37° C., and incubated with HMab COU-1 antibody for 2 h at 37° C. Plates were washed 10× with PBS-0.05% Tween 20 and bound antibody was detected with alkaline phosphatase (AP)-labeled goat anti-human kappa-chain (Sigma) diluted 1000 fold in PBS. Bound antibody was visualized with para-nitrophenylphosphate (Sigma)(1 mg/ml 1 mM MgCl₂, 10% (w/v) diethanolamine, pH 9.6) and read at 405 nm.

Cell Culture

The human breast adenocarcinoma cell line MCF7 (ATCC) was maintained in Eagle's MEM (Gibco), supplemented with 10% FCS, non-essential amino acids, 1 mM sodium pyruvate, 1 mM HEPES buffer, 100 U penicillin/ml, 100 mg streptomycin/ml and 2 mM L-glutamine. The human colon adenocarcinoma cell line Colon 137 (kindly provided by Dr. Ebbesen, Aarhus University, Denmark) was maintained in RPMI 1640 (Gibco), supplemented with FCS, penicillin, streptomycin and L-glutamine as above.

Purification of Cytokeratin from Normal and Malignant Tissue

Cytokeratin were prepared from fresh, surgically-removed, colon cancer tissue or normal colon epithelia. Tissue samples (1-5 g) were minced with a shears and homogenized in 10-30 ml of Tris-buffered saline (TBS)(10 mM Tris, 0.14 M NaCl, 15 mM NaN₃.pH 7.6) containing 1% (v/v) Emulphogene (Sigma) using a blade rotor (Euro Turrax T20b basic, IKA Labortechnik, Staufen, Germany) for 3×5 sec at 27.000 rpm on ice. Enzyme inhibitors: 5 mM iodoacetamide, 10 mM PMSF, 5 mM EDTA (all Sigma), 5 mM Cyclocapron (KABI, Stockholm, Sweden), and 10 U Aprotinin (Bayer, Leverkusen, Germany) per ml were included in the buffers during the homogenization, sonication and ion exchange chromatography. The suspension was pelleted by centrifugation at 10.000 g for 10 min at 4° C., washed twice in TBS containing 1% Emulphogene and resuspended in buffer A (10 mM Tris pH 8.6 containing 0.1% SDS (w/v) and 0.05% Emulphogene). The suspension was sonicated for 3×15 sec on ice and centrifuged at 12.000 g for 10 min at 4° C. The supernatant was applied to an anion exchange column (20 ml Q-Sepharose Fast Flow column, QFF, (Pharmacia Upjohn, Uppsala, Sweden)) pre-equilibrated with buffer A. After washing the column with 10 column volumes of buffer A, bound proteins were eluted with a linear gradient to 1M NaCl in buffer A. Fractions of 1 ml were collected and further analyzed by SDS-PAGE/Western blotting and ELISA. For ELISA, 10 μl of each fraction was added to wells containing 10 μl of SM2 beads (BioRad) in 100 μl TBS, followed by incubation with COU-1 as described above. The beads bind the detergent and thus allow for the direct coating of the proteins in the fractions.

SDS-PAGE and Western Blot Analysis

Electrophoresis was performed in a discontinuous buffer system on 8 cm 4-20% or 10% (w/v) polyacrylamide gels for analysis and on 15 cm 14% polyacrylamide gels for N-terminal sequencing (36). Samples were mixed with 2× sample buffer (4% SDS, 0.2% bromophenol blue, 20% glycerol in 100 mM Tris buffered saline), boiled for 5 min and resolved under denaturing and reducing (100 mM DTT) conditions. Protein bands were visualized with Coomassie Brilliant Blue. Separated proteins were also electroblotted onto polyvinylidene difluoride membranes (PVDF, Immobilon P, Millipore, Bedford, Mass.), at 100 Volts for 1 h in ice, using transfer buffer (10% (v/v) ethanol, 25 mM Tris, 200 mM glycine). Prior to transfer, the membrane was pre-soaked in ethanol for 2 min and the membranes and the gel were incubated in transfer buffer for 10 min. Following transfer, the membrane was blocked for 2 h in Western blot buffer (50 mM Tris, 350 mM NaCl, 15 mM NaN₃, 0.1% Tween-20) washed 3× with Western blot buffer and incubated with COU-1 antibody (5 μg/ml), mouse anti-K8 antibody (diluted 1/2000), mouse anti K-18 antibody (diluted 1/2000) or goat-anti-GST antibody (diluted 1/1000, Pharmacia Upjohn) overnight at room temperature. The membrane was washed in Western blot buffer and incubated with AP-conjugated rabbit-anti-goat IgG antibody (diluted 1/1000, Sigma), or AP-conjugated rabbit-anti-human IgM antibody (diluted 1/500, DAKO, Glostrup, Denmark) for 2 h at room temperature. Following 3 washes in PBS, the membrane was fixed with 0.2% glutaraldehyde in PBS for 15 min at room temperature and finally washed in PBS. Bound AP conjugate was visualized by NBT/BCIP (Bio-Rad, Hercules, Calif.). MCF7 or Colon 137 cells, resuspended in SDS sample buffer and sonicated, were used as antigen control. A low range protein marker (Bio-Rad) was used to indicate the molecular weight of the fragments.

Amino Acid Sequencing and Amino Acid Analysis

Previously described procedures (37) were employed for amino acid sequencing and amino acid analysis. For N-terminal sequencing, purified cytokeratin was run on SDS-PAGE and electroblotted onto PVDF membranes prior to detection with Coomassie. The different bands were excised from the blot and sequenced in an Applied Biosystems 470A protein sequencer (ABI, Forster City, Calif.). Sequences similar to cytokeratins were searched for in GenBank/EBI/DDBI/PDB databases using the BLAST program.

Expression and Purification of Recombinant K8 and K18 Proteins

E. coli DH5a harboring plasmids encoding a panel of K8 and K18 proteins were analyzed. The panel consisted of the full length and several N-terminal and C-terminal deleted fragments of K8 and K18, cloned as GST fusion proteins into a modified pGEX-2T vector (38). The E. coli cultures were grown in Super Broth medium, supplemented with 20 mM MgCl₂ and 50 mg carbenicillin/ml at 37° C. until OD₆₀₀ reached 0.6. Protein expression was then induced with 1 mM IPTG (Sigma) and 4 μM cAMP and the culture allowed to grow for an additional 3 h at 30° C. The bacteria were pelleted at 4.000 g for 15 min at 4° C. For SDS-PAGE, the pellet was resuspended in sample buffer and sonicated 5×10 sec before electrophoresis. For purification of the recombinant K8 or K18 proteins, the pellet of a 400 ml culture grown and processed as described above was resuspended in 50 ml lysis buffer (50 mM Tris-HCl, 100 mM NaCl, 1 mM EDTA, 5 mM β-mercaptoethanol, pH 8.0) containing 1 mg/ml lysozyme and incubated for 30 min at 4° C. The suspension was sonicated 3×20 sec and pelleted at 20.000 g at 4° C. The pellet was washed twice in a high salt buffer (50 mM Tris-HCl, 2 M NaCl, 10 mM EDTA, 5 mM β-mercaptoethanol, 1% NP40, pH 8.0) and once in lysis buffer. The pellet was subsequently washed twice in lysis buffer containing 2 M urea and stored at 4° C. in lysis buffer containing 8 M urea.

Heterotypic Association Assay

Panels of different C- or N-terminal-deleted or intact K8 and K18 proteins were separated by SDS-PAGE and transferred to a PVDF membrane, as described above. After blocking, the membrane was incubated for 16 h at 4° C. with 100 μg/ml of purified K8 or K18 protein in PBS, 2% BSA and 4 M urea; if K8 proteins were transferred to the membrane, the membrane was subsequently incubated with a purified K18 protein, and vice versa (38). The membrane was then washed with PBS, incubated with COU-1 (5 μg/ml) in Western blot buffer containing 10% FCS for 2 h at room temperature and binding detected as described above.

Surface Plasmon Resonance

The kinetics of HMab COU-1 binding to heterotypic complexes of recombinant intact K8 or K18 (and fragments thereof) was determined by surface plasmon resonance measurements using the BIAcore instrument (Pharmacia). The sensor chip was activated for immobilization with N-hydroxysuccinimide and N-ethyl-N′-(3-diethyl aminopropyl) carbodiimide. The heterotypic cytokeratin complexes were coupled to the surface by injection of 50 μl of a 50 μg/ml sample. Excess activated esters were quenched with 30 μl 1 M ethanolamine, pH 8.5. Typically, 3000 resonance units were immobilized. Binding of COU-1 to immobilized heterotypic cytokeratin complexes was studied by injecting COU-1 in a range of concentrations (0.5-80 μg/ml) at a flow rate of 5 μl/min. The association was monitored as the increase in resonance units per unit time. Dissociation measurements were obtained following the end of the association phase with a flow rate of 20 μl/min. The binding surface was regenerated with 10 mM HCl, 1M NaCl, pH 2.0, and remained active for 10 measurements. The association and dissociation rate constants, kₒₙ and k_{off}, were determined from a series of measurements, as described previously (39). Association and dissociation constants were deduced from kinetic data using the Bioevaluation program version 3.1 (Pharmacia).

Confocal Laser Scanning Microscopy

Cells were seeded into Lab Tek chamber slides (Nalge Nunc, Naperville, Ill.) and allowed to grow and adhere to the glass slides for 48 h at 37° C., 5% CO₂. Cells were fixed with ice-cold 96% ethanol for 5 min, washed 3× with PBS and blocked with 10% normal goat serum in PBS for 1 h at room temperature. COU-1 (5 μg/ml) together with either mouse anti-K8 antibody (1/1000) or mouse anti K-18 antibody (1/1000) were incubated overnight at 4 ° C. After washing with PBS, the cells were incubated with FITC-labeled goat-anti-human γ-chain and Texas Red-labeled goat anti-mouse IgG antibody (diluted 1/200, both from Jackson ImmunoResearch, West Grove, Pa.) for 1 h at room temperature in the dark. The cells were washed with PBS for 3×5 min and the slides mounted with anti-fading reagent Slow Fade™ in PBS/glycerol (Molecular Probes, Eugene, Oreg.). Results were analyzed using a MRC-1024 confocal laser scanning microscope (Bio-Rad), attached to a Zeiss Anyvert 100TV. As a control, all experiments were also performed omitting the primary antibody or including species and isotype matched control antibody instead of the primary antibody. In addition, differential interference contrast (DIC) images of analyzed cells were obtained.

Results

Purification of Cytokeratins from Colon Cancer and Normal Colon Epithelia

Fresh, surgically-removed colon cancer tissue and normal colon epithelia was used to separately extract cytokeratin K8 an K18 by taking advantage of the fact that cytokeratins and other cytoskeletal proteins are present as insoluble filamentous structures in buffer at physiological salt concentrations. Non-ionic detergent was added to the buffer to improve homogenization, partially by disrupting cell membranes. The insoluble intermediate filaments proteins were precipitated by centrifugation, subsequently solubilized in an SDS-containing buffer, and separated by QFF anion-exchange chromatography using a linear salt gradient.

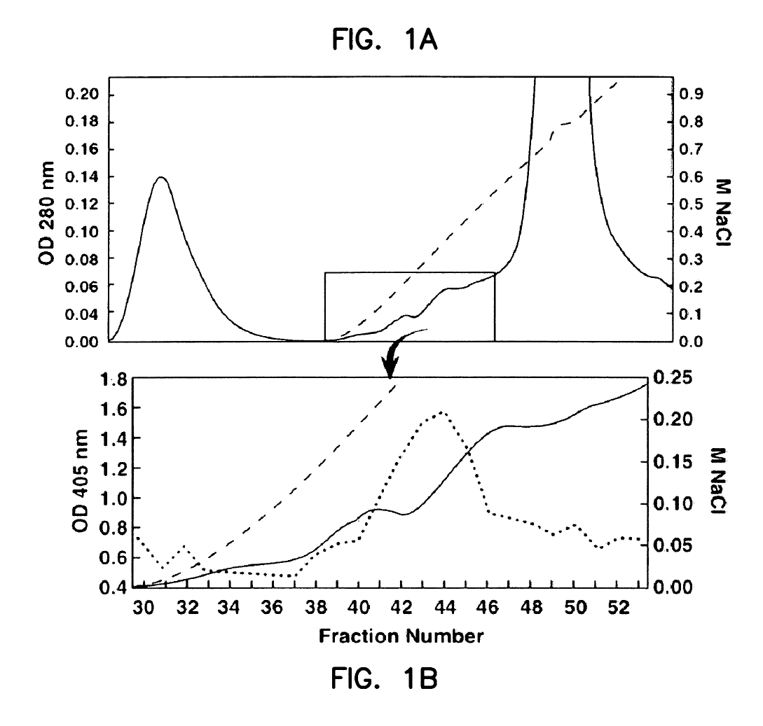

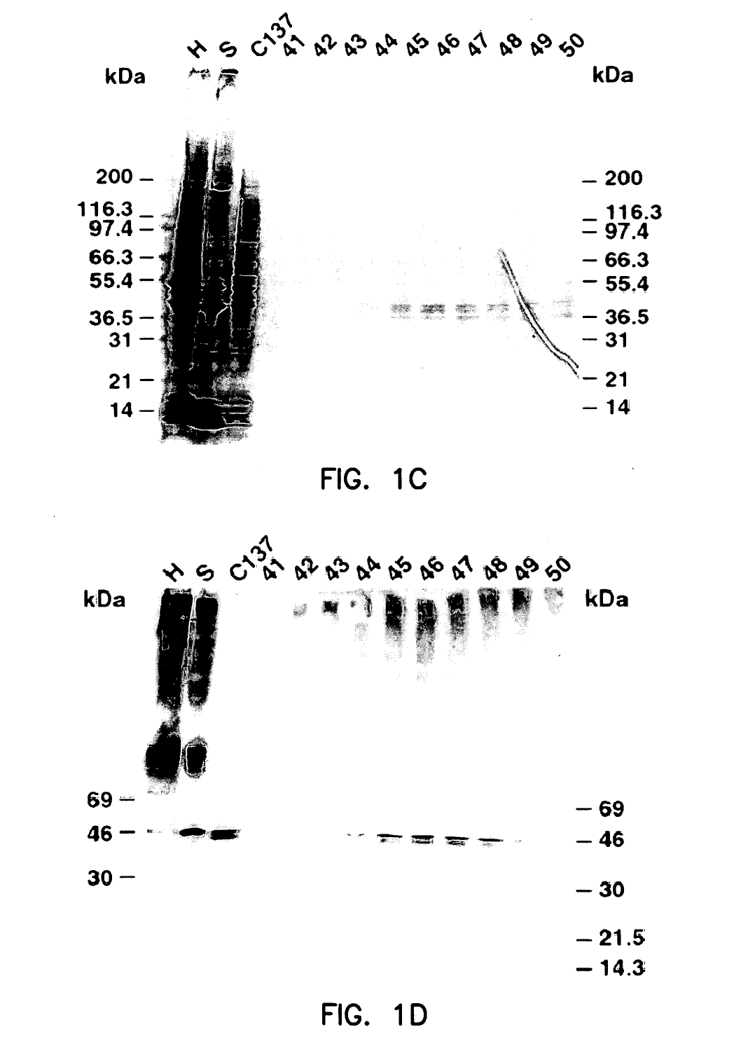

FIG. 1A shows the elution profile from the QFF anion-exchange column. The fractions containing COU-1 reactivity were found in the first and second peak of the gradient (fractions 41-48). COU-1 reactivity was detected by coating the proteins in these fractions onto ELISA wells followed by incubation with COU-1 (FIG. 1B). Western blot analysis demonstrated reactivity of COU-1 with three main bands in the same fractions (FIG. 1D). The proteins in these three bands represented only a portion of the proteins with molecular weight in the 41-46 kDa range found in these fractions, as revealed by Coomassie staining of the SDS-separated gels (FIG. 1C).

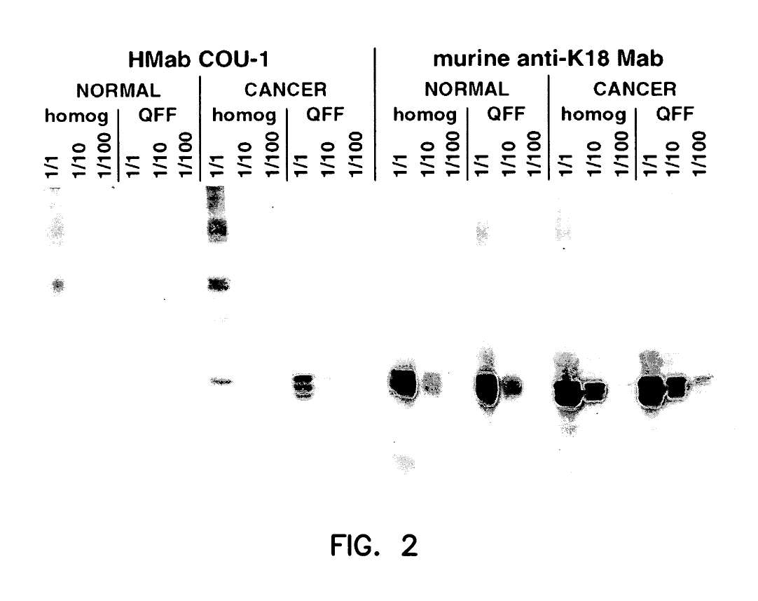

Western blot analysis and Coomassie staining of cytokeratin purified from colon cancers of four different patients revealed a similar pattern of protein bands, reactive and non-reactive with HMab COU-1. Cytokeratin was also isolated from normal colon epithelia obtained from three individuals using the same purification procedure to compare the nature of the K8/K18 in colon cancer versus normal colon tissues. Tissue homogenate and purified cytokeratin preparations (QFF eluate) from the two sources were examined by Western blotting using a panel of anti-K8 and anti-K18 antibodies. When approximately equal amounts of cytokeratin from cancer and the normal epithelia were analyzed, protein bands (in the 42-46 kDa range) of equal intensity were observed following staining with the anti-K18 antibody, CYK-90, which recognizes a linear epitope in the C-terminal part of K18 (FIG. 2). In contrast, when the same preparations were stained with COU-1, three bands at molecular weights of 42-46 kDa were stained in the cytokeratin preparations from the colon cancer tissue but not from the normal colon epithelia (FIG. 2).

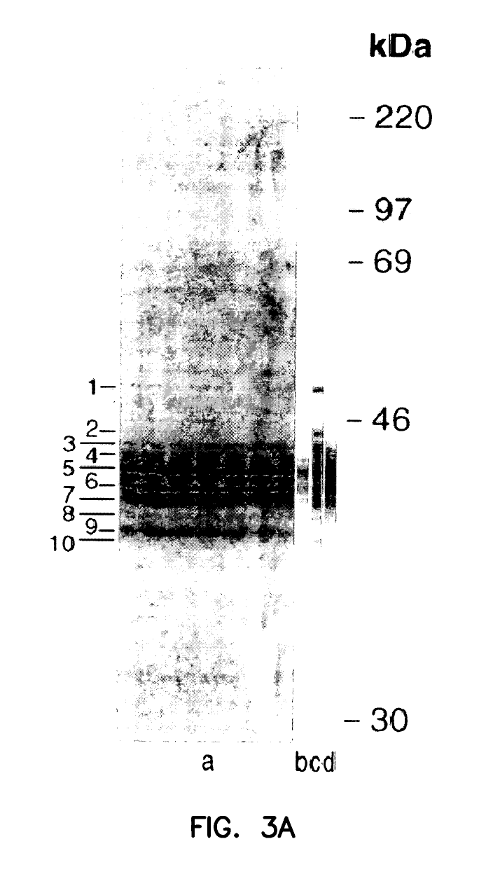

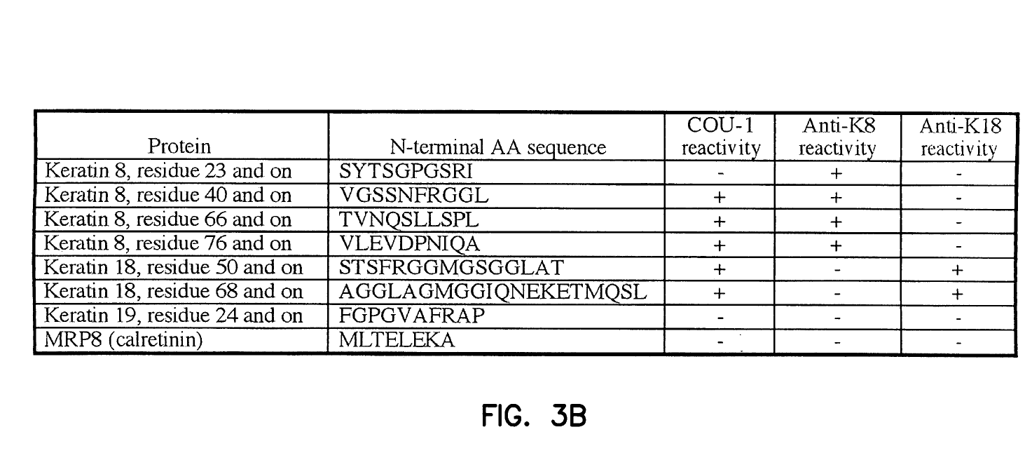

To determine the nature of the different K8/K18-like proteins found in the colon cancer tissues, an improved separation of the individual protein bands of the purified cytokeratin preparations was used. Purified cytokeratin preparations from different colon cancer tissues were, therefore, individually separated on large 14% SDS-PAGE gels, the proteins were blotted onto filters and Coomassie stained. Strips of the blot were incubated with either the anti-K8 antibody M20, the anti-K18 antibody CY-90, or the COU-1. FIG. 3A exhibits a typical blot of a colon cancer tissue sample, displaying approximately 10 different bands visualized by Coomassie staining. At this increased separation, 5 bands showed clear COU-1 reactivity. Additional bands, not stained with COU-1, were stained either with the anti-K8 antibody, the anti-K18 antibody or both (FIG. 3A).

All ten bands were N-terminal sequenced. As shown in FIG. 3B, the bands corresponded to different forms of K8, K18 and K19, except for one band that was identified as migration inhibitory factor-related protein 8 (MRP8, also known as calretinin), a calcium binding protein that may bind to cytokeratins (40). Most of the bands were N-terminally truncated K8 or K18, as demonstrated by the identified amino acid sequence starting at residue 23 to 76, instead of at the expected residue 1.

The amino terminal truncations of K8 corresponded to residues 23, 40, 66, and 76, while the truncations of K18 corresponded to residues 50 and 68. Significantly, the K8 and K18 truncations were found at the same residue in three different colon cancers, indicating that the truncations were caused by specific proteases.

Analysis of the sequence surrounding the cleavage site suggested that at least two different proteases were responsible for the cleavage, including one trypsin-like protease. The bands recognized by COU-1 were N-terminally truncated K8 and K18. Interestingly, however, not all the N-terminal truncated K8 and K18 proteins were recognized by COU-1. For example, no COU-1 binding was observed to an N-terminally truncated K8 protein where the first 22 amino acids were missing, nor did the antibody react with intact K8 or K18. The latter two observations were made by staining with anti-K8 and anti-K18 antibodies, respectively (bands 1 and 3 in FIG. 3A), but not by N-terminal sequencing, because the proteins were N-terminally blocked (K18 contains an acetylated serine at its N-terminus).

Mapping the COU-1 Epitope using Recombinant K8 and K18 Fragments

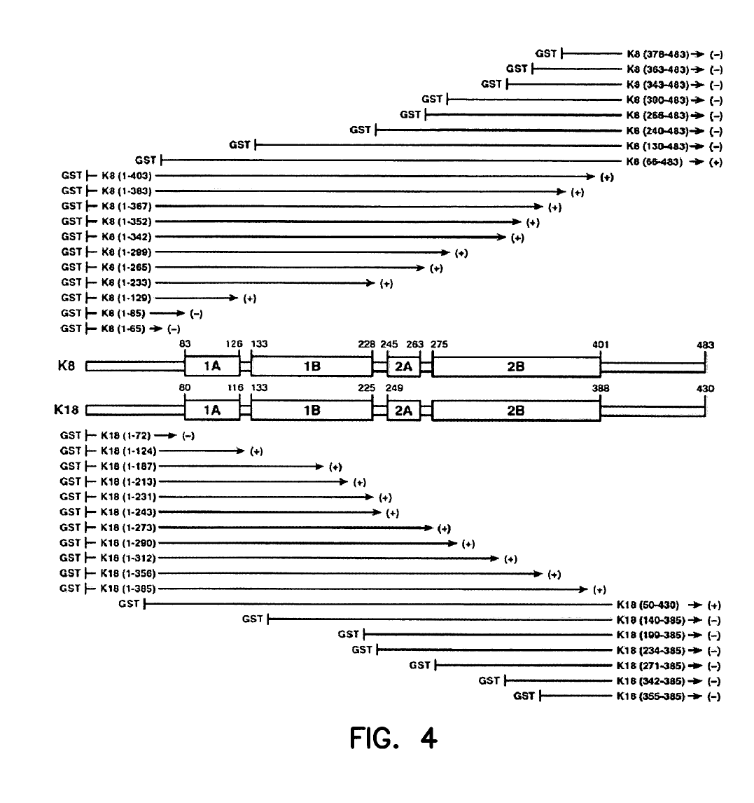

To detail the nature of the epitope recognized by COU-1, this epitope was mapped using a panel of recombinant N- or C-terminally-deleted K8 and K18 fragments or intact K8 and K18 expressed as GST-fusion proteins. The nature of these fragments is depicted in FIG. 4.

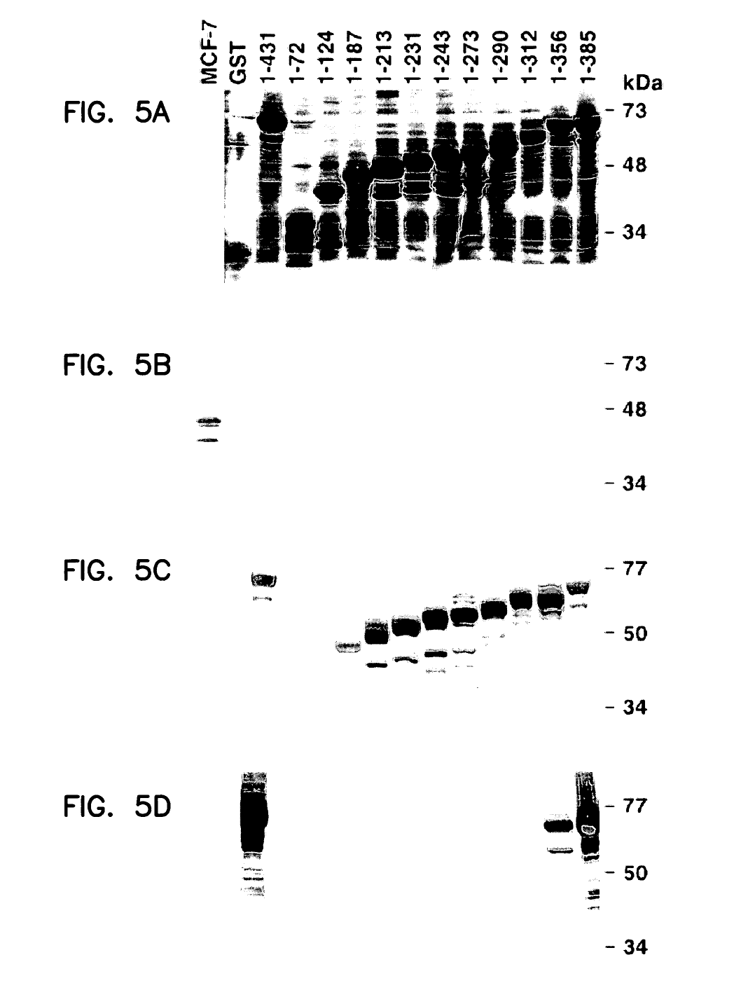

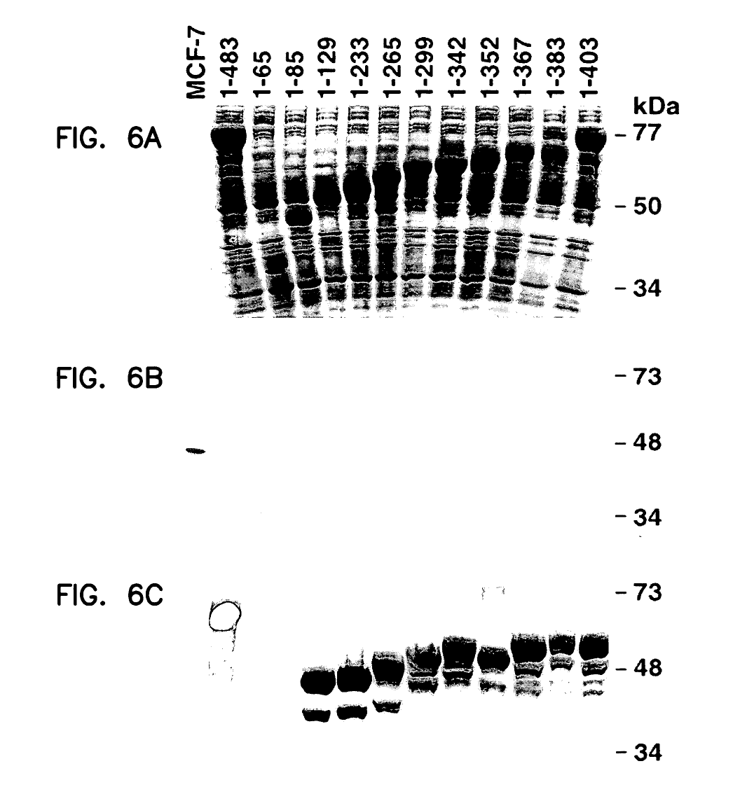

Initially, the panel of K8 and K18 fragments were separated by SDS-PAGE and blotted onto PVDF membranes. Subsequent analyses of these Western blots, surprisingly, showed that the COU-1 antibody did not bind to any of the individual K8 or K18 fragments. Nor did the COU-1 antibody bind to the intact K8 or K18 molecules (FIGS. 5B and 6B).

In each experiment, MCF7 cell lysate was included as a positive control, providing positively reacting bands at molecular weights of 42-46 kDa. To assure that the K8 and K18 fragment were evenly expressed, gels containing the fragment panel were run in parallel and the gels for the Western blots were stained with Coomassie blue (FIG. 6A). Moreover, blots of SDS-PAGE-separated K8/GST or K18/GST fusion proteins were stained with an anti-GST antibody (FIG. 5A). The results demonstrated an approximately even expression of the different fusion proteins, and that the lack of signal with COU1 was not due to a low expression level of the cytokeratin fragments or to incomplete transfer of proteins.

In addition, Mabs to K8 and K18, respectively, were tested for binding to the panel of K8 or K18 fragments. As shown in FIG. 5D, the anti-K18 Mab reacted strongly with K18(1-356), K18(1-385) and intact K18, but not with K18(1-312), indicating that its epitope was located in the region 312-356. Next, cytokeratins K8 and K18 complexes were tested to see whether those complexes were recognized by COU-1. Western blots of the panel of K18 fragments were incubated with the intact purified K8 and the unbound K8 was washed away before staining with COU-1 (FIG. 5C). COU-1 bound strongly to complexes formed between intact K8 and the K18 fragments K18(1-213) through K18(1-385). In contrast, COU-1 bound only weakly to intact K8/K18(1-187) and intact K8/intact K18, and no binding was observed to intact K8/K18(1-65) and intact K8/K18(1-124) (FIG. 5C).

Likewise, blots containing the panel of K8 fragments were incubated with intact K18 before staining with COU-1. COU-1 bound strongly to complexes formed between the intact K18 and the K8 fragments K8(1-213) through K8(1-385). In contrast, COU-1 bound weakly to intact K8/intact K18 and no binding was observed for K8(1-65)/intact K18 complexes (FIG. 6C).

N-terminal sequencing demonstrated that both K8 and K18 proteins from colon cancer patients were truncated. Experiments were performed to identify the K8/K18 heterotypic epitope bound by COU-1. In parallel, Western blots containing the C-terminal-deleted fragments surrounding the COU-1 epitope, K18(1-72), K18(1-124), K18(1-187) and intact K18 were generated. These blots were then incubated with one of the K8 fragments surrounding the COU-1 epitope, K8(1-85), K8(1-129) or K8(1-233), or the intact K8 protein. After permitting K8-K18 complex formation, the blots were incubated with COU-1 antibodies.

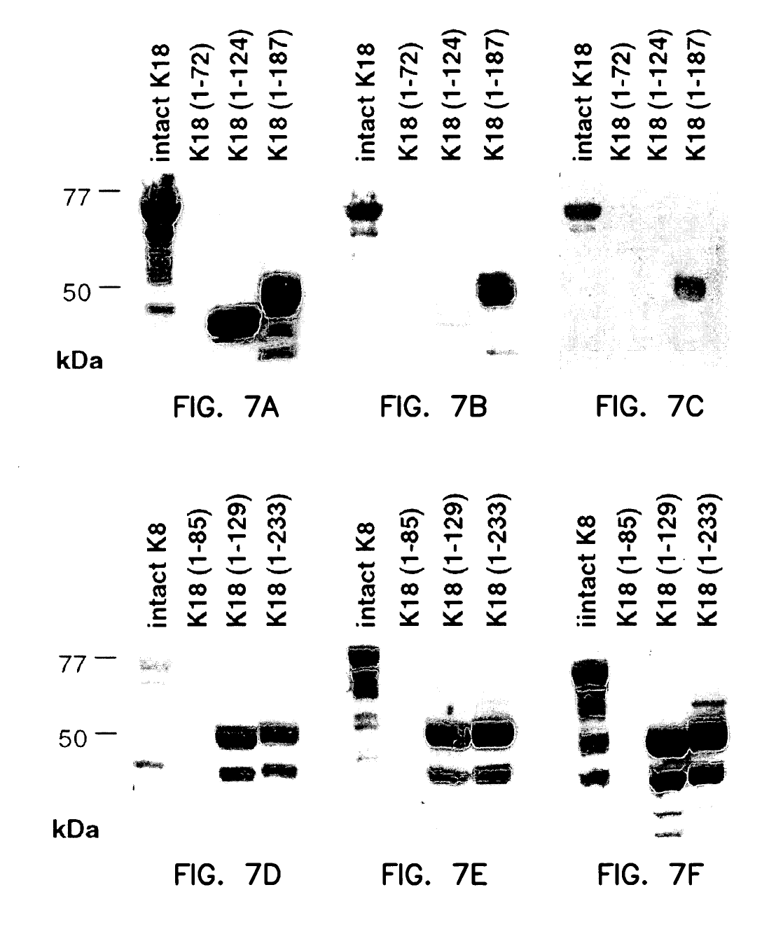

As shown in FIG. 7 (A-C), the epitope recognized by COU-1 is not exposed, or is only minimally exposed, on K18(1-124)/intact K8 or K18(1-124)/K8(1-233) complexes. In contrast, strong binding of COU-1 was observed for K18(1-124)/K8(1-129) complexes. No COU-1 binding was observed for any of the heterotypic complexes containing K8(1-85) or K18(1-72).

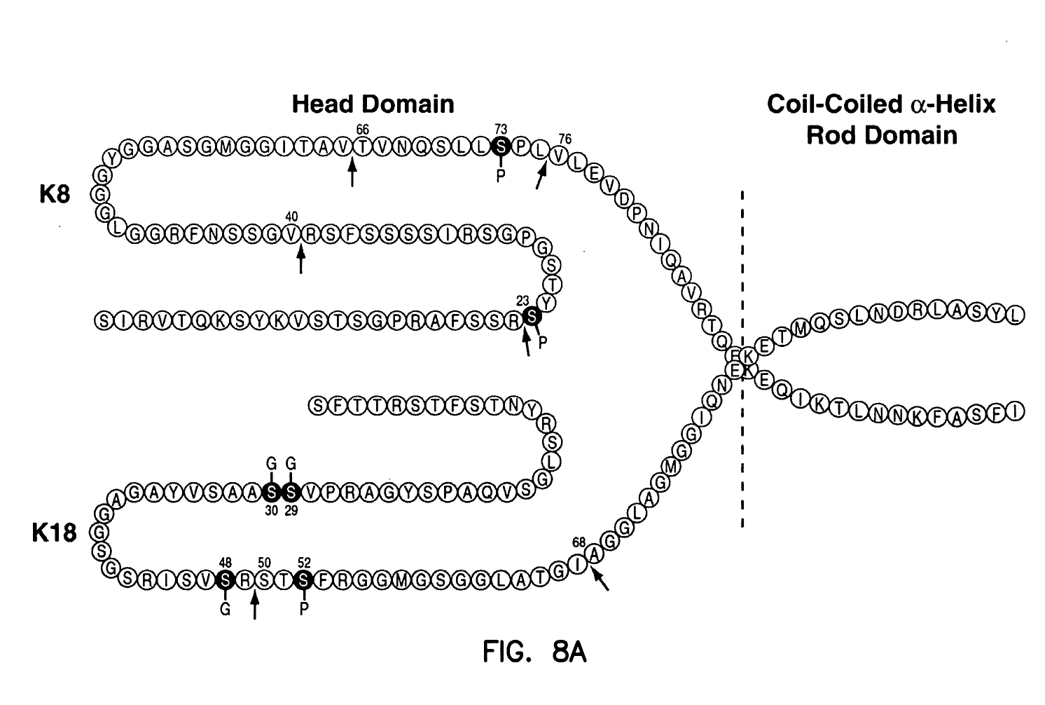

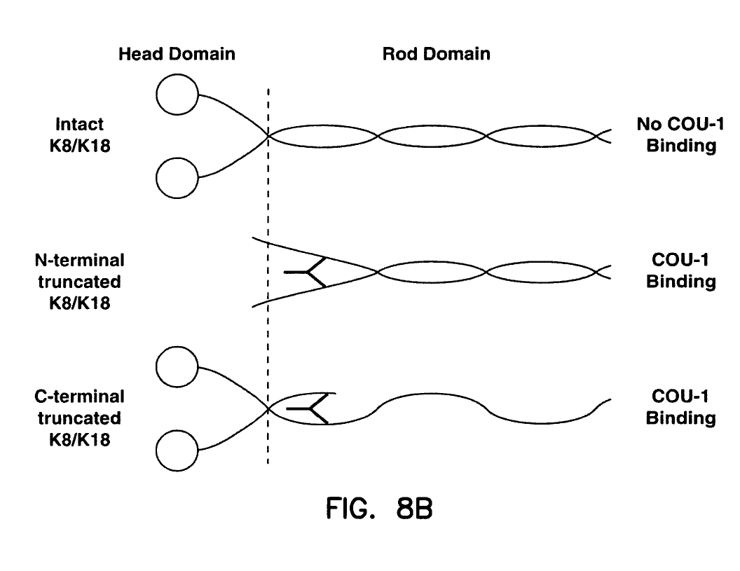

Taken together, these results confirm that the epitope recognized by COU-1 involves the K8 region 85-129 and the K18 region 72-124. As shown in FIGS. 4 and 8, this region involves the C-terminal part of the N-terminal head domain and the N-terminal part of the first helical domain, 1A, of the alpha-helical rod domain of both K8 and K18.

The results further demonstrate that this epitope is poorly exposed on heterotypic complexes of intact K8 and K18, even when intact K8 is complexed with K18(1-124). The COU-1 epitope is revealed when the first domain, A1, of the alpha helical rod is not in its normal coil-coil structure. This can be caused by truncation that removed essential contact points for the existing association leaving the COU-1 binding region of the K8/K18 complex in an unfolded state.

The combination described above was reversed such that Western blots of the C-terminal deleted fragments of K8(1-85), K8(1-129), K8(1-233) and intact K8 were incubated with the fragments of K18 surrounding the COU-1 epitope, K18(1-72), K18(1-124), K18(1-187) and intact K18. These blots were then incubated with the COU-1 antibody. As shown in FIG. 7 (D-F), the epitope recognized by COU-1 was equally exposed when K8(1-129) was complexed with K18(1-72), K18(1-124), and K18(1-187) or intact K18. Again, no COU-1 binding was observed with any heterotypic complexes containing K8(1-85).

COU-1 binding was tested using a panel of heterotypic complexes consisting of N-terminal deleted K8 and K18 combined with intact K8 and K18 using the heterotypic Western blot assay. These fragments were missing the first 129 amino acids or more as detailed in FIG. 4. However, no COU-1 binding was observed to any of these N-terminal-deleted heterotypic K8/K18 complexes, indicating that the COU-1 epitope was located within the N-terminal 129 amino acids (data not shown). The control showed that the N-terminal-deleted fragments were well recognized by the murine anti-K8 and anti-K18 Mabs.

The N-terminal sequencing data and the recombinant mapping data indicated that the COU-1 epitope were well exposed when the first 65 amino acids of K8 and the first 49 amino acids of K18 were missing.

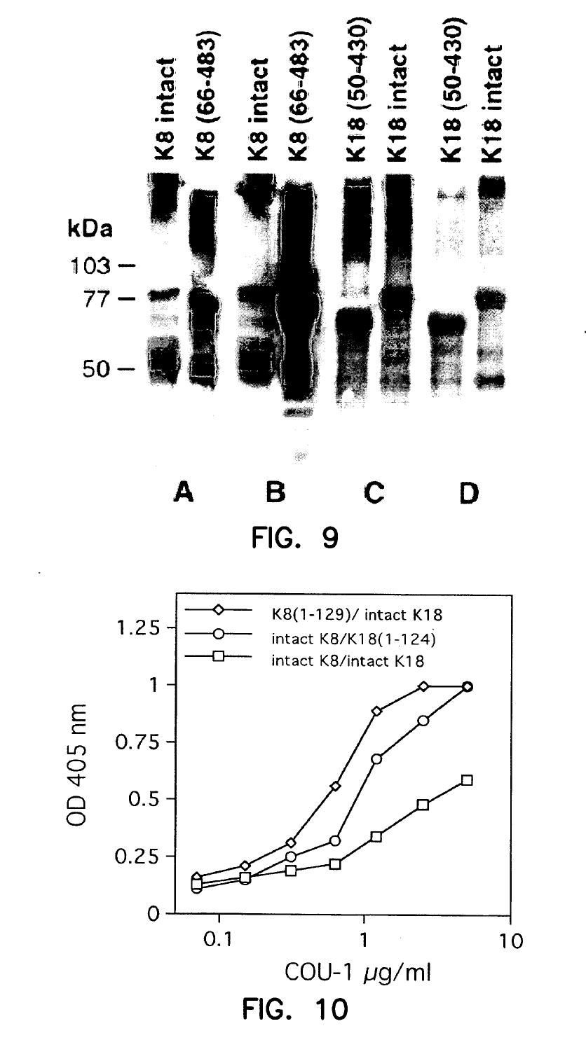

Two additional N-terminal deleted fragments, K8(66-483) and K18(50-430) were generated as GST fusion proteins. FIG. 9 shows blots of intact K8 and K8(66-483) incubated with K18(50-430) (A) or intact K18 (B). FIG. 9 also shows the blots of K18(50-430) and intact K18 incubated with K8(66-483) (C) or intact K8. Significantly stronger COU-1 binding was observed for K8(66-483)/K18(50-430) and K8(66-483)/intact K18 complexes than for intact K8/K18(50-430) or intact K8/intact K18 complexes.

Further investigations were made to determine whether the N-terminal cleavage of K8 and K18 observed in cancer cells might be caused by adenovirus infection. The adenovirus L3 23-kDa proteinase promotes specific cleaving of the N-terminal domain of K18, while leaving K8 intact in adenovirus infection of HeLa cells (41, 42). This cleavage resulted in removal of region 1-73 of the head-domain of K18 and the disassembly of the cytokeratin network into spheroid globules. Tests were performed to examine whether the fragmentation caused by adenovirus infection would result in the conformation change that allowed COU-1 binding.

Previous data indicated that COU-1 antibodies do not bind to K8/K18 from HeLa cells. Cytokeratin from HeLa cells infected with adenovirus were purified and separated by SDS-PAGE, demonstrating a band at a molecular weight of 41 kDa, in accordance with previous reports. Incubation of COU-1 with Western blot of the adenovirus-infected HeLa cells resulted in no staining (data not shown), suggesting that the cytokeratin fragments found in the adenocarcinomas were not a result of adenoviruis infection.

It seemed clear that the COU-1 epitope was only present when heterotypic K8/K18 complexes were formed. The epitope is not present on individual K8 and K18 molecules. However, the question remained as to why COU-1 binding to Western blots of SDS-separated cancer cell lysate was observed where the K8/K18 complexes may have dissociated. A possible explanation was that during the incubation steps, part of the different cytokeratins dissociate from the membrane and subsequently attach to and form high affinity heterotypic complexes with its complementary cytokeratin still bound to the membrane.

To examine this hypothesis, Western blots of lysate of the colon cancer cell line, colon 137, were separated into halves. One half was fixed with ethanol before incubation with the antibodies, while the other half was processed as usual without fixation. Staining was observed with anti-K18 antibody (CY-90) on both the fixed and the unfixed blots, while staining with COU-1 was only observed on the unfixed blot.

Earlier immunohistochemical studies had showed that ethanol fixation of tissue sections had no effect on the COU-1 antigen. Dot blots of the cancer lysate were tested for detection of the cancer-associated epitope with or without fixation. Staining with COU-1 was observed both with and without fixation, confirming that the COU-1 epitope was not affected by ethanol treatment. In conclusion, it seems the initial hypothesis was correct, i.e. that heterodimer formation of cytokeratins takes place during the development of Western blots and that such heterodimer formation by partially truncated cytokeratin is required for the formation of the COU-1 epitope.

COU-1 binding to the different recombinant heterotypic K8/K18 complexes was also measured by ELISA. Purified recombinant fragments of K8 or intact K8 were combined with purified recombinant fragments of K18 or intact K18 in a molar ratio of 1:1 to generate heterotypic complexes in urea. The samples were then dialyzed against PBS to allow the formation of the heterotypic complex, and coated at 5 μg/ml on ELISA plates. Intact K8 was combined with K18(1-124), K18(1-187), K18(1-213), and intact K18. In addition, intact K18 was combined with K8(1-65), K8(1-85), K8(1-129), and K8(1-233).

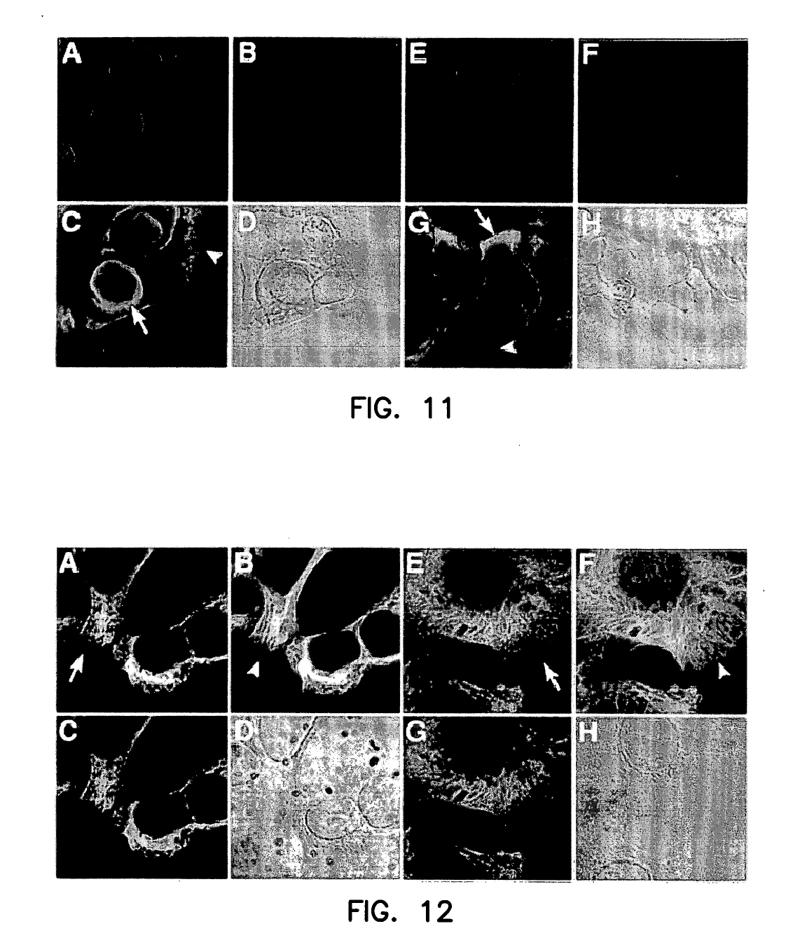

COU-1 bound with various intensity to all the complexes in this ELISA assay, except to K8(1-65)/intact K18 and intact K8(1-85)/intact K18 complexes. These data are in accord with the results from Western blot analysis. FIG. 10 shows the titration of COU-1 on three of the heterotypic complexes, demonstrating significantly stronger binding to the fragmented K8/K18 than to the intact K8/K18 complexes.

The kinetic parameters for the binding of COU-1 to different recombinant heterotypic K8/K18 complexes were measured by real-time biospecific interaction analysis (BIAcore). COU-1 exhibited high affinity binding to the heterotypic complexes of K8(1-124)/intact K18 and K8(1-124)/K18(1-124). The kinetic parameters for K8(1-124)/intact K18 were kₒₙ=1.7×10⁵ M⁻¹s⁻¹, k_{off}=1.2×10⁻⁴ s⁻¹, with derived association (Ka) and dissociation constants (Kd) of 1.4×10⁹ M⁻¹ and 7.1×10⁻¹⁰ M. The binding of COU-1 to K8(1-124)/K18(1-124) was slightly lower with kₒₙ=2.8×105 M⁻¹s⁻¹, k_{off}=3×10⁻⁴ s⁻¹, with derived Ka of 9.5×10⁸ M⁻¹ and Kd of 1.5×10⁻⁹M. In contrast, COU-1 exhibited an approximately 100-fold lower binding to intact K8/intact K18 with kₒₙ=9.1×10³ M⁻¹s⁻¹, k_{off}=5.0×10⁻⁵ s⁻¹, and Ka and Kd of 1.8×10⁷ M⁻¹ and 5.5×10⁻⁸ M, respectively.

Cellular Distribution of Truncated Heterotypic K8/K18 Complexes

To evaluate the cellular distribution of normal K8 and K18 compared to truncated K8/K18 heterotypic complexes, breast and colon cancer cell lines MCF-7 and BrCa01 were co-stained with COU-1 and either Mab M20 (anti-K8) or Mab CY-90 (anti-K18) and an analyzed by high resolution confocal microscopy (FIGS. 11 and 12). Mabs M20 and CY-90 both stained long fibers of intermediate filaments forming complex interconnecting networks. The fibers emanate from a perinuclear ring, from which the filaments appear to connect to the nuclear surface and extend throughout the cytoplasm, terminating at the plasma membrane. In contrast, COU-1 exhibited a speckled pattern, with staining of short filament-fragments and rod-like particles, indicative of fragmented intermediate filaments.

Examining the staining pattern of MCF7 cells within cell clusters, only the peripheral, newly-formed, proliferating cells were strongly positive for COU-1, while all cells were stained with anti-K18 and anti-K8 Mabs (FIG. 11). Within the proliferating cells of a cluster, COU-1 staining was must prominent at the outward cell surface, facing away from the cluster. In contrast, Mabs M20 and CY-90 stained the intermediate filamentous network throughout the cells. The speckled COU-1 staining was seen in close association to the intact intermediate filament network, as determined by overlay of images stained with COU-1 and Mab M20 or Mab CY-90 (FIG. 12).

Accordingly, N-terminally truncated forms of K8/K18 complexes were identified only in cancerous epithelia, whereas intact K8/K18 complexes were observed in both normal and cancerous simple glandular epithelia. The cleavage of both cytokeratin K8 and cytokeratin K18 at identical sites in different cancer patients indicates that specific proteases are involved. The cleavage sites at amino acids 22 and 40 on K8, and at amino acid 50 on K18, all contained the (S/F/V)XSR↓X(S/V) (SEQ ID NO:50) consensus sequence, suggesting that the enzyme responsible for these cleavages is a trypsin-like protease (FIG. 8). Analysis of the amino acid sequences in the vicinity of the cleavage sites revealed one other site on K8 that had the same general sequence (amino acid 32, GSR↓l (SEQ ID NO:64), but was not cleaved. This suggests that the amino acids at P3 or P1′ positions of the substrate are also influencing the recognition by this protease.

A consensus sequence was not apparent at the three remaining cleavage sites on K8 and K18 (TAV↓T (SEQ ID NO:51), SPL↓V (SEQ ID NO:52), TGI↓A (SEQ ID NO:53)). A protease that requires less stringent recognition conditions or several different proteases may be responsible for these cleavages. One such protease may be elastase-type protease that accepts valine, leucine and isoleucine in the P1 position.

It is unlikely that cleavage of cytokeratins K8 and K18 fragments occurred during the purification of cytokeratin from the tissue samples for several reasons. First, a cocktail of five enzyme-inhibitors was present at all times. Second, cytokeratin fragments were not observed following purification of cytokeratin from normal colon epithelia using identical purification conditions. Third, the HMab COU-1, which only recognizes the truncated form of K8/K18, can detect its epitope in cancerous, but not in normal, epithelia when tissue samples were minimally handled and immediately fixed.

In contrast to the earlier views, the maintenance of the cytokeratin network in epithelial cells is a dynamic process involving constant restructuring by assembly and disassembly of intermediate bundles (45). Microinjection of biotin-labeled cytokeratin or transfection with fluorescence-labeled cytokeratin has demonstrated an inward-directed flow of diffuse material at the cell periphery moving in the form of dots and thin filaments towards the deeper cytoplasm, where it coalesces with other filaments and filament bundles (46). While this process occurs in both normal and malignant epithelia cells, the results provided by the invention indicate the presence of a second degradation pathway specifically within cancer cells.

Also according to the invention, the human antibody, COU-1, cloned from a tumor-draining lymph node of a colon cancer patient, specifically recognizes the N-terminal truncated form of K8 and K18 when the two cytokeratins formed a heterotypic complex. Previous analysis of COU-1 indicated selective reaction of COU-1 with K18 (35, 48), or a modified K18 (31, 32, 49). Proteolytic cleavage of K18 in association with apoptosis has been reported (56). However, the cleavage sites for the apoptotic proteases, caspase-3,-6 and -7, are located in the conserved L1-2 linker and in C-terminal tail domain, and quite distant to the N-terminal cleavage sites, as we have studied in vital tumor tissue (56). Recently, an antibody (M30) was reported to recognize a neoepitope only exposed in apoptotic cancer cells and not vital or necrotic cells (57). The neoepitope become exposed when the C-terminus tail domain was liberated after cleavage by caspase-3,-6 or -7 into 26, 22 and 19 kDa fragments. The cleavage sites observed in colon cancer cells were also different from the one reported for adenovirus infected HeLa cells, where the N-terminal 73 amino acids of K18 were removed (41, 42). Surprisingly, no COU-1 binding to cleaved K8/K18 heterotypic complexes from infected HeLa cells was observed, while COU-1 bound K8/K18 complexes where the 67 most N-terminal amino acids of K18 were removed. This suggests, although the cleavage sites seem close, additional removal of 6 amino acids may cause conformational changes that prevent COU-1 from binding.

Some evidence indicates that K8/K18 is intimately associated with cell migration and invasiveness. N-terminal cleavage of K8/K18 may influence these processes. Moreover, the missing N-terminal head domain of K8/K18 contains several important phosphorylation sites, including ser52 on K18, which has been associated with filament reorganization and compartment localization and a second phosphorylation site important for binding to the 14-3-3 protein (58, 59). In K8 the phosphorylation site ser23 has been associated with mitogen activation (60).

The abbreviations used herein are: K8, cytokeratin 8; K18, cytokeratin 18; IF, intermediate filaments; HMab, human monoclonal antibody; FCS, fetal calf serum; AP, alkaline phosphatase; QFF, Q-Sepharose fast flow; ELISA, enzyme-linked immunosorbent assay; PBS, phosphate-buffered saline; TBS, Tris-buffered saline; PVDF, polyvinylidene difluoride membranes; FITC, fluorescein isothiocyanate.

### EXAMPLE 3

### Cloning Antibody Fragments that Bind the Cancer-Associated Epitope

To further develop antibodies useful for detection of cancer, nucleic acids encoding portions of antibodies were cloned and screened by phage display selection for binding to the cancer-associated epitope of the invention. These nucleic acids encode human Fab and other fragments.

Materials and Methods

Antibodies. The human monoclonal IgM antibody, COU-1, is secreted by the hybridoma cell line, B9165, derived by fusing the human lymphoblastoid cell line WI-L2-729-HF2 and lymphocytes obtained from mesenteric lymph nodes from a patient with colon cancer as described above. See also Borup-Christensen, P., Erb, K., Jensenius, J. C., Nielsen, B. & Svehag, S. E. (1986) Int. J. Cancer 37, 683-688. The human-human hybridoma cell line was grown in protein free medium: RPMI 1640 medium (GIBCO, Grand Island, N.Y.) supplemented with SSR3 serum replacement (Medicult, Copenhagen, Denmark). The COU-1 antibody was purified from cell culture supernatant by affinity chromatography on Sepharose-coupled murine monoclonal anti-human μ chain antibody (HB57, American Type Culture Collections, Rockville, Md.). The antibody was eluted with 0.1 M diethylamine, pH 10.5, followed by fractionation by FPLC. IgM purified from normal human serum (Cappel, Cochranville, Pa.) was used as a control.

The human monoclonal IgM antibody, 16.88 was obtained from Dr. R. McCabe. See Haspel, et al., (1985) Cancer Res. 45, 3951-3961. This antibody has been used successfully for tumor imaging in humans. See Steis et al. (1990) J. Clin. Oncol. 8, 476-490; Boven et al. (1991) Eur. J. Cancer 27, 1430-1436; Rosenblum et al. (1994) Cancer Immunol. Immunother. 39, 397-400). Two murine monoclonal antibodies, M20 directed against normal cytokeratin 8 and CY-90 directed against normal cytokeratin 18, were obtained from Sigma Chemical Co. (St. Louis, Mo.).

PCR amplification and cloning of the variable heavy and light chain genes. Total RNA was prepared from the B9165 hybridoma cell line by the guanidinium method. After reverse transcription, the μ (Fd region) and κ chains were amplified by the polymerase chain reaction (PCR) using a set of family-specific primers using methods described in Persson et al., (199 1) Proc. Natl. Acad. Sci. USA 88, 2432-2436. The amplified light chain DNA was cut with the restriction enzymes Sac I and Xba I and ligated with Sac I/Xba I-linearized pComb3 vector for 3 h as described in Burton et al., (1991) Proc. Natl. Acad. Sci. USA 88, 10134-10137, and Barbas et al. (1991) Proc. Natl. Acad. Sci. USA 88, 7978-7982. The ligated material was purified and transformed by electroporation into 200 μl Escherichia coli XLI-Blue cells. After transformation, the cells were grown overnight and phagemid DNA was prepared.

Subsequently, the PCR amplified heavy chain and isolated phagemid DNA containing the light chain were digested with the restriction enzymes Spe I and Xho I. The heavy chain phagemid fragments were ligated and used to transform XLI-Blue. The Fab library was grown in SOC medium for 1 h at 37° C. following addition of SB medium containing carbenicillin (50 μg/ml) and tetracycline (10 μg/ml). After 3 h, helper phage VCS-M13 (10¹² plaque-forming units) was added and the culture was shaken for an additional 2 h. Kanamycin (70 μg/ml) was added and the culture was incubated at 30° C. overnight. The supernatant was cleared by centrifugation (4000×g for 20 min) at 4° C. Phage were precipitated by a second round of centrifugation after the addition of 5% polyethylene glycol and 0.15 M NaCl and incubation on ice for 30 min. Phage pellets were resuspended in phosphate-buffered saline, pH 7.4 (PBS) containing 1% (w/v) bovine serum albumin (BSA) and centrifuged for 3 min at 10,000×g to pellet debris.

Enrichment of antigen-binding phage through panning. Panning of the B9165 antibody library was carried out using methods described in Burton et al. (1991) Proc. Natl. Acad. Sci. USA 88, 10134-10137. In brief, microtiter wells were coated overnight with ultrasonicated lysate of a colon cancer cell line (colo 137) in 0.1 M bicarbonate buffer, pH 8.6 at 4° C. See Ditzel et al. (1992) Eur. J. Nucl. Med. 19, 409-417. Following blocking with PBS containing 3% BSA for 1 h at 37° C., 50 μl phage suspension in PBS was added to each well and incubated for 2 h. Unbound phage were removed by vigorous washing 10 times with PBS containing 0.05% (w/v) Tween 20 (PBS-Tween)(Merck, Darmstadt, FRG). Bound phage, enriched for those bearing antigen-binding Fabs, were eluted with 0.2 M glycine/HCl, pH 2.2. The eluted phage were amplified by infection of E. coli and recovered by superinfection with VCS-M 13 helper phage. The panning procedure was carried out twice. Phagemid DNA was isolated from the last round of panning, cut with NheI and SpeI and religated. This step excised the cpIII gene, resulting in a vector producing soluble Fab fragments.

ELISA analysis of B9165 Fab and intact antibodies. Fabs were prepared as bacterial supernatants through a freeze-thawing procedure, using methods reported by Burton et al. (1991) Proc. Natl. Acad. Sci. USA 88, 10134-10137, and Barbas et al. (1991) Proc. Natl. Acad. Sci. USA 88, 7978-7982.

To assess specificity, supernatants and purified Fabs were screened in an ELISA system for binding to ultrasonicates of colon cancer cells (Colon 137), BSA (Sigma), ovalbumin (Sigma), recombinant HIV-1 gp120 (IIIB) (Intracel, Issaquah, Wash.) and human placental DNA (Sigma). ELISA wells (Costar) were coated overnight at 4° C. with 50 μl of antigen (1-10 μg/ml) in 0.1 M bicarbonate buffer, pH 8.6. DNA in PBS was dried on the ELISA wells at 37° C. The wells were washed twice with PBS, blocked by filling the wells with 3% BSA in PBS for 1 h at 37° C., and incubated with human Fab samples or intact human IgM antibody for 2 h at 37° C. Plates were washed 10 times with PBS-Tween and bound Fab was detected with alkaline phosphatase (AP) labeled goat anti-human IgG F(ab′)₂ (Pierce Chemical Co, Rockford, Ill.) diluted 500 fold in PBS or alkaline phosphatase-labeled rabbit anti-human κ-chain (Sigma) diluted 1000 fold in PBS. Bound antibody was visualized with para-nitrophenylphosphate (Sigma)(1 mg/ml, 1 mM MgCl₂, 10% (w/v) diethanolamine, pH 9.6) and read at 405 nm.

Purification of Fab. Recombinant B9165 Fab was purified using methods described in Ditzel et al. (1995) J. Immunol. 154, 895-908 with some modifications. In brief, E. coli containing the appropriate clone was inoculated into one liter cultures of superbroth containing carbenicillin (50 μg/ml), tetracycline (10 μg/ml) and MgCl₂ (20 mM), and grown at 37° C., with shaking, for 6 h. Protein expression was then induced with 2 mM isopropyl β-D-thiogalactopyranoside and growth continued at 30° C. overnight. Soluble Fab was purified from bacterial supernatants by affinity chromatography using a goat antibody against human IgG F(ab′)2 (Pierce) cross-linked to protein G gammabind matrix (Pharmacia). The column was washed with PBS and bound Fab eluted with 0.2 M glycine/HCl, pH 2.2, and immediately neutralized with 1 M Tris/HCl, pH 9.0.

Nucleotide sequencing. Sequencing was carried out on a 373A automated DNA sequencer (ABI, Foster City, Calif.) using a Taq fluorescent dideoxy terminator cycle sequencing kit (ABI). Primers for the elucidation of light chain sequence were the SEQKb primer (5′-ATAGAAGTTGTTCAGCAGGCA-3′, SEQ ID NO:41), hybridizing to the (+) strand and the KEF primer (5′-GAATTCTAAACTAGCTAGTTCG-3′, SEQ ID NO:42) hybridizing to the (−) strand. For the heavy chain, the CMHD primer (5′-CAAGGGCTTGAGTGGATGGGA-3′, SEQ ID NO:43) and the T3 primer (5′-ATTAACCCTCACTAAAG-3′, SEQ ID NO:44) were used, binding to the (−) strand.

Analysis by confocal laser scanning microscopy. Human colon cancer cell-lines (H3619 and colo 137), and breast cancer cell-lines (MCF-7 and H3396) were grown in Iscove's modified Dulbecco's medium containing 10% FBS and allowed to adhere to chambered coverslips (Nunc, Kamstrup, Denmark) for 48 h at 37° C., 5% CO₂, in order to form monolayers. Experiments were performed using the primary COU-1 antibodies, B9165 Fab, murine anti-cytokeratin 8, murine anti-cytokeratin 18, and HuMab 16.88 as indicated below. All antibodies were tested at 10 μg/ml except B9165 Fab (30 μg/ml).

1) Intracellular staining. H3619 and colo 137 cells were permeabilized with methanol at −20° C. for 5 min, blocked with normal goat serum followed by incubation with primary antibodies at room temperature for 1 h. The cells were then washed 3 times with culture medium and incubated with FITC-labeled goat anti-human κ-chain antibody (Southern biotech) or FITC-labeled goat anti-mouse IgG (BioSource) diluted 1:100 and 1:50 respectively in PBS for 1 h at room temperature.

2) Surface staining. Live H3619 cells were incubated with COU-1 antibodies at 4° C. for 2 h, washed 3 times with cold culture medium and incubated with secondary FITC-labeled antibody at 4° C. for 1 h.

3) Internalization. Live H3619 and colo 137 cells were incubated with COU-1 antibodies or B9165 Fab at 37° C. for 6 h, followed by washing 3 times and permeabilization with methanol at −20° C. for 5 min. Cells were blocked with normal goat serum and incubated with secondary FITC-labeled antibody at RT for 1 h. For all experiments, following primary and secondary antibody incubations, the cells were washed, fixed with 2% paraformaldehyde in PBS for 15 min at room temperature, washed twice and mounted in anti-fading reagent (30 mM dithioerythritol:PBS:glycerol, 2:9:1). Staining of cells was evaluated by confocal laser scanning microscopy. As control all experiments were carried out omitting the primary antibody.

Immunohistochemical analysis. Tissue specimens were obtained from colorectal cancer patients undergoing surgical resection. Normal colon tissue was taken from the resectate approximately 15 cm away from the site of the tumor. Tissues were fixed in 96% alcohol for 6 h at 4° C. Afterwards, tissues were paraffin embedded and cut into 5 μm sections. Sections were deparaffinized in xylol, rehydrated through graded alcohol and washed in PBS-Tween. Sections were incubated for 2 h at room temperature in a humidified chamber with 100 μl of murine monoclonal antibody, human monoclonal IgM antibody or normal polyclonal human IgM, all at 0.5-10 μg/ml. The slides were washed and incubated with Aβ-labeled rabbit anti-human IgM (Dako, Glostrup, Denmark), horse-radish peroxidase (HRP) labeled rabbit anti-human IgM (Dako) or HRP-labeled rabbit anti-mouse IgG (Dako) diluted in PBS with 10% (w/v) bovine serum albumin for 1 h at room temperature. After washing, the HRP was visualized by development with chromogenic substrate (0.6 mg diaminobenzidine per ml PBS with 0.01% H₂O₂) and AP with 0.2 mg naphthol-AS-Mx phosphate (Sigma), 1 mg Fast Red TR Salt (Sigma), 20 μg dimethylformamide per ml 0.1M Tris/HCl, 1M levamisole, pH 8.2. The sections were counterstained with Mayer's haematoxylin, dehydrated in xylene and mounted in Aquamount (Gurr, Poole, England). The staining intensity was graded as follows: (−) no staining, (+) weak staining, (++) moderate staining, (+++) strong staining.

Results

Phage display expression and sequencing of HuMab that can bind the Cancer-Associated Epitope. RNA was extracted from the B9165 cell line and the heavy (μ Fd region) and light (κ)-chain genes from the corresponding cDNA were amplified by PCR using 3′ family specific primers and a 5′ constant primer. The light and heavy chain products were then sequentially cloned into the M13 phage surface expression vector pComb3 to generate a library of 2×10⁶ members. The phage library was selected twice on an ultrasonicate of the COU-1 antigen positive colon cancer cell line (colon 137). Eluted phage from the last round of selection were used to infect E. coli XLI-blue cells. DNA was prepared from these cells and gene III fragment removed by NheI/SpeI digestion and ligation. The reconstructed phagemids were used to transform XLI-Blue to produce clones secreting soluble Fab fragments. Supernatants of three of the 80 single Fab expression clones tested, exhibited binding to colon 137 lysate and no binding to ovalbumin in ELISA.

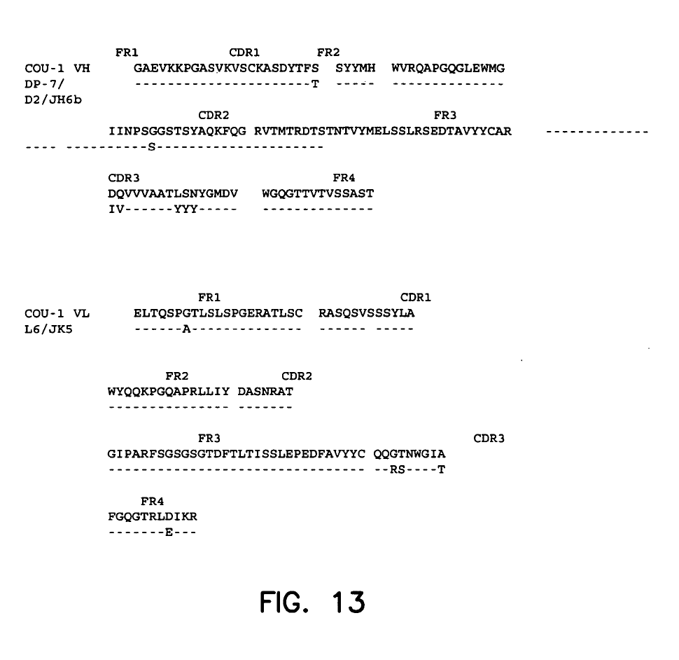

The sequences of these three clones were identical. Sequence analysis showed that the B9165 hybridoma cell light chain belongs to the VKIII family and that it exhibits 97% (269/276) nucleotide homology to L6 as closest germ-line (FIG. 13). The B9165 light chain contained an extra serine inserted corresponding to codon 30. The light chain J segment showed 95% (36/38) nucleotide homology to the germ-line JK5 segment. Further, sequence analysis showed that the heavy chain belongs to the VHI family, exhibiting 98% nucleotide homology to the heavy chain germ-line Dβ-7. The heavy chain J segment showed 96% (53/55) nucleotide homology to the germ-line JH6b segment. The D segment of COU-1 showed closest homology to the D2 germ-line D segment with a 16 nucleotide stretch of complete homology.

Purified recombinant B9165 Fab was tested in parallel with the intact COU-1 antibodies and normal polyclonal IgM for binding to lysate of colon cancer cells (colo 137) and irrelevant antigens in ELISA. The B9165 Fab and COU-1 exhibited strong binding to colon 137 lysate, but not to a panel of other antigens including BSA, ovalbumin, human DNA and HIV-1 gp120 (data not shown). In contrast, normal human IgM did not bind to any of the antigens. The concentration needed for saturation was significantly higher for the B9165 Fab (20 μg/ml) than for the intact antibody (1 μg/ml) and was similar to that previously measured for chemical derived half-monomeric fragments, exhibiting a Ka of 2×10⁶ M⁻¹ (Ditzel, H., Erb, K., Leslie, G. & Jensenius, J. C. (1993) Hum. Antibod. Hybridomas 4, 86-93).

COU-1 binds preferentially to malignant carcinoma cells. The subcellular localization of the antigen recognized by COU-1 in tissue biopsies of colon and rectal adenocarcinomas was studied using an indirect immunoperoxidase and alkaline-phosphatase techniques. At high magnification, distinct fibrillar staining of intermediated filaments by COU-1 was observed. In small cell clusters or individual cells, intense staining was seen at the periphery, possibly associated with the cell surface. In addition, enhanced staining associated with the junctional zone between adjacent cells was seen. No staining was observed in adjacent normal colon crypt epithelial cells in five of eight colon or rectal cancer. In the other three cancers, weak staining of a few individual cell surrounded by negative cells was observed in adjacent morphologically normal colon tissues in addition to strong staining of the cancer tissue. Although these colon epithelia looked morphologically normal, this may not be the case. Murine anti-cytokeratin 8 antibodies and anti-cytokeratin 18 antibodies (not shown) gave intense staining of the adjacent normal colon epithelia as well as of the colon cancer tissue. COU-1, however, reacted only with the malignant cells and not with the normal epithelia. A comparison of the staining levels for COU-1, murine anti-cytokeratin 8 and 18, and 16.88 is given in Table 3. The 16.88 antibody showed strong staining of the colon cancer cells, weak staining only in some areas of the normal colon epithelia, but in addition stained the smooth muscle fibers and myoepithelia derived connective tissue was observed (Table 5).

TABLE 5
Comparison of antibody reactivity with ethanol fixed
malignant and normal tissues.

| | HuMab | HuMab | Normal | Mab | Mab |
|---|---|---|---|---|---|
| Tissue\Antibody | COU-1 | 16.88 | human IgM | anti-K8 | anti-K18 |
| Colon Cancer | +++ | +++ | − | +++ | +++ |
| Normal epithelia | − | + | − | +++ | +++ |
| Connective tissue | − | −− | − | − | |
| Smooth muscle | − | ++ | − | − | − |
| Hepatocytes | − | − | − | +++ | +++ |
| Biliary ducts | +++ | +++ | − | +++ | +++ |

The antibodies were compared for staining of colon metastases in liver versus surrounding normal liver tissue. COU-1 gave intense staining of the metastasis whereas no staining of the majority of hepatocytes was observed. A few hepatocytes in the periportal zones were weakly positive. Similarly, the 16.88 antibody did not stain the majority of the hepatocytes. However, the myoepithelia connective tissue was stained by 16.88, but not with COU-1. Both human antibodies stained the biliary ducts. The murine anti-cytokeratin 8 and 18 (not shown) antibodies stained the metastases as well as the normal hepatocytes strongly and with equal intensity. The staining decreased towards the centrilobular area. Particular strong staining was seen associated with the cell membrane of the hepatocytes with the murine Mabs.

Phage display and bacterial expression was therefore used to clone and further characterize Fab and other antibody fragments from a hybridoma cell line expressing the human monoclonal antibody COU-1. The binding characteristics of the cloned B9165 Fab were very similar to previous reports for the half-monomeric fragments generated by chemical reduction and alkylation (Ditzel, H., Erb, K., Leslie, G. & Jensenius, J. C. (1993) Hum. Antibod. Hybridomas 4, 86-93). Sequence analysis showed that the variable region of the heavy and light chain had minimal somatic mutations with 98% and 97% nucleotide homology to the closest germ-line V genes, respectively. This is in accordance with COU-1 being an IgM antibody, and indicates that substantial affinity maturation through site directed mutagenesis is possible.

The foregoing specification, including the specific embodiments and examples, is intended to be illustrative of the present invention and is not to be taken as limiting. Numerous other variations and modifications can be effected without departing from the true spirit and scope of the present invention.

References

The following references, to the extent that they provide exemplary procedural or other details supplementary to those set forth herein, are specifically incorporated herein by reference.
1. Schmidt, A., Heid, H. W., Schäfer, S., Nuber, U. A., and Zimbelmann, R. Desmosomes and cytoskeletal architecture in epithelial differentiation: cell type-specific plaque components and intermediate filament anchorage. European Journal of Cell Biology, 65: 229-245, 1994.
2. Fuchs, E. and Cleveland, D. W. A structural scaffolding of intermediate filaments in health and disease. Science, 279: 514-519, 1998.
3. Hatzfeld, M. and Franke, W. W. Pair formation and promiscuity of cytokeratins: formation in vitro of heterotypic complexes and intermediate-sized filaments by homologous and heterologous recombinations of purified polypeptides. J. Cell Biol., 101: 1826-1841, 1985.
4. Fuchs, E. and Weber, K. Intermediate filaments: structure, dynamics, function, and disease. Annu. Rev. Biochem., 63: 345-382, 1994.
5. Moll, R. Cytokeratins as markers of differentiation in the diagnosis of epithelial tumors. Subcell. Biochem., 31: 205-262, 1998.
6. Steinert, P. M. and Roop, D. R. Molecular and cellular biology of intermediate filaments. Ann. Rev. Biochem., 57: 593-625, 1988.
7. Hatzfeld, M. and Weber, K. The coiled coil of in vitro assembled keratin filaments is a heterodimer of type I & II keratins; use of site-specific mutagenesis and recombinant protein expression. J. Cell Biol., 110: 1199-1210, 1990.
8. Steinert, P. M. The two-chain coiled-coil molecule of native epidermal keratin intermediate filaments is a type I-type II heterodimer. J. Biol. Chem., 265: 8766-8774, 1990.
9. Traub, P. Intermediate filaments: a review, vol. ed. Heidelberg, Germany: Springer-Verlag, 1985.
10. Hatzfeld, M. and Weber, K. Tailless keratins assemble into regular intermediate filaments in vitro. J. Cell Sci., 97: 317-324, 1990.
11. Shoeman, R. L., Mothes, E., Kesselmeier, C., and Traub, P. Intermediate filament assembly and stability in vitro: effect and implications of the removal of head and tail domains of vimentin by the human immunodeficiency virus type I protease. Cell Biol. Int. Rep., 14: 583-594, 1990.
12. Bader, B. L., Magin, T. M., Freudenmann, M., Stumpp, S., and Franke, W. W. Intermediate filaments formed de novo from tail-less cytokeratins in the cytoplasm and in the nucleus. J. Cell Biol., 115: 1293-1307, 1991.
13. Hatzfeld, M. and Burba, M. Function of type I and type II keratin head domains: their role in dimer, tetramer and filament formation. J. Cell Science, 107: 1959-1972, 1994.
14. Ku, N. O., Michie, S., Oshima, R. G., and Omary, M. B. Chronic hepatitis, hepatocyte fragility, and increased soluble phosphoglycokeratins in transgenic mice expressing a keratin 18 conserved arginine mutant. J. Cell Biology, 131: 1303-1314, 1995.
15. Ku, N. O., Gish, R., Wright, T. L., and Omary, M. B. Keratin 8 mutations in patients with cryptogenic liver disease. N. Engl. J. Med., 344: 1580-1587, 2001.
16. Baribault, H., Price, J., and Oshima, R. G. Mid-gestational lethality in mice lacking keratin 8. Genes and Development, 7: 1191-1202, 1993.
17. Baribault, H., Penner, J., Iozzo, R. V., and Wilson-Heiner, M. Colorectal hyperplasia and inflammation in keratin 8-deficient FVB/N mice. Genes and Development, 8: 2964-2973, 1994.
18. Hesse, M., Franz, T., Tamai, Y., Taketo, M. M., and Magin, T. M. Targeted deletion of keratins 18 and 19 leads to trophoblast fragility and early embryonic lethality. EMBO J., 19: 5060-5070, 2000.
19. Bauman, P. A., Dalton, W. S., Anderson, J. M., and Cress, A. E. Expression of cytokeratin confers multiple drug resistance. PNAS, 91: 5311-5314, 1994.
20. Parekh, H. K. and Simpkins, H. The differential expression of cytokeratin 18 in cisplatin-sensitive and -resistant human ovarian adenocarcinoma cells and its association with drug sensitivity. Cancer Research, 55: 5203-5206, 1995.
21. Anderson, J. M., Heindl, L. M., Bauman, P. A., Ludi, C. W., Dalton, W. S., and Cress, A. E. Cytokeratin expression results in a drug-resistant phenotype to six different chemotherapeutic agents. Clinical Cancer Research, 2: 97-105, 1996.
22. Schaafsma, H. E., Ramaekers, F. C. S., van Muijen, G. N. P., Lane, E. B., Leigh, I. M., Robben, H., Huijsmans, A., Ooms, E. C. M., and Ruiter, D. J. Distribution of cytokeratin polypeptides in human transitional cell carcinomas, with special emphasis on changing expression patterns during tumor progression. Am. J. Pathology, 136: 329-343, 1990.
23. Moll, R. Cytokeratins in the histological diagnosis of malignant tumors. Int. J. Biol. Markers, 9: 63-69, 1994.
24. Moll, R., Franke, W. W., Schiller, D. L., Geiger, B., and Krepler, R. The catalog of human cytokeratins: Patterns of expression in normal epithelia, tumors and cultured cells. Cell, 31: 11-24, 1982.
25. Pankov, R., Umezawa, A., Maki, R., Der, C. J., Hauser, C. A., and Oshima, R. G. Keratin 18 activation by Ha-ras is mediated through ETS and Jun binding sites. PNAS, 91: 873-877, 1994.
26. Oshima, R. G., Abrams, L., and Kulesh, D. Activation of an intron enhancer within the keratin 18 gene by expression of c-fos and c-jun in undifferentiated F9 embryonal carcinoma cells. Genes. Dev., 4: 835-848, 1990.
27. Oshima, R. G., Baribault, H., and Caulín, C. Oncogenic regulation and function of keratins 8 and 18. Cancer and Metastasis Reviews, 15: 445-471, 1996.
28. Chu, Y. W., Runyan, R. B., Oshima, R. G., and Hendrix, M. J. C. Expression of complete keratin filaments in mouse L cells augments cell migration and invasion. PNAS, 90: 4261-4265, 1993.
29. Pankov, R., Simcha, I., Zöller, M., Oshima, R. G., and Ben-Ze'ev, A. Contrasting effects of k8 and k18 on stabilizing k19 expression, cell motility and tumorigenicity in the BSp73 adenocarcinoma. J. Cell Science, 110: 965-974, 1997.
30. Hendrix, M. J. C., Seftor, E. A., Chu, Y. W., Seftor, R. E. B., Nagle, R. B., McDaniel, K. M., Leong, S. P. L., and Yohem, K. H. Co-expression of vimentin and keratins by human melanoma tumor cells: Correlation with invasive and metastatic potential. J. Natl. Cancer Inst., 84: 165-174, 1992.
31. Ditzel, H. J., Garrigues, U., Andersen, C. B., Larsen, M. K., Garrigues, H. J., Svejgaard, A., Hellstrom, I., Hellstrom, K. E., and Jensenius, J. C. Modified cytokeratins expressed on the surface of carcinoma cells undergo endocytosis upon binding of human monoclonal antibody and its recombinant Fab fragment. PNAS USA, 94: 8110-8115, 1997a.
32. Ditzel, H. J., Rasmussen, J. W., Erb, K., Borup-Christensen, P., Titlestad, I., Lassen, E., Fenger, C., Kronborg, O., and Jensenius, J. C. Tumor detection with 131I-labeled human monoclonal antibody COU-1 in patients with suspected colorectal carcinoma. Cancer Research, 53: 5920-5928, 1993.
33. Chan, R., Rossitto, P. V., Edwards, B. F., and Cardiff, R. D. Presence of proteolytically processed keratins in the culture medium of MCF-7 cells. Cancer Research, 46: 6353-6359, 1986.
34. Hembrough, T. A., Vasudevan, J., Allietta, M. M., Glass, W. F.II, and Gonias, S. L. A cytokeratin 8-like protein with plasminogen-binding activity is present on the external surfaces of hepatocytes, HepG2 cells and breast carcinoma cell lines. J. Cell Science, 108: 1071-1082, 1995.
35. Borup-Christensen, P., Erb, K., Ditzel, H., Nielsen, B., Larsen, J. K., Svehag, S. E., and Jensenius, J. C. Human hybridoma producing monoclonal antibodies against colorectal cancer-associated antigens. APMIS, 98: 674-684, 1990.
36. Towbin, H., Staehelin, T., and Gordon, J. Electrophoretic transfer of proteins from polyacrylamide gels to nitrocellulose sheets: procedure and some applications. PNAS, 76: 4350-4354, 1979.
37. Crawford, C., Brown, N. R., and Willis, A. C. Investigation of the structural basis of the interaction of calpain II with phospholipid and with carbohydrate. Biochem. J., 265: 575-579, 1990.
38. Waseem, A., Lane, E. B., Harrison, D., and Waseem, N. A keratin antibody recognizing a heterotypic complex: Epitope mapping to complementary locations on both components of the complex. Experimental Cell Research, 223: 203-214, 1996.
39. Karlsson, R. A., Michaelsson, A., and Mattsson, L. Kinetic analysis of monoclonal antibody-antigen interactions with a new biosensor based analytical system. J. Immunol. Methods, 145: 229-240, 1991.
40. Marilley, D. and Schwaller, B. Association between the calcium-binding protein calretinin and cytoskeletal components in the human colon adenocarcinoma cell line WiDr. Experimental Cell Research, 259: 12-22, 2000.
41. Chen, P. H., Ornelles, D. A., and Shenk, T. The adenovirus L3 23-kilodalton proteinase cleaves the amino-terminal head domain from cytokeratin 18 and disrupts the cytokeratin network of HeLa cells. J. Virology, 67: 3507-3514, 1993.
42. Zhang, Y. and Schneider, R. J. Adenovirus inhibition of cell translation facilitates release of virus particles and enhances degradation of the cytokeratin network. J. Virology, 68: 2544-2555, 1994.
43. Broers, J. L. V., Ramaekers, F. C. S., Rot, M. K., Oostendorp, T., Huysmans, A., van Muijen, G. N. P., Wagenaar, S. S., and Vooijs, G. P. Cytokeratins in different types of human lung cancer as monitored by chain-specific monoclonal antibodies. Cancer Research, 48: 3221-3229, 1988.
44. Shitara, K., Fujiwara, K., Kusano, A., Yamaguchi, K., Yoshida, H., Sato, S., and Hanai, N. Application of anti lung adenocarcinoma monoclonal antibody recognizing cytokeratin-like cytoplasmic antigen for tumor diagnosis. Anticancer Res, 12: 1121-1129, 1992.
45. Lane, E. B., Goodman, S. L., and Trejdosiewicz, L. K. Disruption of the keratin filament network during epithelial cell division. EMBO Journal, 1: 1365-1372, 1982.
46. Miller, R. K., Khuon, S., and Goldman, R. D. Dynamics of keratin assembly: Exogenous type I keratin rapidly associates with type II keratin in vivo. J. Cell Biology, 122: 123-135, 1993.
47. Nishibori, H., Matsuno, Y., Iwaya, M., Osada, T., Kubomura, N., Iwamatsu, A., Kohno, H., Sato, S., Kitajima, M., and Hirohashi, S. Human colorectal carcinomas specifically accumulate Mr 42,000 ubiquitin-conjugated cytokeratin 8 fragments. Cancer Res., 56: 2752-2757, 1996.
48. Erb, K., Borup-Christensen, P., Ditzel, H., Chemnitz, J., Haas, H., and Jensenius, J. C. Characterization of a human-human hybridoma antibody, C-OU1, directed against a colon tumor-associated antigen. Hybridoma, 11: 121-134, 1992.
49. Ditzel, H. J. Human monoclonal antibodies: a took for cancer detection in vivo. APMIS Suppl., 94: 5-42, 1999.
50. Hembrough, T. A., Kralovich, K. R., Li, L., and Gonias, S. L. Cytokeratin 8 released by breast carcinoma cells in vitro binds plasminogen and tissue-type plasminogen activator and promotes plasminogen activation. Biochem. J., 317: 763-769, 1996.
51. Kralovich, K. R., Li, L., Hembrough, T. A., Webb, D. J., Karns, L. R., and Gonias, S. L. Characterization of the binding sites for plasminogen and tissue-type plasminogen activator in cytokeratin 8 and cytokeratin 18. J. Protein Chem., 17: 845-854, 1998.
52. Wells, M. J., Hatton, M. W. C., Hewlett, B., Podor, T. J., Sheffield, W. P., and Blajchman, M. A. Cytokeratin 18 is expressed on the hepatocyte plasma membrane surface and interacts with thrombin-antithrombin complexes. J. Biological Chemistry, 272: 28574-28581, 1997.
53. Rydlander, L., Ziegler, E., Bergman, T., Schöberl, E., Steiner, G., Bergman, A. C., Zetterberg, A., Marberger, M., Björklund, P., Skem, T., Einarsson, R., and Jörmvall, H. Molecular characterization of a tissue-polypeptide-specific-antigen epitope and its relationship to human cytokeratin 18. Eur. J. Biochem., 241: 309-314, 1996.
54. Silén, Å., Wiklund, B., Norlén, B. J., and Nilsson, S. Evaluation of a new tumor marker for cytokeratin 8 and 18 fragments in healthy individuals and prostate cancer patients. Prostate, 24: 326-332, 1994.
55. Godfroid, E., Geuskens, M., Dupressoir, T., Parent, I., and Szpirer, C. Cytokeratins are exposed on the outer surface of established human mammary carcinoma cells. J. Cell Science, 99: 595-607, 1991.
56. Caulin, C., Salvesen, G. S., and Oshima, G. Caspase cleavage of keratin 18 and reorganization of intermediate filaments during epithelial cell apoptosis. J. Cell Biol., 138: 1379-1394, 1997.
57. Leers, M. P., Kolgen, W., Bjorklund, V., Bergman, T., Tribbick, G., Persson, B., Bjorklund, P., Ramaekers, F. C., Bjorklund, B., Nap, M., Jornvall, H., and Schutte, B. Immunocytochemical detection and mapping of a cytokeratin 18 neo-epitope exposed during early apoptosis. J. Pathol., 187: 567-572, 1999.
58. Ku, N. O. and Omary, M. B. Identification of the major physiologic phosphorylation site of human keratin 18: potential kinases and a role in filament reorganization. J. Cell Biol., 127: 161-171, 1994.
59. Liao, J., Lowthert, L. A., Ku, N. O., Fernandez, R., and Omary, M. B. Dynamics of human keratin 18 phosphorylation: Polarized distribution of phosphorylated keratins in simple epithelial tissues. J. Cell Biology, 131: 1291-1301, 1995.
60. Ku, N. O., Liao, J., Chou, C. F., and Omary, M. B. Implications of intermediate filament protein phosphorylation. Cancer and Metastasis Reviews, 15: 429-444, 1996.

1. An isolated cancer-associated epitope comprising two separate polypeptides, a cytokeratin 8 polypeptide and a cytokeratin 18 polypeptide, wherein the cytokeratin 8 potypeptide consists essentially of SEQ ID NO:3 or SEQ ID NO:5, and the cytokeratin 18 polypeptide consists essentially of SEQ ID NO:4 or SEQ ID NO:6.

2. The isolated epitope of claim 1, wherein the cytokeratin 8 polypeptide is shorter than about 475 amino acids and the cytokeratin 18 polypeptide is shorter than about 425 amino acids.

3. The isolated epitope of claim 1, wherein the cancer-associated epitope is detected in filamentous cytoplasmic structures of adenocarcinoma cells but is substantially undetected in normal cells.

4. The isolated epitope of claim 1, wherein the cancer-associated epitope is detected in filamentous cytoplasmic structures of colon adenocarcinoma, ovarian adenocarcinoma, renal adenocarcinoma, mammary adenocarcinoma, lung adenocarcinoma, pancreatic adenocarcinoma and non-seminomal testis carcinoma cells.

5. A vaccine composition for preventing or treating adenocarcinoma comprising a cancer-associated epitope that comprises two separate polypeptides, a cytokeratin 8 polypeptide and a cytokeratin 18 polypeptide, wherein the cytokeratin 8 polypeptide consists essentially of SEQ ID NO:3 or SEQ ID NO:5, and the cytokeratin 18 polypeptide consists essentially of SEQ ID NO:4 or SEQ ID NO:6.

6. The vaccine composition of claim 5, wherein the cytokeratin 8 polypeptide is shorter than about 475 amino acids and the cytokeratin 18 polypeptide is shorter than about 425 amino acids.

7. The vaccine composition of claim 5, wherein the adenocarcinoma is colon adenocarcinoma, ovarian adenocarcinoma, renal adenocarcinoma, mammary adenocarcinoma, lung adenocarcinoma, pancreatic adenocarcinoma or non-seminomal testis carcinoma.

8. An isolated binding entity polypeptide that can bind to a cancer-associated epitope comprising two separate polypeptides, a cytokeratin 8 polypeptide and a cytokeratin 18 polypeptide, wherein the cytokeratin 8 polypeptide consists essentially of SEQ ID NO:3 or SEQ ID NO:5, and the cytokeratin 18 polypeptide consists essentially of SEQ ID NO:4 or SEQ ID NO:6.

9. The isolated binding entity polypeptide of claim 8, wherein the cytokeratin 8 polypeptide is shorter than about 475 amino acids and the cytokeratin 18 polypeptide is shorter than about 425 amino acids.

10. The isolated binding entity polypeptide of claim 8, wherein the binding entity polypeptide is shorter than about 425 amino acids.

11. The isolated binding entity polypeptide of claim 8, wherein the binding entity polypeptide is shorter than about 200 amino acids.

12. The isolated binding entity polypeptide of claim 8, wherein the binding entity polypeptide is an antibody.

13. The isolated binding entity polypeptide of claim 8, wherein the binding entity polypeptide can detect the cancer-associated epitope in filamentous cytoplasmic structures of adenocarcinoma cells but in substantially no filamentous structures of normal cells.

14. The binding entity of claim 8, wherein the binding entity polypeptide can detect the cancer-associated in filamentous cytoplasmic structures of colon adenocarcinoma, ovarian adenocarcinoma, renal adenocarcinoma, mammary adenocarcinoma, lung adenocarcinoma, pancreatic adenocarcinoma and non-seminomal testis carcinoma cells.

15. The binding entity of claim 8, wherein the binding entity polypeptide consists essentially of a polypeptide having an amino acid sequence with at least 98% homology to any one of SEQ ID NO:7-35.

16. The binding entity of claim 8, wherein the binding entity consists essentially of a polypeptide having any one of SEQ ID NO:7-35 or SEQ ID NO:47-49.

17. The binding entity of claim 8, wherein the binding entity is encoded by a nucleic acid comprising any one of SEQ ID NO:36-39.

18. A kit for detecting cancer comprising a container containing an binding entity polypeptide that can bind to a cancer-associated epitope, wherein the cancer-associated epitope comprises two separate polypeptides, a cytokeratin 8 polypeptide and a cytokeratin 18 polypeptide, and wherein the cytokeratin 8 polypeptide consists essentially of SEQ ID NO:3 or SEQ ID NO:5, and the cytokeratin 18 polypeptide consists essentially of SEQ ID NO:4 or SEQ ID NO:6.

19. The kit of claim 18, wherein the cytokeratin 8 polypeptide is shorter than about 475 amino acids and the cytokeratin 18 polypeptide is shorter than about 425 amino acids.

20. The kit of claim 18, wherein the binding entity polypeptide is shorter than about 425 amino acids.

21. The kit of claim 18, wherein the binding entity potypeptide is shorter than about 200 amino acids.

22. The kit of claim 18, wherein the binding entity polypeptide is an antibody.

23. The kit of claim 18, wherein the cancer is an adenocarcinoma.

24. The kit of claim 18, wherein the cancer is colon adenocarcinoma, ovarian adenocarcinoma, renal adenocarcinoma, mammary adenocarcinoma, lung adenocarcinoma or non-seminomal testis carcinoma.

25. The kit of claim 18, wherein the binding entity polypeptide further comprises a label or diagnostic imaging agent.

26. The kit of claim 18, wherein the binding entity polypeptide further comprises barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, phycoerythrin, phycocyanin, allophycocyanin, ophthaldehyde, fluorescamine, luminal, isoluminal, luciferin, luciferase or aequorin.

27. The kit of claim 18, wherein the binding entity consists essentially of a polypeptide having an amino acid sequence with at least 98% homology to any one of SEQ ID NO:7-35.

28. The kit of claim 18, wherein the binding entity consists essentially of a polypeptide having any one of SEQ ID NO:7-35 or SEQ ID NO:47-49.

29. The kit of claim 18, wherein the binding entity is encoded by a nucleic acid comprising any one of SEQ ID NO:36-39.

30. A therapeutic composition comprising a binding entity polypeptide and a pharmaceutically acceptable carrier, wherein the binding entity polypeptide can bind to a cancer-associated epitope comprising two separate polypeptides, a cytokeratin 8 polypeptide and a cytokeratin 18 polypeptide, wherein the cytokeratin 8 polypeptide comprises SEQ ID NO:3 or SEQ ID NO:5, and the cytokeratin 18 polypeptide comprises SEQ ID NO:4 or SEQ ID NO:6.

31. The therapeutic composition of claim 30, wherein the cytokeratin 8 polypeptide is shorter than about 475 amino acids and the cytokeratin 18 polypeptide is shorter than about 425 amino acids.

32. The therapeutic composition of claims 30, wherein the binding entity polypeptide is shorter than about 425 amino acids.

33. The therapeutic composition of claims 30, wherein the binding entity polypeptide is shorter than about 200 amino acids.

34. The therapeutic composition of claims 30, wherein the binding entity polypeptide is an antibody.

35. The therapeutic composition of claims 30, wherein the binding entity can bind to the cancer-associated epitope in filamentous cytoplasmic strictures of adenocarcinoma cells but in substantially no filamentous structures of normal cells.

36. The therapeutic composition of claims 30, wherein the binding entity can bind to the cancer-associated epitope in filamentous cytoplasmic structures of colon adenocarcinoma, ovarian adenocarcinoma, renal adenocarcinoma, mammary adenocarcinoma, lung adenocarcinoma, pancreatic adenocarcinoma and non-seminomal testis carcinoma cells.

37. The therapeutic composition of claim 30, wherein the binding entity consists essentially of a polypeptide having an amino acid sequence with at least 98% homology to any one of SEQ ID NO:7-35.

38. The therapeutic composition of claim 30, wherein the binding entity consists essentially of a polypeptide having any one of SEQ ID NO:7-35 or SEQ ID NO:47-49.

39. The therapeutic composition of claim 30, wherein the binding entity is encoded by a nucleic acid comprising any one of SEQ ID NO:36-39.

40. A therapeutic composition for treating adenocarcinoma comprising an inhibitor of a protease that cleaves Xaa₁SR↓Xaa₄ (SEQ ID NO:40) and a pharmaceutically acceptable carrier, wherein Xaa₁ is serine, phenylalanine or valine and Xaa₄ is serine or valine.

41. The therapeutic composition of claim 40, wherein the protease is a trypsin-like protease.

42. The therapeutic composition of claim 40, wherein the inhibitor is soybean trypsin inhibitor, alpha-2-macroglobulin, alpha-1-antitrypsin, aprotinin, pancreatic secretory trypsin inhibitor, corn trypsin inhibitor, pumpkin trypsin inhibitor or human amyloid β-protein precursor inhibitor.

43. The therapeutic composition of claim 40, wherein the adenocarcinoma is colon adenocarcinoma, ovarian adenocarcinoma, renal adenocarcinoma, mammary adenocarcinoma, lung adenocarcinoma, pancreatic adenocarcinoma or non-seminomal testis carcinoma.

44. A method of detecting adenocarcinoma comprising contacting a binding entity polypeptide with a test sample and detecting whether the binding entity polypeptide binds to an epitope comprising two separate polypeptides, a cytokeratin 8 polypeptide and a cytokeratin 18 polypeptide, wherein the cytokeratin 8 polypeptide consists essentially of SEQ ID NO:3 or SEQ ID NO:5, and the cytokeratin 18 polypeptide consists essentially of SEQ ID NO:4 or SEQ ID NO:6.

45. The method of claim 44, wherein the cytokeratin 8 polypeptide is shorter than about 475 amino acids and the cytokeratin 18 polypeptide is shorter than about 425 amino acids.

46. The method of claim 44, wherein the binding entity polypeptide is shorter than about 425 amino acids.

47. The method of claim 44, wherein the binding entity polypeptide is shorter than about 200 amino acids.

48. The method of claim 44, wherein the binding entity polypeptide is an antibody.

49. The method of claim 44, wherein the binding entity consists essentially of a polypeptide having an amino acid sequence with at least 98% homology to any one of SEQ ID NO:7-35.

50. The method of claim 44, wherein the binding entity consists essentially of a polypeptide having any one of SEQ ID NO:7-35 or SEQ ID NO:47-49.

51. The method of claim 44, wherein the binding entity is encoded by a nucleic acid comprising any one of SEQ ID NO:36-39.

52. The method of claim 44, wherein the adenocarcinoma is colon adenocarcinoma, ovarian adenocarcinoma, renal adenocarcinoma, mammary adenocarcinoma, lung adenocarcinoma, pancreatic adenocarcinoma or non-seminomal testis carcinoma.

53. The method of claim 44, wherein the binding entity further comprises a label or diagnostic imaging agent.

54. The method of claim 44, wherein the binding entity further comprises barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, phycoerythrin, phycocyanin, allophycocyanin, ophthaldehyde, fluorescamine, luminal, isoluminal, luciferin, luciferase or aequorin.

55. A method of treating or preventing cancer in a mammal comprising administering to the mammal a therapeutically effective amount of a binding entity polypeptide coupled to an anti-neoplastic agent; wherein binding entity polypeptide can bind to a cancer-associated epitope comprising two separate polypeptides, a cytokeratin 8 polypeptide and a cytokeratin 18 polypeptide; and wherein the cytokeratin 8 polypeptide comprises SEQ ID NO:3 or SEQ ID NO:5, and the cytokeratin 18 polypeptide comprises SEQ ID NO:4 or SEQ ID NO:6.

56. The method of claim 55, wherein the cytokeratin 8 polypeptide is shorter than about 475 amino acids and the cytokeratin 18 polypeptide is shorter than about 425 amino acids.

57. The method of claim 55, wherein the binding entity polypeptide is shorter than about 425 amino acids.

58. The method of claim 55, wherein the binding entity polypeptide is shorter than about 200 amino acids.

59. The method of claim 55, wherein the binding entity polypeptide is an antibody.

60. The method of claim 55, wherein the anti-neoplastic agent is radioiodinated compound, a toxin, a cytostatic drug or a cytolytic drug.

61. The method of claim 55, wherein the anti-neoplastic agent is aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon-ax, lomustine, mercaptopurine, methotrexate, mitotane, procarbazine HCl, thioguanine, vinblastine sulfate, vincristine sulfate, pokeweed anti-viral protein, cholera toxin, pertussis toxin, ricin, gelonin, abrin, diphtheria exotoxin, Pseudomonas exotoxin or cobalt-60.

62. The method of claim 55, wherein the binding entity comprises a polypeptide having an amino acid sequence with at least 98% homology to any one of SEQ ID NO:20-35.

63. The method of claim 55, wherein the binding entity comprises a polypeptide having any one of SEQ ID NO:7-35 or SEQ ID NO:47-49.

64. The method of claim 55, wherein the binding entity is encoded by a nucleic acid comprising any one of SEQ ID NO:36-39.

65. A method of identifying a mutant binding entity comprising:
fusing a nucleic acid encoding a polypeptide having any one of SEQ ID NO:7-35 to a nucleic acid encoding a display protein to generate a recombinant nucleic acid encoding a fusion protein;
mutating the recombinant nucleic acid encoding the fusion protein to generate a mutant nucleic acid encoding a mutant fusion protein;
expressing the mutant fusion protein; and
selecting a mutant fusion protein that can bind to a cancer-associated epitope comprising two separate polypeptides, the first polypeptide comprising SEQ ID NO:3 of cytokeratin 8 and the second polypeptide comprising SEQ ID NO:4 of cytokeratin 18.

66. The method of claim 65, wherein the binding entity is a CDR or Fab fragment.

67. The method of claim 65, wherein the display protein is a phage display protein, retroviral display protein, or a ribosomal display protein.

68. Use of the therapeutic composition of claim 30 for preparation of a medicament in the treatment and/or prevention of cancer in a mammal.

69. Use of the therapeutic composition of claim 40 for preparation of a medicament in the treatment and/or prevention of cancer in a mammal.


SEQUENCE LISTING (64 sequences)

SEQ ID NO: 1 - protein, 482 aa, Homo sapiens
SIRVTQKSYKVSTSGPRAFSSRSYTSGPGSRISSSSFSRVGSSNFRGGLGGGYGGASGMGGITAVTVNQSLLSPLSLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMESIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIDIATYRKLLEGEESPLESGMQNMSIHTKTTGGYAGGLSSAYGDLTDPGLSYSLGSSFGSGAGSSSFSRTSSSRAVVVKKIETRDGKLVSESSDVLPK

SEQ ID NO: 2 - protein, 429 aa, Homo sapiens
SFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 3 - protein, 44 aa, Homo sapiens
AVRTQEKEQIKTLNNKFASFIDKVRFLEQQNKMLETKWSLLQQQ

SEQ ID NO: 4 - protein, 53 aa, Homo sapiens
AGMGGIQNEKETMQSLNDRLASYLDRVRSLETENRRLESKIREHLEKKGPQVR

SEQ ID NO: 5 - protein, 398 aa, Homo sapiens
AVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMESIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIDIATYRKLLEGEESPLESGMQNMSIHTKTTGGYAGGLSSAYGDLTDPGLSYSLGSSFGSGAGSSSFSRTSSSRAVVVKKIETRDGKLVSESSDVLPK

SEQ ID NO: 6 - protein, 359 aa, Homo sapiens
AGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 7 - protein, 23 aa, Homo sapiens
GAEVKKPGASVKVSCKASDYTFS

SEQ ID NO: 8 - protein, 5 aa, Homo sapiens
SYYMH

SEQ ID NO: 9 - protein, 14 aa, Homo sapiens
WVRQAPGQGLEWMG

SEQ ID NO: 10 - protein, 17 aa, Homo sapiens
IINPSGGSTSYAQKFQG

SEQ ID NO: 11 - protein, 32 aa, Homo sapiens
RVTMTRDTSTNTVYMELSSLRSEDTAVYYCAR

SEQ ID NO: 12 - protein, 16 aa, Homo sapiens
DQVVVAATLSNYGMDV

SEQ ID NO: 13 - protein, 14 aa, Homo sapiens
WGQGTTVTVSSAST

SEQ ID NO: 14 - protein, 21 aa, Homo sapiens
ELTQSPGTLSLSPGERATLSC

SEQ ID NO: 15 - protein, 12 aa, Homo sapiens
RASQSVSSSYLA

SEQ ID NO: 16 - protein, 15 aa, Homo sapiens
WYQQKPGQAPRLLIY

SEQ ID NO: 17 - protein, 7 aa, Homo sapiens
DASNRAT

SEQ ID NO: 18 - protein, 32 aa, Homo sapiens
GIPARFSGSGSGTDFTLTISSLEPEDFAVYYC

SEQ ID NO: 19 - protein, 9 aa, Homo sapiens
QQGTNWGIA

SEQ ID NO: 20 - protein, 11 aa, Homo sapiens
FGQGTRLDIKR

SEQ ID NO: 21 - protein, 19 aa, Homo sapiens
TQSPGTLSLSPGERATLSC

SEQ ID NO: 22 - protein, 7 aa, Homo sapiens
GASSRAT

SEQ ID NO: 23 - protein, 32 aa, Homo sapiens
GIPDRFSGSGSGTDFTLTISRLEPEDFAAYYC

SEQ ID NO: 24 - protein, 10 aa, Homo sapiens
QQYGNSPPYT

SEQ ID NO: 25 - protein, 9 aa, Homo sapiens
FGQGTKLEI

SEQ ID NO: 26 - protein, 19 aa, Homo sapiens
TQSPDSLAVSLGERATINC

SEQ ID NO: 27 - protein, 17 aa, Homo sapiens
KSSQSLLYSSNNKNYLA

SEQ ID NO: 28 - protein, 15 aa, Homo sapiens
WYQQKPGQPPKLLIY

SEQ ID NO: 29 - protein, 7 aa, Homo sapiens
WASTRES

SEQ ID NO: 30 - protein, 13 aa, Homo sapiens
GVPDRFSGSGSGT

SEQ ID NO: 31 - protein, 19 aa, Homo sapiens
DFTLTISSLQAEDVAGYYC

SEQ ID NO: 32 - protein, 9 aa, Homo sapiens
QQYYSTPPM

SEQ ID NO: 33 - protein, 9 aa, Homo sapiens
FGQGTKVEI

SEQ ID NO: 34 - protein, 104 aa, Homo sapiens
TQSPGTLSLSPGERATLSCRASQSVSSSYLAWYQQKPGQAPRLLIYGASSRATGIPDRFSGSGSGTDFTLTISRLEPEDFAAYYCQQYGNSPPYTFGQGTKLEI

SEQ ID NO: 35 - protein, 108 aa, Homo sapiens
TQSPDSLAVSLGERATINCKSSQSLLYSSNNKNYLAWYQQKPGQPPKLLIYWASTRESGVPDRFSGSGSGTDFTLTISSLQAEDVAGYYCQQYYSTPPMFGQGTKVEI

SEQ ID NO: 36 - DNA, 354 bp, Homo sapiens
ggggctgaggtgaagaagcctggggcgtcagtgaaggtttcctgcaaggcatctggatacaccttcagcagctactatatgcactgggtgcgacaggcccctggacaagggcttgagtggatgggaataatcaaccctagtggtggtagcacaagctacgcacagaagttccagggcagagtcaccatgaccagggacacgtccacgaacacagtctacatggagctgagcagcctgagatctgaggacacggccgtgtattactgtgcgagagatcaggtggtggtagctgctactttgtccaactacggtatggacgtctggggccaagggaccacggtcaccgtctcctca

SEQ ID NO: 37 - DNA, 321 bp, Homo sapiens
gagctcacccagtctccaggcaccctgtctttgtctccaggggaaagagccaccctctcctgcagggccagtcagagtgttagtagcagctacttagcctggtaccagcagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagggtaccaactgggggatcgccttcggccaagggacacgactggatattaaacga

SEQ ID NO: 38 - DNA, 313 bp, Homo sapiens
acgcagtctccaggcaccctgtctttgtctccaggggaaagagccaccctctcctgtagggccagtcagagtgttagcagcagctacttagcctggtaccagcagaaacctggccaggctcccaggctcctcatctatggtgcatccagcagggccactggcatcccagacaggttcagtggcagtgggtcagggacagacttcactctcaccatcagcagactggagcctgaagattttgcagcgtattactgtcagcagtatggtaactcacctccgtacacttttggccaggggaccaagctggagatca

SEQ ID NO: 39 - DNA, 324 bp, Homo sapiens
acccagtctccagactccctggctgtgtctctgggcgagagggccaccatcaactgcaagtccagccagagtcttttatacagctccaacaataagaactacttagcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagatgtggcaggttattactgtcagcaatattatagtactcctccgatgttcggccaagggaccaaggtggaaatc

SEQ ID NO: 40 - protein, 4 aa, Artificial Sequence
  A synthetic consensus sequence
XSRX

SEQ ID NO: 41 - DNA, 21 bp, Artificial Sequence
  A synthetic primer
atagaagttgttcagcaggca

SEQ ID NO: 42 - DNA, 22 bp, Artificial Sequence
  A synthetic primer
gaattctaaactagctagttcg

SEQ ID NO: 43 - DNA, 21 bp, Artificial Sequence
  A synthetic primer
caagggcttgagtggatggga

SEQ ID NO: 44 - DNA, 17 bp, Artificial Sequence
  A synthetic primer
attaaccctcactaaag

SEQ ID NO: 45 - DNA, 1724 bp, Homo sapiens
ttcggcaattcctacctccactcctgcctccaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagccccttgtccctggaggtggaccccaacatccaggccgtgcgcacccaggagaaggagcagatcaagaccctgaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcatacatgaacaaggtagagctggagtctcgcctggaagggctgaccgacgagatcaacttcctcaggcagctgtatgaagaggagatccgggagctgcagtcccagatctcggacacatctgtggtgctgtccatggacaacagccgctccctggacatggagagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctcagagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcccggcagctgcgtgagtaccaggagctgatgaacgtcaagctggccctggacatcgacatcgccacctacaggaagctgctggagggcgaggagagcccgctggagtctgggatgcagaacatgagtattcatacgaagaccaccggcggctatgcgggtggtttgagctcggcctatggggacctcacagaccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttttggtcccaaaataaaacctcagctagctctgcc

SEQ ID NO: 46 - DNA, 1412 bp, Homo sapiens
cggggtcgtccgcaaagcctgagtcctgtcctttctctctccccggacagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaagcagggtaccctttggggagcaggaggccaataaaaagttcagagttcattggatgtc

SEQ ID NO: 47 - protein, 17 aa, Artificial Sequence
  A synthetic formula for related variable light chain CDR1 fragments and binding entities
XXXXXXXXXXXXXXXXX

SEQ ID NO: 48 - protein, 7 aa, Artificial Sequence
  A synthetic formula for related variable light chain CDR2 fragments and binding entities
XXXXXXX

SEQ ID NO: 49 - protein, 10 aa, Artificial Sequence
  A synthetic formula for related variable light chain CDR3 fragments and binding entities
XXXXXXXXXX

SEQ ID NO: 50 - protein, 6 aa, Artificial Sequence
  A synthetic consensus sequence
XXSRXX

SEQ ID NO: 51 - protein, 4 aa, Homo sapiens
TAVT

SEQ ID NO: 52 - protein, 4 aa, Homo sapiens
SPLV

SEQ ID NO: 53 - protein, 4 aa, Homo sapiens
TGIA

SEQ ID NO: 54 - protein, 10 aa, Homo sapiens
SYTSGPGSRI

SEQ ID NO: 55 - protein, 10 aa, Homo sapiens
VGSSNFRGGL

SEQ ID NO: 56 - protein, 10 aa, Homo sapiens
TVNQSLLSPL

SEQ ID NO: 57 - protein, 10 aa, Homo sapiens
VLEVDPNIQA

SEQ ID NO: 58 - protein, 15 aa, Homo sapiens
STSFRGGMGSGGLAT

SEQ ID NO: 59 - protein, 20 aa, Homo sapiens
AGGLAGMGGIQNEKETMQSL

SEQ ID NO: 60 - protein, 10 aa, Homo sapiens
FGPGVAFRAP

SEQ ID NO: 61 - protein, 8 aa, Homo sapiens
MLTELEKA

SEQ ID NO: 62 - protein, 105 aa, Homo sapiens
SIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFI

SEQ ID NO: 63 - protein, 94 aa, Homo sapiens
SFTTRSTFSTNYRSLGSVQAPSYGARPVSSAASVYAGAGGSGSRISVSRSTSFRGGMGSGGLATGIAGGLAGMGGIQNEKETMQSLNDRLASYL

SEQ ID NO: 64 - protein, 4 aa, Homo sapiens
GSRI